(19) United States
(12) Patent Application Publication
(10) Pub. No.: US 2006/0199853 A1
(43) Pub. Date: Sep. 7, 2006

(54) Analogs of 4-hydroxyisoleucine and uses thereof

(76) Inventors: Charles Mioskowski, Strasbourg (FR); Sandra De Lamo Marin, La Jolla, CA (US); Martine Maruani, Calais (FR); Manjinder Gill, Laval (CA)

Correspondence Address: CLARK & ELBING LLP, 101 FEDERAL STREET, BOSTON, MA, 02110, US

(21) Appl. No.: 11/356,848

(22) Filed: Feb. 17, 2006

Related U.S. Application Data
(60) Provisional application No. 60/654,342, filed on Feb. 18, 2005.

(51) Int. Cl. A61K 31/41; C07D 233/61; A61K 31/4172; A61K 31/198

(52) U.S. Cl. 514/381; 514/561; 514/469; 562/507; 514/383; 514/400; 548/253; 548/335.5

(57) ABSTRACT

The invention relates to analogs of 4-hydroxyisoleucine, and to lactones, pharmaceutically acceptable salts, and prodrugs thereof, to processes for their preparation, and to pharmaceutical compositions comprising the same. The analogs of the invention stimulate both glucose uptake and insulin secretion, and may thus be useful for the prevention and treatment of disorders of carbohydrate or lipid metabolism, including diabetes mellitus (type 1 and type 2 diabetes), pre-diabetes, and Metabolic Syndrome.

### RELATED APPLICATION

This application claims priority from U.S. Provisional Application 60/654,342 filed Feb. 18, 2005, the disclosure of which is incorporated by reference herein in its entirety.

### BACKGROUND OF THE INVENTION

a) Field of the Invention

The invention relates to analogs of 4-hydroxyisoleucine, and to lactones, pharmaceutically acceptable salts and prodrugs thereof, to processes for their preparation, to pharmaceutical compositions comprising the same and to their use for preventing and treating disorders of carbohydrate or lipid metabolism, including diabetes mellitus (type 1 and type 2 diabetes), pre-diabetes, and Metabolic Syndrome.

b) Brief Description of the Related Art

Diabetes mellitus is a disorder of carbohydrate metabolism, and develops when the body cannot effectively control blood glucose levels. The disease is characterized by inadequate secretion or utilization of insulin, high glucose levels in the blood and urine, and excessive thirst, hunger, weight loss, and urine production. It can lead to a number of serious complications, including cardiovascular disease, kidney disease, blindness, nerve damage, and limb ischemia. Diabetes is divided into two types, 1 and 2, with the latter accounting for about 90% of the cases. In type 1 diabetes, the body destroys the insulin-producing β-cells of the pancreas, resulting in the inability of the body to produce insulin. Type 1 diabetes typically occurs in children or young adults, and generally is managed by insulin administration, strict diet, and exercise. Type 1 diabetes is observed as well in older adults following therapeutic failure of type 2 diabetes. Type 2 diabetes is characterized by impaired insulin secretion due to altered β-cell function, as well as decreased ability of normally insulin sensitive tissues (e.g., the liver and muscle) to respond to insulin. Type 2 diabetes generally develops in those over 45, but is recently also being detected in younger people. The disease is associated with risk factors such as age, family history, obesity, lack of regular exercise, high blood pressure, and hyperlipidemia. Treatment involves strict diet and exercise regimens, oral medications (e.g., medications that increase insulin secretion and/or insulin sensitivity), and, in some cases, insulin administration.

Type 2 diabetes is rapidly increasing in its importance as a major public health concern in the Western world. While one hundred years ago it was a relatively rare disease, today there are more than 200 million type 2 diabetics worldwide, and this number is estimated to increase to greater than about 300 million by the year 2025. This dramatic increase in the incidence of type 2 diabetes parallels an increase in the prevalence of obesity in Western cultures. Further, as more cultures adopt Western dietary habits, it is likely that type 2 diabetes will reach epidemic proportions throughout the world. Given the seriousness of the complications associated with this disease, as well as its rapidly increasing incidence, the development of effective approaches to treatment is a primary concern in the field of medicine.

In 1973, Fowden et al., in Phytochemistry 12:1707-1711, 1973, reported the presence of (2S,3R,4R)-4-hydroxy-3-methylpentanoic acid (4-hydroxyisoleucine) in the seeds of fenugreek (Trigonella foenumgraecum), an annual herbaceous plant that is widespread in regions of Asia, Africa, and Europe. Its absolute configuration was subsequently restudied and corrected as being (2S,3R,4S) by Alcock et al. in Phytochemistry 28:1835-1841, 1989. It has been demonstrated that (2S,3R,4S)-4-hydroxyisoleucine possesses insulinotropic and insulin sensitizing activities (see Broca et al., Am. J. Physiol. 277:E617-E623, 1999; Broca et al., Eur. J. Pharmacol. 390:339-345, 2000; Broca et al., Am. J Physiol. Endocrinol. Metab. 287:E463-E471, 2004) and that compound has since been developed for the treatment of diabetes (U.S. Pat. No. 5,470,879; PCT publication Nos. WO 97/32577, WO 01/15689, and WO-2005/039626). Although methods for the preparation of (2S,3R,4S)-4-hydroxyisoleucine have been described, see for example U.S. Patent Application Publication No. US 2003/0219880; Rolland-Fulcrand et al., Eur. J. Org. Chem. 873-877, 2004; and Wang et al., Eur. J. Org. Chem. 834-839, 2002, no one has ever disclosed synthetic analogs of 4-Hydroxyisoleucine, let alone analogs useful for the prevention and/or treatment of metabolic diseases such as diabetes.

In view of the above, there is an important need for alternative and improved compounds for preventing and treating disorders of carbohydrate or lipid metabolism, particularly diabetes.

There is also a need for pharmaceutical compositions and therapeutic methods of stimulating glucose uptake and/or of stimulating insulin secretion.

The present invention provides such compounds along with methods for their use. Accordingly, the present invention fulfils the above-mentioned needs and also other needs as it will be apparent to those skilled in the art upon reading the following specification.

### SUMMARY OF THE INVENTION

The invention provides analogs of (2S,3R,4S)-4-hydroxyisoleucine (4-OH) and their use in compositions and methods for treating disorders of carbohydrate or lipid metabolism, including diabetes mellitus (type 1 and type 2 diabetes), pre-diabetes, and Metabolic Syndrome.

Accordingly, a first aspect of the present invention features analogs of 4-hydroxyisoleucine, such as those having Formula (I): 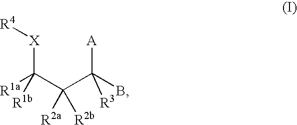
and pharmaceutically acceptable lactones, salts, metabolites or prodrugs thereof, wherein in said Formula (I):

A is CO₂R^{A1}, C(O)SR^{A1}, C(S)SR^{A1}, C(O)NR^{A2}R^{A3}, C(S)NR^{A2}R^{A3}, C(O)R^{A4}, SO₃H, S(O)₂NR^{A2}R^{A3}, C(O)R^{A5}, C(OR^{A1})R^{A9}R^{A10}, C(SR^{A1})R^{A9}R^{A10}, C(═NR^{A1})R^{A5},

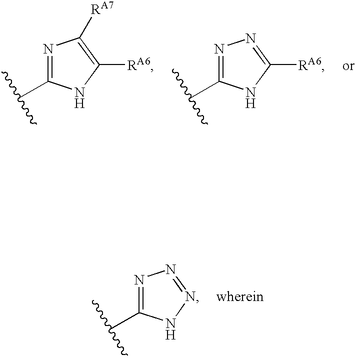

R^{A1} is hydrogen, substituted or unsubstituted C₁₋₆ alkyl, substituted or unsubstituted C₃₋₈ cycloalkyl, substituted or unsubstituted alkcycloalkyl, where the cycloalkyl group is of three to eight carbon atoms and the alkylene group is of one to four carbon atoms, substituted or unsubstituted C₂₋₆ alkenyl, substituted or unsubstituted C₂₋₆ alkynyl, substituted or unsubstituted C₆ or C₁₀ aryl, substituted or unsubstituted C₇₋₁₆ alkaryl, where the alkylene group is of one to four carbon atoms, substituted or unsubstituted C₁₋₉ heterocyclyl, or substituted or unsubstituted C₂₋₁₅ alkheterocyclyl, where the alkylene group is of one to four carbon atoms,

each of R^{A2} and R^{A3} is, independently, selected from the group consisting of (a) hydrogen, (b) substituted or unsubstituted C₁₋₆ alkyl, (c) substituted or unsubstituted C₃₋₈ cycloalkyl, (d) substituted or unsubstituted alkcycloalkyl, where the cycloalkyl group is of three to eight carbon atoms and the alkylene group is of one to four carbon atoms, (e) substituted or unsubstituted C₆ or C₁₀ aryl, and (f) substituted or unsubstituted C₇₋₁₆ alkaryl, where the alkylene group is of one to six carbon atoms, or R^{A2} taken together with R^{A3} and N forms a substituted or unsubsituted 5- or 6-membered ring, optionally containing O or NR^{A8}, wherein R^{A8} is hydrogen or C₁₋₄ alkyl,

R^{A4} is substituted or unsubstituted C₁₋₆ alkyl, substituted or unsubstituted C₃₋₈ cycloalkyl, substituted or unsubstituted alkcycloalkyl, where the cycloalkyl group is of three to eight carbon atoms and the alkylene group is of one to four carbon atoms, substituted or unsubstituted C₆ or C₁₀ aryl, substituted or unsubstituted C₇₋₁₆ alkaryl, where the alkylene group is of one to four carbon atoms, substituted or unsubstituted C₁₋₉ heterocyclyl, or substituted or unsubstituted C₂₋₁₅ alkheterocyclyl, where the alkylene group is of one to four carbon atoms,

R^{A5} is a peptide chain of 1-4 natural or unnatural amino acids, where the peptide is linked via its terminal amine group to C(O),

each of R^{A6} and R^{A7} is, independently, hydrogen, substituted or unsubstituted C₁₋₆ alkyl, C₁₋₄ perfluoroalkyl, substituted or unsubstituted C₁₋₆ alkoxy, amino, C₁₋₆ alkylamino, C₂₋₁₂ dialkylamino, N-protected amino, halo, or nitro, and

each of R^{A9} and R^{A10} is, independently, selected from the group consisting of (a) hydrogen, (b) substituted or unsubstituted C₁₋₆ alkyl, (c) substituted or unsubstituted C₃₋₈ cycloalkyl, (d) substituted or unsubstituted alkcycloalkyl, where the cycloalkyl group is of three to eight carbon atoms and the alkylene group is of one to four carbon atoms, (e) substituted or unsubstituted C₆ or C₁₂ aryl, and (D substituted or unsubstituted C₇₋₁₆ alkaryl, where the alkylene group is of one to six carbon atoms, or R^{A9} taken together with R¹⁰ and their parent carbon atom forms a substituted or unsubsituted 5- or 6-membered ring, optionally containing 0 or NR^{A8}, wherein R^{A8} is hydrogen or C₁ alkyl;

B is NR^{B1}R^{B2}, where

(i) each of R^{B1} and R^{B2} is, independently selected from the group consisting of (a) hydrogen, (b) an N-protecting group, (c) substituted or unsubstituted C₁₋₆ alkyl, (d) substituted or unsubstituted C₂₋₆ alkenyl, (e) substituted or unsubstituted C₂₋₆ alkynyl, (f) substituted or unsubstituted C₃₋₈ cycloalkyl, (g) substituted or unsubstituted alkcycloalkyl, where the cycloalkyl group is of three to eight carbon atoms, and the alkylene group is of one to ten carbon atoms, (h) substituted or unsubstituted C₆ or C₁₀ aryl, (i) substituted or unsubstituted C₇₋₁₆ alkaryl, where the alkylene group is of one to six carbon atoms, (j) substituted or unsubstituted C₁₋₉ heterocyclyl, (k) substituted or unsubstituted C₂₋₁₅ alkheterocyclyl, where the alkylene group is of one to six carbon atoms, (I) C(O)R^{B3}, where R^{B3} is selected from the group consisting of substituted or unsubstituted C₁₋₆ alkyl, substituted or unsubstituted C₆ or C₁₀ aryl, substituted or unsubstituted C₇₋₁₆ alkaryl, where the alkylene group is of one to six carbon atoms, substituted or unsubstituted C₁₋₉ heterocyclyl, or substituted or unsubstituted C₂₋₁₅ alkheterocyclyl, where the alkylene group is of one to six carbon atoms, (m) CO₂R^{B4}, where R^{B4} is selected from the group consisting of substituted or unsubstituted C₁₋₆ alkyl, substituted or unsubstituted C₆ or C₁₀ aryl, substituted or unsubstituted C₇₋₁₆ alkaryl, where the alkylene group is of one to six carbon atoms, substituted or unsubstituted C₁₋₉ heterocyclyl, or substituted or unsubstituted C₂₋₁₅ alkheterocyclyl, where the alkylene group is of one to six carbon atoms, (n) C(O)NR^{B5}R^{B6}, where each of R^{B5} and R^{B6} is, independently, selected from the group consisting of hydrogen, substituted or unsubstituted C₁₋₆ alkyl, substituted or unsubstituted C₆ or C₁₀ aryl, substituted or unsubstituted C₇₋₁₆ alkaryl, where the alkylene group is of one to six carbon atoms, substituted or unsubstituted C₁₋₉ heterocyclyl, and substituted or unsubstituted C₂₋₁₅ alkheterocyclyl, where the alkylene group is of one to six carbon atoms, or R^{B5} taken together with R^{B6} and N forms a substituted or unsubsituted 5- or 6-membered ring, optionally containing a non-vicinal O, S, or NR′, where R′ is H or C₁₋₆ alkyl, (o) S(O)₂R^{B7}, where R^{B7} is selected from the group consisting of substituted or unsubstituted C₁₋₆ alkyl, substituted or unsubstituted C₆ or C₁₀ aryl, substituted or unsubstituted C₇₋₁₆ alkaryl, where the alkylene group is of one to six carbon atoms, substituted or unsubstituted C₁₋₉ heterocyclyl, or substituted or unsubstituted C₂₋₁₅ alkheterocyclyl, where the alkylene group is of one to six carbon atoms, and (p) a peptide chain of 1-4 natural or unnatural alpha-amino acid residues, where the peptide is linked via its terminal carboxy group to N, with the proviso that no two groups are bound to the nitrogen atom through a carbonyl group or a sulfonyl group, or

(ii) R^{B1} taken together with R^{B2} and N forms a substituted or unsubstituted 5- or 6-membered ring, optionally containing O or NR^{B8}, wherein R^{B8} is hydrogen or C₁₋₆ alkyl, or

(iii) a 5- to 8-membered ring is formed when R^{B1} taken together with R^{1a} is a substituted or unsubstituted C₁₋₄ alkylene, or

(iv) a [2.2.1] or [2.2.2] bicyclic ring system is formed when R^{B1} taken together with R^{1a} is a substituted or unsubstituted C₂ alkylene and R^{B1} taken together with R^{2a} is a substituted or unsubstituted C₁₋₂ alkylene, or

(v) a 4- to 8-membered ring is formed when R^{B1} taken together with R³ is a substituted or unsubstituted C₂₋₆ alkylene, or

(vi) a 6- to 8-membered ring is formed when R^{B1} taken together with R⁴ is a substituted or unsubstituted C₁₋₃ alkylene, or

(vii) R^{B1} taken together with A and the parent carbon of A and B forms the following ring: 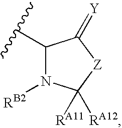
wherein each of Y and Z is, independently, O, S, NR^{B8}, or CR^{A9}R^{A10}, wherein each of R^{A9} and R^{A10} is as previously defined and each of R^{A11} and R^{A12} is, independently, selected from the group consisting of (a) hydrogen, (b) substituted or unsubstituted C₁₋₆ alkyl, (c) substituted or unsubstituted C₃₋₈ cycloalkyl, (d) substituted or unsubstituted alkcycloalkyl, where the cycloalkyl group is of three to eight carbon atoms and the alkylene group is of one to four carbon atoms, (e) substituted or unsubstituted C₆ or C₁₀ aryl, and (f) substituted or unsubstituted C₇₋₁₆ alkaryl, where the alkylene group is of one to six carbon atoms, or R^{A9} taken together with R^{A10} and their parent carbon atom forms a substituted or unsubsituted 5- or 6-membered ring, optionally containing O or NR^{A8}, where R^{A8} is hydrogen or C₁₋₆alkyl;

X is O, S, or NR^{X1}, where R^{X1} is selected from the group consisting of (a) hydrogen, (b) an N-protecting group, (c) substituted or unsubstituted C₁₋₆ alkyl, (d) substituted or unsubstituted C₂₋₆ alkenyl, (e) substituted or unsubstituted C₂₋₆ alkynyl, (f) substituted or unsubstituted C₃₋₈ cycloalkyl, (g) substituted or unsubstituted alkcycloalkyl, where the cycloalkyl group is of three to eight carbon atoms, and the alkylene group is of one to ten carbon atoms, (h) substituted or unsubstituted C₆ or C₁₀ aryl, (i) substituted or unsubstituted C₇₋₁₆ alkaryl, where the alkylene group is of one to six carbon atoms, (j) substituted or unsubstituted C₁₋₉ heterocyclyl,or (k) substituted or unsubstituted C₂₋₁₅ alkheterocyclyl, where the alkylene group is of one to six carbon atoms;

each of R^{1a} and R^{1b} is, independently, substituted or unsubstituted C₁₋₆ alkyl, substituted or unsubstituted C₃₋₈ cycloalkyl, substituted or unsubstituted alkcycloalkyl, where the cycloalkyl group is of three to eight carbon atoms and the alkylene group is of one to four carbon atoms, substituted or unsubstituted C₂₋₆alkenyl, substituted or unsubstituted C₂₋₆ alkynyl, substituted or unsubstituted C₆ or C₁₀ aryl, substituted or unsubstituted C₇₋₁₆ alkaryl, where the alkylene group is of one to four carbon atoms, substituted or unsubstituted C₁₋₉ heterocyclyl, or substituted or unsubstituted C₂₋₁₅ alkheterocyclyl, where the alkylene group is of one to four carbon atoms, or R^{1a} together with R^{2a} and their base carbon atoms form a substituted or unsubstituted C₅₋₁₀ mono or fused ring system, or a 3- to 6-membered ring is formed when R^{1a} together with R⁴ is a substituted or unsubstituted C₁₋₄ alkylene;

each of R^{2a} and R^{2b} is, independently, hydrogen, substituted or unsubstituted C₁₋₆ alkyl, substituted or unsubstituted C₃₋₈ cycloalkyl, substituted or unsubstituted alkcycloalkyl, where the cycloalkyl group is of three to eight carbon atoms and the alkylene group is of one to four carbon atoms, substituted or unsubstituted C₂₋₆ alkenyl, substituted or unsubstituted C₂₋₆ alkynyl, substituted or unsubstituted C₆ or C₁₀ aryl, substituted or unsubstituted C₇₋₁₆ alkaryl, where the alkylene group is of one to four carbon atoms, substituted or unsubstituted C₁₋₉ heterocyclyl, or substituted or unsubstituted C₂₋₁₅ alkheterocyclyl, where the alkylene group is of one to four carbon atoms, or R^{2a} and R^{2b} together are ═O, ═N(C₁₋₆ alkyl), ═CR^{2c}R^{2d}, where each of R^{2c} and R^{2d} is, independently, hydrogen or substituted or unsubstituted C₁₋₆ alkyl, or a substituted or unsubstitued C₂₋₅ alkylene moiety forming a spiro ring, or R^{2a} together with R^{1a} and their base carbon atoms form a substituted or unsubstituted C₅₋₁₀ mono or fused ring system;

R³ is hydrogen, substituted or unsubstituted C₁₋₆ alkyl, substituted or unsubstituted alkcycloalkyl, where the cycloalkyl group is of three to eight carbon atoms and the alkylene group is of one to four carbon atoms, substituted or unsubstituted C₂₋₆ alkenyl, substituted or unsubstituted C₂₋₆ alkynyl, substituted or unsubstituted C₇₋₁₆ alkaryl, where the alkylene group is of one to four carbon atoms, or substituted or unsubstituted C₂₋₁₅ alkheterocyclyl, where the alkylene group is of one to four carbon atoms; and

R⁴ is hydrogen, substituted or unsubstituted C₁₋₆ alkyl, substituted or unsubstituted C₃₋₈ cycloalkyl, substituted or unsubstituted alkcycloalkyl, where the cycloalkyl group is of three to eight carbon atoms and the alkylene group is of one to four carbon atoms, substituted or unsubstituted C₂₋₆ alkenyl, substituted or unsubstituted C₂₋₆ alkynyl, substituted or unsubstituted C₆ or C₁₀ aryl, substituted or unsubstituted C₇₋₁₆ alkaryl, where the alkylene group is of one to four carbon atoms, substituted or unsubstituted C₁₋₉ heterocyclyl, or substituted or unsubstituted C₂₋₁₅ alkheterocyclyl, where the alkylene group is of one to four carbon atoms, or a 3- to 6-membered ring is formed when R⁴ together with R^{1a} is a substituted or unsubstituted C₁₋₄ alkylene, or a 6- to 8-membered ring is formed when R⁴ taken together with R^{B1} is a substituted or unsubstituted C₁₋₃ alkylene,

with the proviso that said compound of Formula (I) is not an isomer of 4-hydroxyisoleucine nor 4-hydroxyisoleucine γ-lactone.

In one embodiment, the R^{B1} substituent does not form rings with R^{1a}, or R⁴.

In an other embodiment, the compound of Formula (I) is a prodrug, preferably a 5-membered ring lactone or a thiolactone, such as those which are formed when A and X—R⁴ together form a C(O)O or C(O)S linkage, respectively.

In an other embodiment, the analog of 4-OH is a compound of Formula (II): 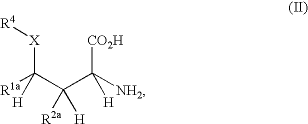
or a pharmaceutically acceptable lactone, salt, metabolite or prodrug thereof, wherein in said Formula (II), each of R^{1a} and R^{2a} is, independently, substituted or unsubstituted C₁₋₆ alkyl or R^{1a} together with R^{2a} and their base carbon atoms form a substituted or unsubstituted-6-membered ring.

Yet, in another embodiment, the analog of 4-OH is a compound of Formula (III) 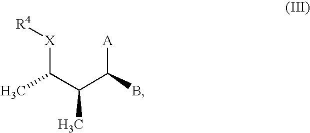
or a pharmaceutically acceptable lactone, salt, metabolite or prodrug thereof, wherein in each of A, B, X, and R⁴ are as defined previously.

In another embodiment, the analog of 4-OH is a compound of Formula (IV): 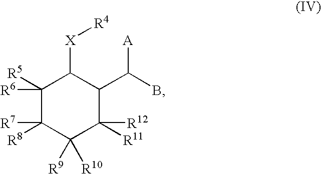
where each of B, X, and R⁴ is as defined elsewhere herein, A is CO₂R^{A1}, C(O)SR^{A1}, C(O)NR^{A2}R^{A3}, or C(O)R^{A5}, and R⁵, R⁶, R⁷, R⁸, R⁹, R¹⁰, R¹¹, and R¹² are, independently, hydrogen, substituted or unsubstituted C₁₋₆ alkyl, substituted or unsubstituted C₃₋₈ cycloalkyl, substituted or unsubstituted alkcycloalkyl, where the cycloalkyl group is of three to eight carbon atoms and the alkylene group is of one to four carbon atoms, substituted or unsubstituted C₂₋₆ alkenyl, substituted or unsubstituted C₂₋₆ alkynyl, substituted or unsubstituted C₆ or C₁₀ aryl, substituted or unsubstituted C₇₋₁₆ alkaryl, where the alkylene group is of one to four carbon atoms, substituted or unsubstituted C₁₋₉ heterocyclyl, or substituted or unsubstituted C₂₋₁₅ alkheterocyclyl, where the alkylene group is of one to four carbon atoms.

In other embodiments, the analogs of the invention are selected from the specific compounds listed hereinafter in Table 1.

According, to a second aspect of the present invention features the use of analogs of 4-hydroxyisoleucine as defined herein, for therapeutic and/or prophylactic purposes. In one embodiment, there is provided a method for treating a mammal having a disorder of carbohydrate or lipid metabolism that includes administering to the mammal one or more analog of 4-OH as defined herein. Preferably, the disorder is non-insulin dependent diabetes mellitus, more preferably type 2 diabetes mellitus. According to another aspect, the invention is directed to a method of treatment of disease in a mammal treatable by administration a compound stimulating insulin secretion, which method comprises administration of a therapeutically effective amount of a pharmaceutical composition comprising a therapeutically effective amount of at least one analog of 4-OH according to the invention, and a pharmaceutically acceptable carrier or excipient, either alone or in combination with other pharmacologically active agents

In another aspect, this invention is directed to a method for stimulating glucose uptake by muscle cells and/or adipocytes, comprising contacting such cells with an effective amount of analog(s) according to the invention.

In another aspect, this invention is directed to a method for stimulating insulin secretion by beta-cells in the pancreatic islets, comprising contacting said cells with an effective amount of analog(s) according to the invention.

In yet another aspect, this invention is directed to pharmaceutical compositions and more particularly to the use of analog(s) according to the invention in the preparation of a medicine for use in the treatment of a disorder of carbohydrate or lipid metabolism in which elevated circulating glucose levels are problematic, including but not limited to diabetes mellitus (type 1 and type 2 diabetes), pre-diabetes, Metabolic Syndrome, hyperglycemia, diabetic neuropathy and diabetic nephropathy.

In a further aspect of the present invention there are provided processes for the preparation of analog(s) according to the invention.

An advantage of the invention is that it provides novel useful stimulators of glucose uptake and stimulators of insulin secretion. The invention also provides compounds, compositions and methods for the unmet medical need of carbohydrate or lipid metabolism, and more particularly type 2 diabetes.

Additional objects, advantages and features of the present invention will become more apparent upon reading of the following non-restrictive description of preferred embodiments with reference to the accompanying drawings which are exemplary and should not be interpreted as limiting the scope of the present invention.

### BRIEF DESCRIPTION OF THE DRAWINGS

FIG. 1 is a synthetic scheme showing the synthesis of various analogs of 4-hydroxyisoleucine with SSS, SSR, SRS and SRR configuration.

FIG. 2 is a synthetic scheme showing the synthesis of of compounds 16 to 34.

FIG. 3 is a synthetic scheme showing the synthesis of compounds 35 to 38.

FIG. 4 is a synthetic scheme showing the synthesis of compounds 39 and 40.

FIG. 5 is a synthetic scheme showing the synthesis of compounds 41 to 62.

FIG. 6 is a synthetic scheme showing the synthesis of compounds 63 to 65.

FIG. 7 is a synthetic scheme showing the synthesis of compounds 66 to 69.

FIG. 8 is a synthetic scheme showing the synthesis of compounds 70 to 76.

FIG. 9 is a synthetic scheme showing the synthesis of compounds 77 and 78.

FIG. 10 is a synthetic scheme showing the synthesis of compounds 79 to 85.

FIG. 11 is a synthetic scheme showing the synthesis of compounds 86a to 102b.

FIG. 12 is a synthetic scheme showing the synthesis of compounds 103 to 123.

FIG. 13 is a synthetic scheme showing the synthesis of compounds 124 to 133.

FIG. 14 is a synthetic scheme showing the synthesis of two diastereoisomers and analog of (2S,3R,4S)-4-hydroxyisoleucine (compounds 12b & 13b).

FIGS. 15A and 15B are bar graphs showing that analogs of 4-Hydroxyisoleucine stimulate glucose uptake by differentiated 3T3-L1 adipocytes. The dashed lines delineate the baseline stimulation caused by Insulin (I).

FIGS. 16A and 16B are bar graphs showing glucose-dependent stimulation of insulin secretion in INS-1 cells by selected analogs of 4-Hydroxyisoleucine. The dashed lines represent the background insulin stimulating activity caused by 4.5 mM glucose (G).

FIGS. 17A, 17B, 17C, 17D and 17E are bar graphs showing glucose-dependent stimulation of insulin secretion in INS-1 cells by selected analogs of 4-Hydroxyisoleucine. The dashed lines represent the background insulin stimulating activity caused by 5 mM glucose (G) (FIG. 17A) or 4.5 mM glucose (G) (FIGS. 17B to 17E).

FIGS. 18A and 18B are bar graphs showing the glycemic response of mice following an OGTT performed after a single oral administration of selected analogs according to the invention.

FIGS. 19A, 19B, 19C, and 19D are bar graphs showing glycemic response of mice following an OGTT performed after 7 days (FIGS. 19A and 19D), 14 days (FIG. 19B) or 21 days (FIG. 19C) of treatment, respectively, after chronic oral administration of selected analogs according to the invention.

### DETAILED DESCRIPTION OF THE INVENTION

(2S,3R,4S)-4-hydroxyisoleucine is a compound that has been shown both to stimulate insulin secretion in a glucose dependent manner, and to decrease insulin resistance (see, e.g., U.S. Pat. No. 5,470,879; WO 01/15689; Broca et al., Am. J. Physiol. 277:E617-E623, 1999; Broca et al., Am. J. Physiol. Endocrinol. Metab. 287:E463-E471, 2004). The invention features chemical analogs, lactones, salts, metabolites and prodrugs of (2S,3R,4S)-4-hydroxyisoleucine, pharmaceutical compositions comprising the same and uses thereof for the prevention and/or treatment of disorders of carbohydrate or lipid metabolism, including diabetes mellitus (type 1 and type 2 diabetes), pre-diabetes and Metabolic Syndrome.

In order to provide an even clearer and more consistent understanding of the specification and the claims, including the scope given herein to such terms, the following definitions are provided:

A) Definitions

Unless otherwise stated, the following terms as used in the specification have the following meaning.

The term “4-hydroxyisoleucine” or “4-OH” as used herein generally refers to the compound 4-hydroxy-3-methylpentanoic acid and to configurational isomers thereof. More particularly it refers to the isomer (2S,3R,4S)-4-hydroxyisoleucine.

The terms “acyl” or “alkanoyl,” as used interchangeably herein, represent an alkyl group, as defined herein, or hydrogen attached to the parent molecular group through a carbonyl group, as defined herein, and is exemplified by formyl, acetyl, propionyl, butanoyl and the like. Exemplary unsubstituted acyl groups are of from 2 to 7 carbons.

The term “administration” or “administering” refers to a method of giving a dosage of a pharmaceutical composition to a mammal, where the method is, e.g., oral, subcutaneous, topical, intravenous, intraperitoneal, by inhalation, or intramuscular. The preferred method of administration can vary depending on various factors, e.g., the components of the pharmaceutical composition, site of the potential or actual disease, and severity of disease.

The term “alkenyl,” as used herein, represents monovalent straight or branched chain groups of, unless otherwise specified, from 2 to 12 carbons, such as, for example, 2 to 6 carbon atoms or 2 to 4 carbon atoms, containing one or more carbon-carbon double bonds and is exemplified by ethenyl, 1-propenyl, 2-propenyl, 2-methyl-i-propenyl, 1-butenyl, 2-butenyl and the like and may be optionally substituted with one, two, three or four substituents independently selected from the group consisting of: (1) alkoxy of one to six carbon atoms; (2) alkylsulfinyl of one to six carbon atoms; (3) alkylsulfonyl of one to six carbon atoms; (4) alkynyl of two to six carbon atoms; (5) amino; (6) aryl; (7) arylalkoxy, where the alkylene group is of one to six carbon atoms; (8) azido; (9) cycloalkyl of three to eight carbon atoms; (10) halo; (1 1) heterocyclyl; (12) (heterocycle)oxy; (13) (heterocycle)oyl; (14) hydroxyl; (15) hydroxyalkyl of one to 6 carbons; (16) N-protected amino; (17) nitro; (18) oxo or thiooxo; (19) perfluoroalkyl of 1 to 4 carbons; (20) perfluoroalkoxyl of 1 to 4 carbons; (21) spiroalkyl of three to eight carbon atoms; (22) thioalkoxy of one to six carbon atoms; (23) thiol; (24) OC(O)R^{A}, where R^{A} is selected from the group consisting of (a) substituted or unsubstituted C₁₋₆ alkyl, (b) substituted or unsubstituted C₆ or C₁₀ aryl, (c) substituted or unsubstituted C₇₋₁₆ arylalkyl, where the alkylene group is of one to six carbon atoms, (d) substituted or unsubstituted C₁₋₉ heterocyclyl, and (e) substituted or unsubstituted C₂₋₁₅ heterocyclylalkyl, where the alkylene group is of one to six carbon atoms; (25) C(O)R^{B}, where R^{B} is selected from the group consisting of (a) hydrogen, (b) substituted or unsubstituted C₁₋₆alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (f) substituted or unsubstituted C₂₋₁₅ heterocyclylalkyl, where the alkylene group is of one to six carbon atoms; (26) CO₂R^{B}, where R^{B} is selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (f) substituted or unsubstituted C₂₋₁₅ heterocyclylalkyl, where the alkylene group is of one to six carbon atoms; (27) C(O)NR^{C}R^{D}, where each of R^{C} and R^{D} is, independently, selected from the group consisting of (a) hydrogen, (b) alkyl, (c) aryl and (d) arylalkyl, where the alkylene group is of one to six carbon atoms; (28) S(O)R^{E}, where R^{E} is selected from the group consisting of (a) alkyl, (b) aryl, (c) arylalkyl, where the alkylene group is of one to six carbon atoms, and hydroxyl; (29) S(O)₂R^{E}, where R^{E} is selected from the group consisting of (a) alkyl, (b) aryl, (c) arylalkyl, where the alkylene group is of one to six carbon atoms, and hydroxyl; (30) S(O)₂NR^{F}R^{G}, where each of R^{F} and R^{G} is, independently, selected from the group consisting of (a) hydrogen, (b) alkyl, (c) aryl and (d) arylalkyl, where the alkylene group is of one to six carbon atoms; and (31) —NR^{H}R^{I}, where each of R^{H} and R^{I} is, independently, selected from the group consisting of (a) hydrogen; (b) an N-protecting group; (c) alkyl of one to six carbon atoms; (d) alkenyl of two to six carbon atoms; (e) alkynyl of two to six carbon atoms; (f) aryl; (g) arylalkyl, where the alkylene group is of one to six carbon atoms; (h) cycloalkyl of three to eight carbon atoms, (i) alkcycloalkyl, where the cycloalkyl group is of three to eight carbon atoms, and the alkylene group is of one to ten carbon atoms, (j) alkanoyl of one to six carbon atoms, (k) aryloyl of 6 to 10 carbon atoms, (l) alkylsulfonyl of one to six carbon atoms, and (m) arylsulfonyl of 6 to 10 carbons atoms, with the proviso that no two groups are bound to the nitrogen atom through a carbonyl group or a sulfonyl group.

The terms “alkoxy” or “alkyloxy,” as used interchangeably herein, represent an alkyl group attached to the parent molecular group through an oxygen atom. Exemplary unsubstituted alkoxy groups are of from 1 to 6 carbons.

The term “alkyl” or “alk” as used herein, represents a monovalent group derived from a straight or branched chain saturated hydrocarbon of, unless otherwise specified, from 1 to 6 carbons and is exemplified by methyl, ethyl, n- and iso-propyl, n-, sec-, iso- and tert-butyl, neopentyl and the like and may be optionally substituted with one, two, three or, in the case of alkyl groups of two carbons or more, four substituents independently selected from the group consisting of: (1) alkoxy of one to six carbon atoms; (2) alkylsulfinyl of one to six carbon atoms; (3) alkylsulfonyl of one to six carbon atoms; (4) alkynyl of two to six carbon atoms; (5) amino; (6) aryl; (7) arylalkoxy, where the alkylene group is of one to six carbon atoms; (8) azido; (9) cycloalkyl of three to eight carbon atoms; (10) halo; (11) heterocyclyl; (12) (heterocycle)oxy; (13) (heterocycle)oyl; (14) hydroxyl; (15) hydroxyalkyl of one to 6 carbons; (16) N-protected amino; (17) nitro; (18) oxo or thiooxo; (19) perfluoroalkyl of 1 to 4 carbons; (20) perfluoroalkoxyl of 1 to 4 carbons; (21) spiroalkyl of three to eight carbon atoms; (22) thioalkoxy of one to six carbon atoms; (23) thiol; (24) OC(O)R^{A}, where R^{A} is selected from the group consisting of (a) substituted or unsubstituted C₁₋₆ alkyl, (b) substituted or unsubstituted C₆ or C₁₀ aryl, (c) substituted or unsubstituted C₇₋₁₆ arylalkyl, where the alkylene group is of one to six carbon atoms, (d) substituted or unsubstituted C₁₋₉ heterocyclyl, and (e) substituted or unsubstituted C₂₋₁₅ heterocyclylalkyl, where the alkylene group is of one to six carbon atoms; (25) C(O)R^{B}, where R^{B} is selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (e substituted or unsubstituted C₂₋₁₅ heterocyclylalkyl, where the alkylene group is of one to six carbon atoms; (26) CO₂R^{B}, where R^{B} is selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (f) substituted or unsubstituted C₂₋₁₅ heterocyclylalkyl, where the alkylene group is of one to six carbon atoms; (27) C(O)NR^{C}R^{D}, where each of R^{C} and R^{D} is, independently, selected from the group consisting of (a) hydrogen, (b) alkyl, (c) aryl and (d) arylalkyl, where the alkylene group is of one to six carbon atoms; (28) S(O)R^{E}, where R^{E} is selected from the group consisting of (a) alkyl, (b) aryl, (c) arylalkyl, where the alkylene group is of one to six carbon atoms, and hydroxyl; (29) S(O)₂R^{E}, where R^{E} is selected from the group consisting of (a) alkyl, (b) aryl, (c) arylalkyl, where the alkylene group is of one to six carbon atoms, and hydroxyl; (30) S(O)₂NR^{F}R^{G}, where each of R^{F} and R^{G} is, independently, selected from the group consisting of (a) hydrogen, (b) alkyl, (c) aryl and (d) arylalkyl, where the alkylene group is of one to six carbon atoms; and (31) —NR^{H}R^{I}, where each of R^{H} and R^{I} is, independently, selected from the group consisting of (a) hydrogen; (b) an N-protecting group; (c) alkyl of one to six carbon atoms; (d) alkenyl of two to six carbon atoms; (e) alkynyl of two to six carbon atoms; (f aryl; (g) arylalkyl, where the alkylene group is of one to six carbon atoms; (h) cycloalkyl of three to eight carbon atoms, (i) alkcycloalkyl, where the cycloalkyl group is of three to eight carbon atoms, and the alkylene group is of one to ten carbon atoms, (j) alkanoyl of one to six carbon atoms, (k) aryloyl of 6 to 10 carbon atoms, (l) alkylsulfonyl of one to six carbon atoms, and (m) arylsulfonyl of 6 to 10 carbons atoms, with the proviso that no two groups are bound to the nitrogen atom through a carbonyl group or a sulfonyl group.

The term “alkylene,” as used herein, represents a saturated divalent hydrocarbon group derived from a straight or branched chain saturated hydrocarbon by the removal of two hydrogen atoms, and is exemplified by methylene, ethylene, isopropylene and the like.

The term “alkylsulfinyl,” as used herein, represents an alkyl group attached to the parent molecular group through an S(O) group. Exemplary unsubstituted alkylsulfinyl groups are of from 1 to 6 carbons.

The term “alkylsulfonyl,” as used herein, represents an alkyl group attached to the parent molecular group through an S(O)₂ group. Exemplary unsubstituted alkylsulfonyl groups are of from 1 to 6 carbons.

The term “arylsulfonyl,” as used herein, represents an aryl group attached to the parent molecular group through an S(O)₂ group.

The term “alkylthio,” as used herein, represents an alkyl group attached to the parent molecular group through a sulfur atom. Exemplary unsubstituted alkylthio groups are of from 1 to 6 carbons.

The term “alkynyl,” as used herein, represents monovalent straight or branched chain groups of from two to six carbon atoms containing a carbon-carbon triple bond and is exemplified by ethynyl, 1-propynyl, and the like and may be optionally substituted with one, two, three or four substituents independently selected from the group consisting of: (1) alkoxy of one to six carbon atoms; (2) alkylsulfinyl of one to six carbon atoms; (3) alkylsulfonyl of one to six carbon atoms; (4) alkynyl of two to six carbon atoms; (5) amino; (6) aryl; (7) arylalkoxy, where the alkylene group is of one to six carbon atoms; (8) azido; (9) cycloalkyl of three to eight carbon atoms; (10) halo; (11) heterocyclyl; (12) (heterocycle)oxy; (13) (heterocycle)oyl; (14) hydroxyl; (15) hydroxyalkyl of one to 6 carbons; (16) N-protected amino; (17) nitro; (18) oxo or thiooxo; (19) perfluoroalkyl of 1 to 4 carbons; (20) perfluoroalkoxyl of 1 to 4 carbons; (21) spiroalkyl of three to eight carbon atoms; (22) thioalkoxy of one to six carbon atoms; (23) thiol; (24) OC(O)R^{A}, where R^{A} is selected from the group consisting of (a) substituted or unsubstituted C₁₋₆ alkyl, (b) substituted or unsubstituted C₆ or C₁₀ aryl, (c) substituted or unsubstituted C₇₋₁₆ arylalkyl, where the alkylene group is of one to six carbon atoms, (d) substituted or unsubstituted C₁₋₉ heterocyclyl, and (e) substituted or unsubstituted C₂₋₁₅ heterocyclylalkyl, where the alkylene group is of one to six carbon atoms; (25) C(O)R^{B}, where R^{B} is selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (f) substituted or unsubstituted C₂₋₁₅ heterocyclylalkyl, where the alkylene group is of one to six carbon atoms; (26) CO₂R^{B}, where R^{B} is selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (f) substituted or unsubstituted C₂₋₁₅ heterocyclylalkyl, where the alkylene group is of one to six carbon atoms; (27) C(O)NR^{C}R^{D}, where each of R^{C} and R^{D} is, independently, selected from the group consisting of (a) hydrogen, (b) alkyl, (c) aryl and (d) arylalkyl, where the alkylene group is of one to six carbon atoms; (28) S(O)R^{E}, where R^{E} is selected from the group consisting of (a) alkyl, (b) aryl, (c) arylalkyl, where the alkylene group is of one to six carbon atoms, and hydroxyl; (29) S(O)₂R^{E}, where R^{E} is selected from the group consisting of (a) alkyl, (b) aryl, (c) arylalkyl, where the alkylene group is of one to six carbon atoms, and hydroxyl; (30) S(O)₂NR^{F}R^{G}, where each of R^{F} and R^{G} is, independently, selected from the group consisting of (a) hydrogen, (b) alkyl, (c) aryl and (d) arylalkyl, where the alkylene group is of one to six carbon atoms; and (31) —NR^{H}R^{I}, where each of R^{H} and R^{I} is, independently, selected from the group consisting of (a) hydrogen; (b) an N-protecting group; (c) alkyl of one to six carbon atoms; (d) alkenyl of two to six carbon atoms; (e) alkynyl of two to six carbon atoms; (f) aryl; (g) arylalkyl, where the alkylene group is of one to six carbon atoms; (h) cycloalkyl of three to eight carbon atoms, (i) alkcycloalkyl, where the cycloalkyl group is of three to eight carbon atoms, and the alkylene group is of one to ten carbon atoms, (j) alkanoyl of one to six carbon atoms, (k) aryloyl of 6 to 10 carbon atoms, (I) alkylsulfonyl of one to six carbon atoms, and (m) arylsulfonyl of 6 to 10 carbons atoms, with the proviso that no two groups are bound to the nitrogen atom through a carbonyl group or a sulfonyl group.

The term “alpha-amino acid residue” as used herein, represents a N(R^{A})C(R^{B})(R^{C})C(O)— linkage, where R^{A} is selected from the group consisting of (a) hydrogen, (b) alkyl, (c) aryl and (d) arylalkyl, as defined herein; and each of R^{B} and R^{C} is, independently, selected from the group consisting of: (a) hydrogen, (b) optionally substituted alkyl, (c) optionally substituted cycloalkyl, (d) optionally substituted aryl, (e) optionally substituted arylalkyl, (f) optionally substituted heterocyclyl, and (g) optionally substituted heterocyclylalkyl, each of which is as defined herein. For natural amino acids, R^{B} is H and R^{C} corresponds to those side chains of natural amino acids found in nature, or their antipodal configurations. Exemplary natural amino acids include alanine, cysteine, aspartic acid, glutamic acid, phenylalanine, glycine, histidine, isoleucine, lysine, leucine, methionine, aspartamine, ornithine, proline, glutamine, arginine, serine, threonine, valine, tryptophan, and tyrosine, each of which, except glycine, as their D- or L-form. As used herein, for the most part, the names of naturally-occurring amino acids and acylamino residues used herein follow the naming conventions suggested by the IUPAC Commission on the Nomenclature of Organic Chemistry and the IUPAC-IUB Commission on Biochemical Nomenclature as set out in Nomenclature of α-Amino Acids (Recommendations, 1974), Biochemistry 14 (2), 1975. The present invention also contemplates non-naturally occurring (i.e., unnatural) amino acid residues in their D- or L-form such as, for example, homophenylalanine, phenylglycine, cyclohexylglycine, cyclohexylalanine, cyclopentyl alanine, cyclobutylalanine, cyclopropylalanine, cyclohexylglycine, norvaline, norleucine, thiazoylalanine (2-, 4- and 5-substituted), pyridylalanine (2-, 3- and 4-isomers), naphthalalanine (1- and 2-isomers) and the like. Stereochemistry is as designated by convention, where a bold bond indicates that the substituent is oriented toward the viewer (away from the page) and a dashed bond indicates that the substituent is oriented away from the viewer (into the page). If no stereochemical designation is made, it is to be assumed that the structure definition includes both stereochemical possibilities.

The term “amino” as used herein, represents an —NH₂ group.

The term “aminoalkyl” represents an amino group attached to the parent molecular group through an alkyl group.

The terms “analog(s) of 4-hydroxyisoleucine”, “analog(s)s of 4-OH”, “analog(s) of the invention” or “compound(s)s of the invention” as used herein, refers to the compounds of any of Formulae I, II and/or III as defined herein and include pharmaceutically acceptable lactones, salts, crystal forms, metabolites, solvates, esters and prodrugs of the compounds Formulae I, II and III.

The term “aryl” as used herein, represents a mono- or bicyclic carbocyclic ring system having one or two aromatic rings and is exemplified by phenyl, naphthyl, 1,2-dihydronaphthyl, 1,2,3,4-tetrahydronaphthyl, fluorenyl, indanyl, indenyl and the like and may be optionally substituted with one, two, three, four or five substituents independently selected from the group consisting of: (1) alkanoyl of one to six carbon atoms; (2) alkyl of one to six carbon atoms; (3) alkoxy of one to six carbon atoms; (4) alkoxyalkyl, where the alkyl and alkylene groups are independently of one to six carbon atoms; (5) alkylsulfinyl of one to six carbon atoms; (6) alkylsulfinylalkyl, where the alkyl and alkylene groups are independently of one to six carbon atoms; (7) alkylsulfonyl of one to six carbon atoms; (8) alkylsulfonylalkyl, where the alkyl and alkylene groups are independently of one to six carbon atoms; (9) aryl; (10) arylalkyl, where the alkyl group is of one to six carbon atoms; (11) amino; (12) aminoalkyl of one to six carbon atoms; (13) aryl; (14) arylalkyl, where the alkylene group is of one to six carbon atoms; (15) aryloyl; (16) azido; (17) azidoalkyl of one to six carbon atoms; (18) carboxaldehyde; (19) (carboxaldehyde)alkyl, where the alkylene group is of one to six carbon atoms; (20) cycloalkyl of three to eight carbon atoms; (21) alkcycloalkyl, where the cycloalkyl group is of three to eight carbon atoms and the alkylene group is of one to ten carbon atoms; (22) halo; (23) haloalkyl of one to six carbon atoms; (24) heterocyclyl; (25) (heterocyclyl)oxy; (26) (heterocyclyl)oyl; (27) hydroxy; (28) hydroxyalkyl of one to six carbon atoms; (29) nitro; (30) nitroalkyl of one to six carbon atoms; (31) N-protected amino; (32) N-protected aminoalkyl, where the alkylene group is of one to six carbon atoms; (33) oxo; (34) thioalkoxy of one to six carbon atoms; (35) thioalkoxyalkyl, where the alkyl and alkylene groups are independently of one to six carbon atoms; (36) (CH₂)_{q}CO₂R^{A}, where q is an integer of from zero to four and R^{A} is selected from the group consisting of (a) alkyl, (b) aryl and (c) arylalkyl, where the alkylene group is of one to six carbon atoms; (37) (CH₂)_{q}C(O)NR^{B}R^{C}, where R^{B} and R^{C} are independently selected from the group consisting of (a) hydrogen, (b) alkyl, (c) aryl and (d) arylalkyl, where the alkylene group is of one to six carbon atoms; (38) (CH₂)_{q}S(O)₂R^{D}, where R^{D} is selected from the group consisting of (a) alkyl, (b) aryl and (c) arylalkyl, where the alkylene group is of one to six carbon atoms; (39) (CH₂)_{q}S(O)₂NR^{E}R^{F}, where each of R^{E} and R^{F} is, independently, selected from the group consisting of (a) hydrogen, (b) alkyl, (c) aryl and (d) arylalkyl, where the alkylene group is of one to six carbon atoms; (40) (CH₂)_{q}NR^{G}R^{H}, where each of R^{G} and R^{H} is, independently, selected from the group consisting of (a) hydrogen; (b) an N-protecting group; (c) alkyl of one to six carbon atoms; (d) alkenyl of two to six carbon atoms; (e) alkynyl of two to six carbon atoms; (f) aryl; (g) arylalkyl, where the alkylene group is of one to six carbon atoms; (h) cycloalkyl of three to eight carbon atoms and (i) alkcycloalkyl, where the cycloalkyl group is of three to eight carbon atoms, and the alkylene group is of one to ten carbon atoms, with the proviso that no two groups are bound to the nitrogen atom through a carbonyl group or a sulfonyl group; (41) oxo; (42) thiol; (43) perfluoroalkyl; (44) perfluoroalkoxy; (45) aryloxy; (46) cycloalkoxy; (47) cycloalkylalkoxy; and (48) arylalkoxy.

The term “alkaryl” represents an aryl group attached to the parent molecular group through an alkyl group. Exemplary unsubstituted arylalkyl groups are of from 7 to 16 carbons.

The term “alkheterocyclyl” represents a heterocyclic group attached to the parent molecular group through an alkyl group. Exemplary unsubstituted alkheterocyclyl groups are of from 2 to 10 carbons.

The term “alkylsulfinylalkyl” represents an alkylsulfinyl group attached to the parent molecular group through an alkyl group.

The term “alkylsulfonylalkyl” represents represents an alkylsulfonyl group attached to the parent molecular group through an alkyl group.

The term “aryloxy,” as used herein, represents an aryl group that is attached to the parent molecular group through an oxygen atom. Exemplary unsubstituted aryloxy groups are of 6 or 10 carbons.

The terms “aryloyl” or “aroyl” as used interchangeably herein, represent an aryl group that is attached to the parent molecular group through a carbonyl group. Exemplary unsubstituted aryloxycarbonyl groups are of 7 or 11 carbons.

The term “azido” represents an N₃ group, which can also be represented as N═N═N.

The term “azidoalkyl” represents an azido group attached to the parent molecular group through an alkyl group.

The term “carbonyl” as used herein, represents a C(O) group, which can also be represented as C═O.

The term “carboxyaldehyde” represents a CHO group.

The term “carboxaldehydealkyl” represents a carboxyaldehyde group attached to the parent molecular group through an alkyl group.

The term “carboxy” or “carboxyl,” as used interchangeably herein, represents a CO₂H group.

The terms “carboxy protecting group” or “carboxyl protecting group” as used herein, represent those groups intended to protect a CO₂H group against undesirable reactions during synthetic procedures. Commonly used carboxy-protecting groups are disclosed in Greene, “Protective Groups In Organic Synthesis, 3^{rd} Edition” (John Wiley & Sons, New York, 1999), which is incorporated herein by reference.

The term “configurational isomer of 4-hydroxyisoleucine” means one of the following compounds: (2S,3R,4S)-, (2R,3S,4S)-, (2S,3S,4S)-, (2R,3R,4S)-m (2S,3R,4R)-, (2S,3S,4R)-, (2R,3S,4R)-, or (2R,3R,4R)-4-hydroxyisoleucine.

The term “cycloalkyl” as used herein represents a monovalent saturated or unsaturated non-aromatic cyclic hydrocarbon group of from three to eight carbons, unless otherwise specified, and is exemplified by cyclopropyl, cyclobutyl, cyclopentyl, cyclohexyl, cycloheptyl, bicyclo[2.2.1.]heptyl and the like. The cycloalkyl groups of this invention can be optionally substituted with (1) alkanoyl of one to six carbon atoms; (2) alkyl of one to six carbon atoms; (3) alkoxy of one to six carbon atoms; (4) alkoxyalkyl, where the alkyl and alkylene groups are independently of one to six carbon atoms; (5) alkylsulfinyl of one to six carbon atoms; (6) alkylsulfinylalkyl, where the alkyl and alkylene groups are independently of one to six carbon atoms; (7) alkylsulfonyl of one to six carbon atoms; (8) alkylsulfonylalkyl, where the alkyl and alkylene groups are independently of one to six carbon atoms; (9) aryl; (10) arylalkyl, where the alkyl group is of one to six carbon atoms; (11) amino; (12) aminoalkyl of one to six carbon atoms; (13) aryl; (14) arylalkyl, where the alkylene group is of one to six carbon atoms; (15) aryloyl; (16) azido; (17) azidoalkyl of one to six carbon atoms; (18) carboxaldehyde; (19) (carboxaldehyde)alkyl, where the alkylene group is of one to six carbon atoms; (20) cycloalkyl of three to eight carbon atoms; (21) alkcycloalkyl, where the cycloalkyl group is of three to eight carbon atoms and the alkylene group is of one to ten carbon atoms; (22) halo; (23) haloalkyl of one to six carbon atoms; (24) heterocyclyl; (25) (heterocyclyl)oxy; (26) (heterocyclyl)oyl; (27) hydroxy; (28) hydroxyalkyl of one to six carbon atoms; (29) nitro; (30) nitroalkyl of one to six carbon atoms; (31) N-protected amino; (32) N-protected aminoalkyl, where the alkylene group is of one to six carbon atoms; (33) oxo; (34) thioalkoxy of one to six carbon atoms; (35) thioalkoxyalkyl, where the alkyl and alkylene groups are independently of one to six carbon atoms; (36) (CH₂)_{q}CO₂R^{A}, where q is an integer of from zero to four and R^{A} is selected from the group consisting of (a) alkyl, (b) aryl and (c) arylalkyl, where the alkylene group is of one to six carbon atoms; (37) (CH₂)_{q}C(O)NR^{B}R^{C}, where each of R^{B} and R^{C} is, independently, selected from the group consisting of (a) hydrogen, (b) alkyl, (c) aryl and (d) arylalkyl, where the alkylene group is of one to six carbon atoms; (38) (CH₂)_{q}S(O)₂R^{D}, where R^{D} is selected from the group consisting of (a) alkyl, (b) aryl and (c) arylalkyl, where the alkylene group is of one to six carbon atoms; (39) (CH₂)_{q}S(O)₂NR^{E}R^{F}, where each of R^{E} and R^{F} is, independently, selected from the group consisting of (a) hydrogen, (b) alkyl, (c) aryl and (d) arylalkyl, where the alkylene group is of one to six carbon atoms; (40) (CH₂)_{q}NR^{G}R^{H}, where each of R^{G} and R^{H} is, independently, selected from the group consisting of (a) hydrogen; (b) an N-protecting group; (c) alkyl of one to six carbon atoms; (d) alkenyl of two to six carbon atoms; (e) alkynyl of two to six carbon atoms; (f aryl; (g) arylalkyl, where the alkylene group is of one to six carbon atoms; (h) cycloalkyl of three to eight carbon atoms and (i) alkcycloalkyl, where the cycloalkyl group is of three to eight carbon atoms, and the alkylene group is of one to ten carbon atoms, with the proviso that no two groups are bound to the nitrogen atom through a carbonyl group or a sulfonyl group; (41) oxo; (42) thiol; (43) perfluoroalkyl; (44) perfluoroalkoxy; (45) aryloxy; (46) cycloalkoxy; (47) cycloalkylalkoxy; and (48) arylalkoxy.

By “disorder of carbohydrate metabolism” is meant a metabolic disorder in which the subject having the disorder cannot properly metabolize sugars. Examples of such disorders include, for example, diabetes mellitus (type 1 and type 2), pre-diabetes, hyperglycemia, impaired glucose tolerance, Metabolic Syndrome, glucosuria, diabetic neuropathy and nephropathy, obesity, and eating disorders.

By “disorder of lipid metabolism” is meant a metabolic disorder in which the subject having the disorder cannot properly metabolize, distribute and/or store fat. Examples of such disorders include, but are not limited to type 2 diabetes, pre-diabetes, and Metabolic Syndrome.

By “effective amount” is meant the amount of a compound required to treat or prevent a disorder of carbohydrate or lipid metabolism, such as, for example, diabetes and Metabolic Syndrome. The effective amount of active compound(s) used to practice the present invention for therapeutic or prophylactic treatment of conditions caused by or contributed to by a disorder of carbohydrate or lipid metabolism varies depending upon the manner of administration, and the age, body weight, and general health of the subject. Ultimately, the attending physician or veterinarian will decide the appropriate amount and dosage regimen. An effective amount can also be that which provides some amelioration of one or more symptoms of the disorder or decreases the likelihood of incidence of the disorder.

The term “halogen” or “halo” as used interchangeably herein, represents F, Cl, Br and I.

The term “haloalkyl” represents a halo group, as defined herein, attached to the parent molecular group through an alkyl group.

The term “heteroaryl,” as used herein, represents that subset of heterocycles, as defined herein, which are aromatic: i.e., they contain 4n+2 pi electrons within the mono- or multicyclic ring system. Exemplary unsubstituted heteroaryl groups are of from 1 to 9 carbons.

The terms “heterocycle” or “heterocyclyl” as used interchangeably herein represent a 5-, 6- or 7-membered ring, unless otherwise specified, containing one, two, three, or four heteroatoms independently selected from the group consisting of nitrogen, oxygen, and sulfur. The 5-membered ring has zero to two double bonds and the 6- and 7-membered rings have zero to three double bonds. The term “heterocycle” also includes bicyclic, tricyclic, and tetracyclic groups in which any of the above heterocyclic rings is fused to one or two rings independently selected from the group consisting of an aryl ring, a cyclohexane ring, a cyclohexene ring, a cyclopentane ring, a cyclopentene ring and another monocyclic heterocyclic ring such as indolyl, quinolyl, isoquinolyl, tetrahydroquinolyl, benzofuryl, benzothienyl and the like. Heterocyclics include pyrrolyl, pyrrolinyl, pyrrolidinyl, pyrazolyl, pyrazolinyl, pyrazolidinyl, imidazolyl, imidazolinyl, imidazolidinyl, pyridyl, piperidinyl, homopiperidinyl, pyrazinyl, piperazinyl, pyrimidinyl, pyridazinyl, oxazolyl, oxazolidinyl, isoxazolyl, isoxazolidiniyl, morpholinyl, thiomorpholinyl, thiazolyl, thiazolidinyl, isothiazolyl, isothiazolidinyl, indolyl, quinolinyl, isoquinolinyl, benzimidazolyl, benzothiazolyl, benzoxazolyl, furyl, thienyl, thiazolidinyl, isothiazolyl, isoindazoyl, triazolyl, tetrazolyl, oxadiazolyl, uricyl, thiadiazolyl, pyrimidyl, tetrahydrofuranyl, dihydrofuranyl, tetrahydrothienyl, dihydrothienyl, dihydroinidolyl, tetrahydroquinolyl, tetrahydroisoquinolyl, pyranyl, dihydropyranyl, dithiazolyl, benzofuranyl, benzothienyl and the like. Heterocyclic groups also include compounds of the formula

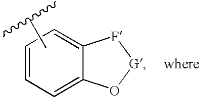

F′ is selected from the group consisting of CH₂, CH₂O and O, and G′ is selected from the group consisting of C(O) and (C(R″)(R′″)), where each of R″ and R′″ is, independently, selected from the group consisting of hydrogen or alkyl of one to four carbon atoms, and v is one to three and includes groups such as 1,3-benzodioxolyl, 1,4-benzodioxanyl and the like. Any of the heterocycle groups mentioned herein may be optionally substituted with one, two, three, four or five substituents independently selected from the group consisting of: (1) alkanoyl of one to six carbon atoms; (2) alkyl of one to six carbon atoms; (3) alkoxy of one to six carbon atoms; (4) alkoxyalkyl, where the alkyl and alkylene groups are independently of one to six carbon atoms; (5) alkylsulfinyl of one to six carbon atoms; (6) alkylsulfinylalkyl, where the alkyl and alkylene groups are independently of one to six carbon atoms; (7) alkylsulfonyl of one to six carbon atoms; (8) alkylsulfonylalkyl, where the alkyl and alkylene groups are independently of one to six carbon atoms; (9) aryl; (10) arylalkyl, where the alkyl group is of one to six carbon atoms; (11) amino; (12) aminoalkyl of one to six carbon atoms; (13) aryl; (14) arylalkyl, where the alkylene group is of one to six carbon atoms; (15) aryloyl; (16) azido; (17) azidoalkyl of one to six carbon atoms; (18) carboxaldehyde; (19) (carboxaldehyde)alkyl, where the alkylene group is of one to six carbon atoms; (20) cycloalkyl of three to eight carbon atoms; (21) alkcycloalkyl, where the cycloalkyl group is of three to eight carbon atoms and the alkylene group is of one to ten carbon atoms; (22) halo; (23) haloalkyl of one to six carbon atoms; (24) heterocycle; (25) (heterocycle)oxy; (26) (heterocycle)oyl; (27) hydroxy; (28) hydroxyalkyl of one to six carbon atoms; (29) nitro; (30) nitroalkyl of one to six carbon atoms; (31) N-protected amino; (32) N-protected aminoalkyl, where the alkylene group is of one to six carbon atoms; (33) oxo; (34) thioalkoxy of one to six carbon atoms; (35) thioalkoxyalkyl, where the alkyl and alkylene groups are independently of one to six carbon atoms; (36) (CH₂)_{q}CO₂R^{A}, where q is an integer of from zero to four and R^{A} is selected from the group consisting of (a) alkyl, (b) aryl and (c) arylalkyl, where the alkylene group is of one to six carbon atoms; (37) (CH₂)_{q}C(O)NR^{B}R^{C}, where each of R^{B} and R^{C} is, independently, selected from the group consisting of (a) hydrogen, (b) alkyl, (c) aryl and (d) arylalkyl, where the alkylene group is of one to six carbon atoms; (38) (CH₂)_{q}S(O)₂R^{D}, where R^{D} is selected from the group consisting of (a) alkyl, (b) aryl and (c) arylalkyl, where the alkylene group is of one to six carbon atoms; (39) (CH₂)_{q}S(O)₂NR^{E}R^{F}, where each of R^{E} and R^{F} is, independently, selected from the group consisting of (a) hydrogen, (b) alkyl, (c) aryl and (d) arylalkyl, where the alkylene group is of one to six carbon atoms; (40) (CH₂)_{q}NR^{G}R^{H}, where each of R^{G} and R^{H} is, independently, selected from the group consisting of (a) hydrogen; (b) an N-protecting group; (c) alkyl of one to six carbon atoms; (d) alkenyl of two to six carbon atoms; (e) alkynyl of two to six carbon atoms; (e aryl; (g) arylalkyl, where the alkylene group is of one to six carbon atoms; (h) cycloalkyl of three to eight carbon atoms and (i) alkcycloalkyl, where the cycloalkyl group is of three to eight carbon atoms, and the alkylene group is of one to ten carbon atoms, with the proviso that no two groups are bound to the nitrogen atom through a carbonyl group or a sulfonyl group; (41) oxo; (42) thiol; (43) perfluoroalkyl; (44) perfluoroalkoxy; (45) aryloxy; (46) cycloalkoxy; (47) cycloalkylalkoxy; and (48) arylalkoxy.

The terms “heterocyclyloxy” or “(heterocycle)oxy” as used interchangeably herein, represents a heterocycle group, as defined herein, attached to the parent molecular group through an oxygen atom. Exemplary unsubstituted heterocyclyloxy groups are of from 1 to 9 carbons.

The term “heterocyclyloyl” or “(heterocycle)oyl” as used interchangeably herein, represents a heterocycle group, as defined herein, attached to the parent molecular group through a carbonyl group. Exemplary unsubstituted heterocyclyloyl groups are of from 2 to 10 carbons.

The term “hydroxy” or “hydroxyl,” as used interchangeably herein, represents an —OH group.

The term “hydroxyalkyl” as used herein, represents an alkyl group, as defined herein, substituted by one to three hydroxy groups, with the proviso that no more than one hydroxy group may be attached to a single carbon atom of the alkyl group and is exemplified by hydroxymethyl, dihydroxypropyl and the like.

The term “N-protected amino” as used herein, refers to an amino group, as defined herein, to which is attached an N-protecting or nitrogen-protecting group, as defined herein.

The terms “N-protecting group” or “nitrogen protecting group” as used herein, represent those groups intended to protect an amino group against undesirable reactions during synthetic procedures. Commonly used N-protecting groups are disclosed in Greene, “Protective Groups In Organic Synthesis, 3^{rd} Edition” (John Wiley & Sons, New York, 1999), which is incorporated herein by reference. N-protecting groups comprise acyl, aroyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, 4-nitrobenzoyl and chiral auxiliaries such as protected or unprotected D, L or D, L-amino acids such as alanine, leucine, phenylalanine and the like; sulfonyl groups such as benzenesulfonyl, p-toluenesulfonyl and the like; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyl oxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, phenylthiocarbonyl and the like, arylalkyl groups such as benzyl, triphenylmethyl, benzyloxymethyl and the like and silyl groups such as trimethylsilyl and the like. Preferred N-protecting groups are formyl, acetyl, benzoyl, pivaloyl, t-butylacetyl, alanyl, phenylsulfonyl, benzyl, t-butyloxycarbonyl (Boc) and benzyloxycarbonyl (Cbz).

The term “nitro” as used herein, represents an —NO₂ group.

The term “nitroalkyl” represents a nitro group attached to the parent molecular group through an alkyl group. The term “non-vicinal O, S, or NR′” is meant an oxygen, sulfur, or nitrogen heteroatom substituent in a linkage, where the heteroatom substituent does not form a bond to a saturated carbon that is bonded to another heteroatom.

The term “oxo” as used herein, represents ═O.

The term “perfluoroalkyl” as used herein, represents an alkyl group, as defined herein, where each hydrogen radical bound to the alkyl group has been replaced by a fluoride radical. Perfluoroalkyl groups are exemplified by trifluoromethyl, pentafluoroethyl, and the like.

The term “perfluoroalkoxy” represents as used herein, represents an alkoxy group, as defined herein, where each hydrogen radical bound to the alkoxy group has been replaced by a fluoride radical. The term “pharmaceutically acceptable salt” as use herein, represents those salts which are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, S. M. Berge et al. describe pharmaceutically acceptable salts in detail in J. Pharmaceutical Sciences 66:1-19, 1977. The salts can be prepared in situ during the final isolation and purification of the compounds of the invention or separately by reacting the free base group with a suitable organic acid. Representative acid addition salts include acetate, adipate, alginate, ascorbate, aspartate, benzenesulfonate, benzoate, bisulfate, borate, butyrate, camphorate, camphersulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts and the like. Representative alkali or alkaline earth metal salts include sodium, lithium, potassium, calcium, magnesium and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine and the like.

The term “pharmaceutically acceptable ester” as used herein, represents esters that hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl group preferably has not more than 6 carbon atoms. Examples of particular esters include formates, acetates, propionates, butyates, acrylates and ethylsuccinates.

The term “prodrug” as used herein, represents compounds that are rapidly transformed in vivo to a parent compound of the above formula, for example, by hydrolysis in blood. A thorough discussion is provided in T. Higuchi and V. Stella, Pro-drugs as Novel Delivery Systems, Vol. 14 of the A.C.S. Symposium Series, Edward B. Roche, ed., Bioreversible Carriers in Drug Design, American Pharmaceutical Association and Pergamon Press, 1987, and Judkins et al., Synthetic Communications 26(23):4351-4367, 1996, each of which is incorporated herein by reference.

Prodrugs of an analog of the invention having Formulae (I), (II) or (III) are prepared by modifying functional groups present in any of the compounds of Formulae (I), (II) or (III) in such a way that the modifications may be cleaved in vivo to release the parent analog. Prodrugs include compounds of Formulae (I), (II) or (III) wherein a hydroxy, amino, or sulfhydryl group in any of said Formulae is bonded to any group that may be cleaved in vivo to regenerate the free hydroxyl, amino, or sulfhydryl group, respectively. Examples of prodrugs include, but are not limited to esters (e.g., acetate, formate, and benzoate derivatives), carbamates (e.g., N,N-dimethylaminocarbonyl) of hydroxy functional groups in compounds of Formulae (I), (II) or (III), and the like.

The term “pharmaceutically acceptable prodrugs” as used herein, represents those prodrugs of the compounds of the present invention which are, within the scope of sound medical judgment, suitable for use in contact with the tissues of humans and animals without undue toxicity, irritation, allergic response, and the like, commensurate with a reasonable benefit/risk ratio, and effective for their intended use, as well as the zwitterionic forms, where possible, of the compounds of the invention.

A “pharmaceutically acceptable active metabolite” is intended to mean a pharmacologically active product produced through metabolism in the body of a compound of Formulae (I), (II) or (III) as defined herein.

A “pharmaceutically acceptable solvate” is intended to mean a solvate that retains the biological effectiveness and properties of the biologically active components of compounds of Formulae (I), (II) or (III). Examples of pharmaceutically acceptable solvates include, but are not limited to water, isopropanol, ethanol, methanol, DMSO, ethyl acetate, acetic acid, and ethanolamine.

By “ring system substituent” is meant a substituent attached to an aromatic or non-aromatic ring system. When a ring system is saturated or partially saturated the “ring system substituent” further includes methylene (double bonded carbon), oxo (double bonded oxygen) or thioxo (double bonded sulfur).

The term “spiroalkyl” as used herein, represents an alkylene diradical, both ends of which are bonded to the same carbon atom of the parent group to form a spirocyclic group.

The term “sulfonyl” as used herein, represents an S(O)₂ group.

The term “thioalkoxy” as used herein, represents an alkyl group attached to the parent molecular group through a sulfur atom. Exemplary unsubstituted thioalkoxy groups are of from 1 to 6 carbons.

The term “thioalkoxyalkyl” is represents a thioalkoxy group attached to the parent molecular group through an alkyl group.

By the terms “thiocarbonyl” or “thiooxo” is meant a C(S) group, which can also be represented as C═S.

By the terms “thiol” or “sulfhydryl” is meant an SH group.

Compounds that have the same molecular formula but differ in the nature or sequence of bonding of their atoms or the arrangement of their atoms in space are termed “isomers”. Isomers in which the connectivity between atoms is the same but which differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn, Ingold and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

Asymmetric or chiral centers may exist in the compounds of the present invention. Unless indicated otherwise, the description or naming of a particular compound in the specification and claims is intended to include all individual enantiomers and mixtures, racemic or otherwise, thereof. The methods for the determination of stereochemistry and the separation of stereoisomers are well known in the art (see discussion in Chapter 4 of “Advanced Organic Chemistry”, 4th edition J. March, John Wiley and Sons, New York, 1992). Individual stereoisomers of compounds or the present invention are prepared synthetically from commercially available starting materials that contain asymmetric or chiral centers or by preparation of mixtures of enantiomeric compounds followed by resolution well-known to those of ordinary skill in the art. These methods of resolution are exemplified by (1) attachment of a racemic mixture of enantiomers, designated (±), to a chiral auxiliary, separation of the resulting diastereomers by recrystallization or chromatography and liberation of the optically pure product from the auxiliary or (2) direct separation of the mixture of optical enantiomers on chiral chromatographic columns. Enantiomers are designated herein by the symbols “R” or “S,” depending on the configuration of substituents around the chiral carbon atom, or are drawn by conventional means with a bolded line defining a substituent above the plane of the page in three-dimensional space and a hashed or dashed line defining a substituent beneath the plane of the printed page in three-dimensional space.

As generally understood by those skilled in the art, an optically pure compound is one that is enantiomerically pure. As used herein, the term “optically pure” is intended to mean a compound that comprises at least a sufficient amount of a single enantiomer to yield a compound having the desired pharmacological activity. Preferably, “optically pure” is intended to mean a compound that comprises at least 90% of a single isomer (80% enantiomeric excess, i.e., “e.e.”), preferably at least 95% (90% e.e.), more preferably at least 97.5% (95% e.e.), and most preferably at least 99% (98% e.e.). Preferably, the compounds of the invention are optically pure.

B) Compounds according to the invention

As will be described in details hereinafter, the inventors have prepared series of analogs of 4-hydroxyisoleucine. According to preferred embodiments of the invention, these analogs are potentially active for stimulating glucose uptake and/or stimulating insulin secretion in mammals, and can therefore be useful for preventing and/or treating disorders in which elevated glucose levels are problematic. Consequently, providing such analogs is not only desirable for the treatment of diabetes, but also for the treatment of other disorders of carbohydrate metabolism.

According to a first aspect, the present invention features analogs of 4-hydroxyisoleucine, such as those having Formula (I): 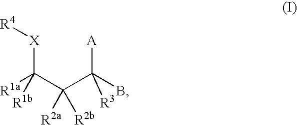
and pharmaceutically acceptable lactones, salts, prodrugs, metabolites or solvates thereof.

The substituent A in a compound of Formula (I) can be CO₂R^{A1}, C(O)SR^{A1}, C(S)SR^{A1}, C(O)NR^{A2}R^{A3}, C(S)NR^{A2}R^{A3}, C(O)R^{A4}, SO₃H, S(O)₂NR^{A2}R^{A3}, C(O)R^{A5}, C(OR^{A1})R^{A9}R^{A10}, C(SR^{A1})R^{A9}R^{A10}, C(═NR^{A1})R^{A5},

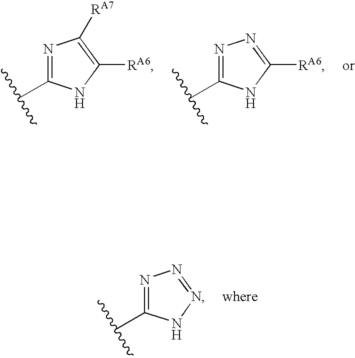

R^{A1} is hydrogen, substituted or unsubstituted C₁₋₆ alkyl, substituted or unsubstituted C₃₋₈ cycloalkyl, substituted or unsubstituted alkcycloalkyl, where the cycloalkyl group is of three to eight carbon atoms and the alkylene group is of one to four carbon atoms, substituted or unsubstituted C₂₋₆ alkenyl, substituted or unsubstituted C₂₋₆ alkynyl, substituted or unsubstituted C₆ or C₁₀ aryl, substituted or unsubstituted C₇₋₁₆ alkaryl, where the alkylene group is of one to four carbon atoms, substituted or unsubstituted C₁₋₉ heterocyclyl, or substituted or unsubstituted C₂₋₁₅ alkheterocyclyl, where the alkylene group is of one to four carbon atoms,

each of R^{A2} and R^{A3} is, independently, selected from the group consisting of (a) hydrogen, (b) substituted or unsubstituted C₁₋₆ alkyl, (c) substituted or unsubstituted C₃₋₈ cycloalkyl, (d) substituted or unsubstituted alkcycloalkyl, where the cycloalkyl group is of three to eight carbon atoms and the alkylene group is of one to four carbon atoms, (e) substituted or unsubstituted C₆ or C¹⁰ aryl, and (D substituted or unsubstituted C₇₋₁₆ alkaryl, where the alkylene group is of one to six carbon atoms, or R^{A2} taken together with R^{A3} and N forms a substituted or unsubsituted 5- or 6-membered ring, optionally containing O or NR^{A8}, where R^{A8} is hydrogen or C₁₋₆ alkyl,

R^{A4} is substituted or unsubstituted C₁₋₆ alkyl, substituted or unsubstituted C₃₋₈ cycloalkyl, substituted or unsubstituted alkcycloalkyl, where the cycloalkyl group is of three to eight carbon atoms and the alkylene group is of one to four carbon atoms, substituted or unsubstituted C₆ or C₁₀ aryl, substituted or unsubstituted C₇₋₁₆ alkaryl, where the alkylene group is of one to four carbon atoms, substituted or unsubstituted C₁₋₉ heterocyclyl, or substituted or unsubstituted C₂₋₁₅ alkheterocyclyl, where the alkylene group is of one to four carbon atoms,

R^{A5} is a peptide chain of 1-4 natural or unnatural amino acids, where the peptide is linked via its terminal amine group to C(O),

each of R^{A6} and R^{A7} is, independently, hydrogen, substituted or unsubstituted C₁₋₆ alkyl, C₁₋₄ perfluoroalkyl, substituted or unsubstituted C₁₋₆ alkoxy, amino, C₁₋₆ alkylamino, C₂₋₁₂ dialkylamino, N-protected amino, halo, or nitro, and

each of R^{A9} and R^{A10} is, independently, selected from the group consisting of (a) hydrogen, (b) substituted or unsubstituted C₁₋₆ alkyl, (c) substituted or unsubstituted C₃₋₈ cycloalkyl, (d) substituted or unsubstituted alkcycloalkyl, where the cycloalkyl group is of three to eight carbon atoms and the alkylene group is of one to four carbon atoms, (e) substituted or unsubstituted C₆ or C₁₀ aryl, and (f) substituted or unsubstituted C₇₋₁₆ alkaryl, where the alkylene group is of one to six carbon atoms, or R^{A9} taken together with R^{A10} and their parent carbon atom forms a substituted or unsubsituted 5- or 6-membered ring, optionally containing O or NR^{A8}, wherein R^{A8} is hydrogen or C₁₋₆ alkyl.

The substituent B in a compound of Formula (I) can be NR^{B1}R^{B2}, where each of R^{B1} and R^{B2} is, independently selected from the group consisting of (a) hydrogen, (b) an N-protecting group, (c) substituted or unsubstituted C₁₋₆ alkyl, (d) substituted or unsubstituted C₂₋₆ alkenyl, (e) substituted or unsubstituted C₂₋₆ alkynyl, (f) substituted or unsubstituted C₃₋₈ cycloalkyl, (g) substituted or unsubstituted alkcycloalkyl, where the cycloalkyl group is of three to eight carbon atoms, and the alkylene group is of one to ten carbon atoms, (h) substituted or unsubstituted C₆ or C₁₀ aryl, (i) substituted or unsubstituted C₇₋₁₆ alkaryl, where the alkylene group is of one to six carbon atoms, (j) substituted or unsubstituted C₁₋₉ heterocyclyl, (k) substituted or unsubstituted C₂₋₁₅ alkheterocyclyl, where the alkylene group is of one to six carbon atoms, (I) C(O)R^{B3}, where R^{B3} is selected from the group consisting of substituted or unsubstituted C₁₋₆ alkyl, substituted or unsubstituted C₆ or C₁₀ aryl, substituted or unsubstituted C₇₋₁₆ alkaryl, where the alkylene group is of one to six carbon atoms, substituted or unsubstituted C₁₋₉ heterocyclyl, or substituted or unsubstituted C₂₋₁₅ alkheterocyclyl, where the alkylene group is of one to six carbon atoms, (m) CO₂R^{B4}, where R^{B4} is selected from the group consisting of substituted or unsubstituted C₁₋₆ alkyl, substituted or unsubstituted C₆ or C₁₀ aryl, substituted or unsubstituted C₇₋₁₆ alkaryl, where the alkylene group is of one to six carbon atoms, substituted or unsubstituted C₁₋₉ heterocyclyl, or substituted or unsubstituted C₂₋₁₅ alkheterocyclyl, where the alkylene group is of one to six carbon atoms, (n) C(O)NR^{B5}R^{B6}, where each of R^{B5} and R^{B6} is, independently, selected from the group consisting of hydrogen, substituted or unsubstituted C₁₋₆ alkyl, substituted or unsubstituted C₆ or C₁₀ aryl, substituted or unsubstituted C₇₋₁₆ alkaryl, where the alkylene group is of one to six carbon atoms, substituted or unsubstituted C₁₋₉ heterocyclyl, and substituted or unsubstituted C₂₋₁₅ alkheterocyclyl, where the alkylene group is of one to six carbon atoms, or R^{B5} taken together with R^{B6} and N forms a substituted or unsubsituted 5- or 6-membered ring, optionally containing a non-vicinal O, S, or NR′, where R′ is H or C₁₋₆ alkyl, (o) S(O)₂R^{B7}, where R^{B7} is selected from the group consisting of substituted or unsubstituted C₁₋₆ alkyl, substituted or unsubstituted C₆ or C₁₀ aryl, substituted or unsubstituted C₇₋₁₆ alkaryl, where the alkylene group is of one to six carbon atoms, substituted or unsubstituted C₁₋₉ heterocyclyl, or substituted or unsubstituted C₂₋₁₅ alkheterocyclyl, where the alkylene group is of one to six carbon atoms, and (p) a peptide chain of 1-4 natural or unnatural alpha-amino acid residues, where the peptide is linked via its terminal carboxy group to N, with the proviso that no two groups are bound to the nitrogen atom through a carbonyl group or a sulfonyl group. Alternatively, R^{B1} can form ring systems when combined with other substituents of Formula I. In one ring system, R^{B1} taken together with R^{B2} and N forms a substituted or unsubstituted 5- or 6-membered ring, optionally containing O or NR^{B8}, wherein R^{B8} is hydrogen or C₁₋₆ alkyl. Alternatively, a 5- to 8-membered ring is formed when R^{B1} taken together with R^{1a} is a substituted or unsubstituted C₁₋₄ alkyl or a [2.2.1] or [2.2.2] bicyclic ring system is formed when R^{B1} taken together with R^{1a} is a substituted or unsubstituted C₂ alkylene and R^{B1} taken together with R^{2a} is a substituted or unsubstituted C₁₋₂ alkylene. Alternatively, a 4- to 8-membered ring is formed when R^{B1} taken together with R³ is a substituted or unsubstituted C₂₋₆ alkyl. A 6- to 8-membered ring can be formed when R^{B1} taken together with R⁴ is a substituted or unsubstituted C₁₋₃ alkyl. Yet another ring is formed when R^{B1} taken together with A and the parent carbon of A and B form the following ring: 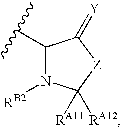
where each of Y and Z is, independently, O, S, NR^{B8}, or CR^{A9}R^{A10}, where each of R^{A9} and R^{A10} is as previously defined and each of R^{A11} and R^{A12} is, independently, selected from the group consisting of (a) hydrogen, (b) substituted or unsubstituted C₁₋₆ alkyl, (c) substituted or unsubstituted C₃₋₈ cycloalkyl, (d) substituted or unsubstituted alkcycloalkyl, where the cycloalkyl group is of three to eight carbon atoms and the alkylene group is of one to four carbon atoms, (e) substituted or unsubstituted C₆ or C₁₀ aryl, and (f) substituted or unsubstituted C₇₋₁₆ alkaryl, where the alkylene group is of one to six carbon atoms, or R^{A9} taken together with R^{A10} and their parent carbon atom forms a substituted or unsubsituted 5- or 6-membered ring, optionally containing O or NR^{A8}, wherein R^{A3} is hydrogen or C₁₋₆ alkyl. In one embodiment, the B′ substituent does not form rings with R^{1a}, R^{1b} or R⁴.

The substituent X in a compound of Formula (I) can be O, S, or NR^{X1}, where R^{X1} is selected from the group consisting of (a) hydrogen, (b) an N-protecting group, (c) substituted or unsubstituted C₁₋₆ alkyl, (d) substituted or unsubstituted C₂₋₆ alkenyl, (e) substituted or unsubstituted C₂₋₆ alkynyl, (f substituted or unsubstituted C₃₋₈ cycloalkyl, (g) substituted or unsubstituted alkcycloalkyl, where the cycloalkyl group is of three to eight carbon atoms, and the alkylene group is of one to ten carbon atoms, (h) substituted or unsubstituted C₆ or C₁₀ aryl, (i) substituted or unsubstituted C₇₋₁₆ alkaryl, where the alkylene group is of one to six carbon atoms, (j) substituted or unsubstituted C₁₋₉ heterocyclyl, or (k) substituted or unsubstituted C₂₋₁₅ alkheterocyclyl, where the alkylene group is of one to six carbon atoms.

For a compound of Formula (I), each of the R^{1a} and R^{1b} substituents is, independently, substituted or unsubstituted C₁₋₆ alkyl, substituted or unsubstituted C₃₋₈ cycloalkyl, substituted or unsubstituted alkcycloalkyl, where the cycloalkyl group is of three to eight carbon atoms and the alkylene group is of one to four carbon atoms, substituted or unsubstituted C₂₋₆ alkenyl, substituted or unsubstituted C₂₋₆ alkynyl, substituted or unsubstituted C₆ or C₁₀ aryl, substituted or unsubstituted C₇₋₁₆ alkaryl, where the alkylene group is of one to four carbon atoms, substituted or unsubstituted C₁₋₉ heterocyclyl, or substituted or unsubstituted C₂₋₁₅ alkheterocyclyl, where the alkylene group is of one to four carbon atoms, or R^{1a} together with R^{2a} and their base carbon atoms form a substituted or unsubstituted C₅₋₁₀ mono or fused ring system, or a 3- to 6-membered ring is formed when R^{1a} together with R⁴ is a substituted or unsubstituted C₁₋₄ alkylene.

For a compound of Formula (I), each of the R^{2a} and R^{2b} is, independently, hydrogen, substituted or unsubstituted C₁₋₆ alkyl, substituted or unsubstituted C₃₋₈ cycloalkyl, substituted or unsubstituted alkcycloalkyl, where the cycloalkyl group is of three to eight carbon atoms and the alkylene group is of one to four carbon atoms, substituted or unsubstituted C₂₋₆ alkenyl, substituted or unsubstituted C₂₋₆ alkynyl, substituted or unsubstituted C₆ or C₁₀ aryl, substituted or unsubstituted C₇₋₁₆ alkaryl, where the alkylene group is of one to four carbon atoms, substituted or unsubstituted C₁₋₉ heterocyclyl, or substituted or unsubstituted C₂₋₁₅ alkheterocyclyl, where the alkylene group is of one to four carbon atoms, or R^{2a} and R^{2b} together are ═O, ═N(C₁₋₆ alkyl), ═CR²CR^{2d}, where each of R^{2c} and R^{2d} is, independently, hydrogen or substituted or unsubstituted C₁₋₆ alkyl, or a substituted or unsubstitued C₂₋₅ alkylene moiety forming a spiro ring, or R^{2a} together with R^{1a} and their base carbon atoms form a substituted or unsubstituted C₅₋₁₀ mono or fused ring system.

The substituent R³ in a compound of Formula (I) can be hydrogen, substituted or unsubstituted C₁₋₆ alkyl, substituted or unsubstituted alkcycloalkyl, where the cycloalkyl group is of three to eight carbon atoms and the alkylene group is of one to four carbon atoms, substituted or unsubstituted C₂₋₆ alkenyl, substituted or unsubstituted C₂₋₆ alkynyl, substituted or unsubstituted C₇₋₁₆ alkaryl, where the alkylene group is of one to four carbon atoms, or substituted or unsubstituted C₂₋₁₅ alkheterocyclyl, where the alkylene group is of one to four carbon atoms. Alternatively, a 4- to 8-membered ring can be formed when R³ taken together with R^{B1} is a substituted or unsubstituted C₂₋₆ alkylene.

The substituent R⁴ in a compound of Formula (I) is hydrogen, substituted or unsubstituted C₁₋₆ alkyl, substituted or unsubstituted C₃₋₈ cycloalkyl, substituted or unsubstituted alkcycloalkyl, where the cycloalkyl group is of three to eight carbon atoms and the alkylene group is of one to four carbon atoms, substituted or unsubstituted C₂₋₆ alkenyl, substituted or unsubstituted C₂₋₆ alkynyl, substituted or unsubstituted C₆ or C₁₀ aryl, substituted or unsubstituted C₇₋₁₆ alkaryl, where the alkylene group is of one to four carbon atoms, substituted or unsubstituted C₁₋₉ heterocyclyl, or substituted or unsubstituted C₂₋₁₅ alkheterocyclyl, where the alkylene group is of one to four carbon atoms, or a 3- to 6-membered ring is formed when R⁴ together with R^{1a} is a substituted or unsubstituted C₁₋₄ alkylene, or a 6- to 8-membered ring is formed when R⁴ taken together with R^{B1} is a substituted or unsubstituted C₁₋₃ alkylene.

In certain embodiments, the analogs of the present invention are represented by generalized Formula (I) and the attendant definitions, wherein A is CO₂H, B⁴ is NH-p-toluenesulfonyl, R⁴ is H and each of R^{1a} and R^{2a} is CH₃.

In certain embodiments, the analogs of the present invention are represented by generalized Formula (I) and the attendant definitions, wherein A is CO₂H, B is NH₂, R⁴ is H and each of R^{1a} and R^{2a} is a substituted or unsubstituted C₁₋₆ alkyl.

In certain embodiments, the analogs of the present invention are represented by generalized Formula (I) and the attendant definitions, wherein R^{1a} together with R^{2a} and their base carbon atoms form a substituted or unsubstituted C₅₋₁₀ mono or fused ring system, optionally containing a non-vicinal O, S, or NR′, where R′ is H or C₁₋₆ alkyl.

In certain embodiments, the analogs of the present invention are represented by generalized Formula (II), or a pharmaceutically acceptable lactone, salt, metabolite, solvate and/or prodrug thereof:

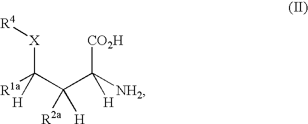

where each of R^{1a} and R^{2a} is, independently, substituted or unsubstituted C₁₋₆ alkyl or R^{1a} together with R^{2a} and their base carbon atoms form a substituted or unsubstituted C₆ alicyclic ring system. In certain embodiments, the analogs of the present invention are represented by generalized Formula (II) and the attendant definitions, wherein R^{1a} represents an ethyl group, R^{2a} represents a methyl group, X represents O and R4 represents an hydrogen atom. Some examples of this embodiment include compounds identified as having ID Nos 13b, 12b, 218, 219, 220, 221, 222, and 223 in Table 1 hereinafter.

In certain embodiments, the analogs of the present invention are represented by generalized Formula (II) and the attendant definitions, wherein X represents O, R⁴ represents an hydrogen atom, and R^{1a} and R^{2a} join to form a six or seven membered ring structure. Some examples of this embodiment include compounds identified as having ID Nos 12e, 13e, 14e, 15e, 213, 214, 215, 216, 217, 12f, 13f, 14f, 15f, 231, 232, 233, 234, and 235 in Table 1 hereinafter.

In certain embodiments, the analogs of the present invention are represented by generalized Formula (II) and the attendant definitions, wherein R^{1a} represents a methyl group, R^{2a} represents a benzyl group, X represents O and R⁴ represents an hydrogen atom. Some examples of this embodiment include compounds identified as having ID Nos 12d, 13d, 14d, 15d, 238, 239, 240, and 241 in Table 1 hereinafter.

Yet, in some embodiments, the analogs of the present invention are represented by generalized Formula (I) and the attendant definitions, wherein R^{1a}, R^{1b} and R^{2a} represent methyl groups, X represents O and R⁴ represents a hydrogen atom. Some examples of this embodiment include compounds identified as having ID Nos 207, 101a, 101b, 208, 209, 210 in Table 1 hereinafter. Desirable compounds of this embodiment have the 2S,3R configuration.

In certain embodiments, the analogs of the present invention are represented by generalized Formula (III), or a pharmaceutically acceptable lactone, salt, metabolite, solvate and/or prodrug thereof: 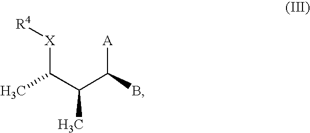
where each of B, X, and R⁴ is as defined elsewhere herein and A is CO₂R^{A1}, C(O)SR^{A1}, C(O)NR^{A2}R^{A3}, or C(O)R^{A5}.

In certain embodiments, the analogs of the present invention are represented by generalized Formula (IV), or a pharmaceutically acceptable lactone, salt, metabolite, solvate and/or prodrug thereof: 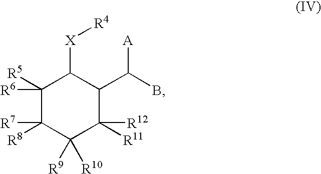
where each of B, X, and R⁴ is as defined elsewhere herein, A is CO₂R^{A1}, C(O)SR^{A1}, C(O)NR^{A2}R^{A3}, or C(O)R^{A5}, and R⁵, R⁶, R⁷, R⁸, R⁹, R¹⁰, R¹¹, and R¹² are, independently, hydrogen, substituted or unsubstituted C₁₋₆ alkyl, substituted or unsubstituted C₃₋₈ cycloalkyl, substituted or unsubstituted alkcycloalkyl, where the cycloalkyl group is of three to eight carbon atoms and the alkylene group is of one to four carbon atoms, substituted or unsubstituted C₂₋₆ alkenyl, substituted or unsubstituted C₂₋₆ alkynyl, substituted or unsubstituted C₆ or C₁₀ aryl, substituted or unsubstituted C₇₋₁₆ alkaryl, where the alkylene group is of one to four carbon atoms, substituted or unsubstituted C₁₋₉ heterocyclyl, or substituted or unsubstituted C₂₋₁₅ alkheterocyclyl, where the alkylene group is of one to four carbon atoms Desirable compounds of this embodiment have the SSR-configuration.

In certain embodiments, the compounds of the present invention are represented by generalized Formulae, or a pharmaceutically acceptable lactone, salt, and/or prodrug thereof: 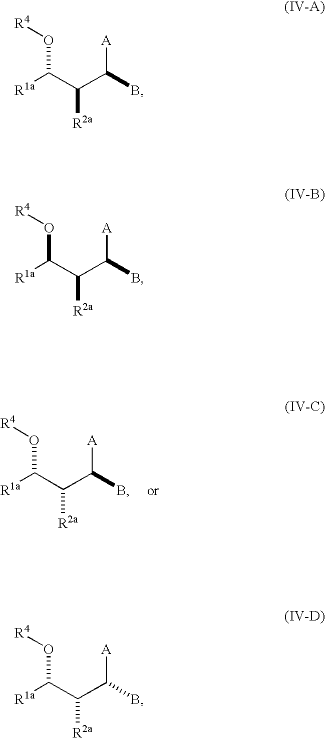
where each of R^{1a} and R^{2a} is, individually, substituted or unsubstituted C₁₋₆ alkyl, substituted or unsubstituted C₃₋₈ cycloalkyl, substituted or unsubstituted alkcycloalkyl, where the cycloalkyl group is of three to eight carbon atoms and the alkylene group is of one to four carbon atoms, substituted or unsubstituted C₂₋₆ alkenyl, substituted or unsubstituted C₂₋₆ alkynyl, substituted or unsubstituted C₆ or C₁₀ aryl, substituted or unsubstituted C₇₋₁₆ alkaryl, where the alkylene group is of one to four carbon atoms, substituted or unsubstituted C₁₋₉ heterocyclyl, or substituted or unsubstituted C₂₋₁₅ alkheterocyclyl, where the alkylene group is of one to four carbon atoms.

In one preferred example of this embodiment, A is CO₂H, B is NH₂, R⁴ is H, and each of R^{1a} and R^{2a} is a substituted or unsubstituted C₁₋₆ alkyl. In another example, preferable analogs of 4-OH include those compounds where R^{1a} together with R^{2a} and their base carbon atoms form a substituted or unsubstituted C₅₋₁₀ mono or fused ring system, such as, for example, a compound selected from the group consisting of: 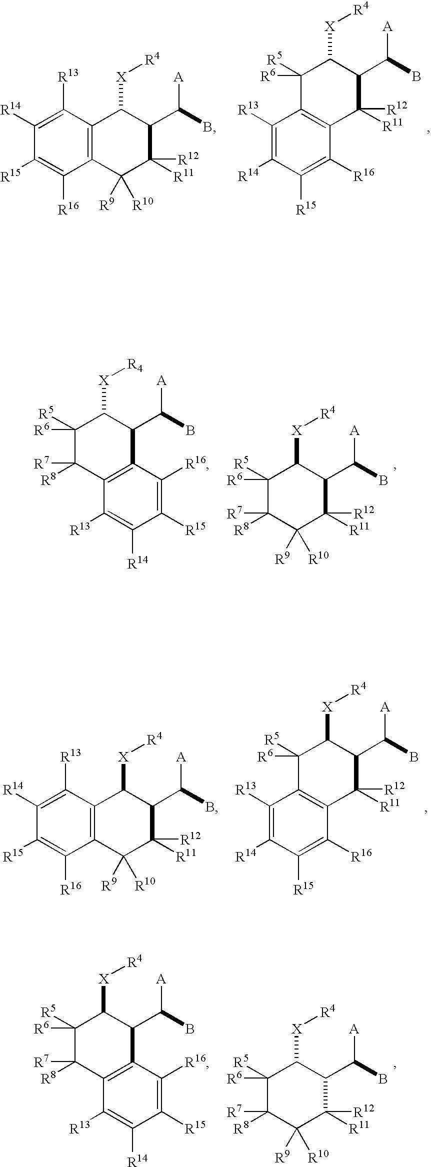 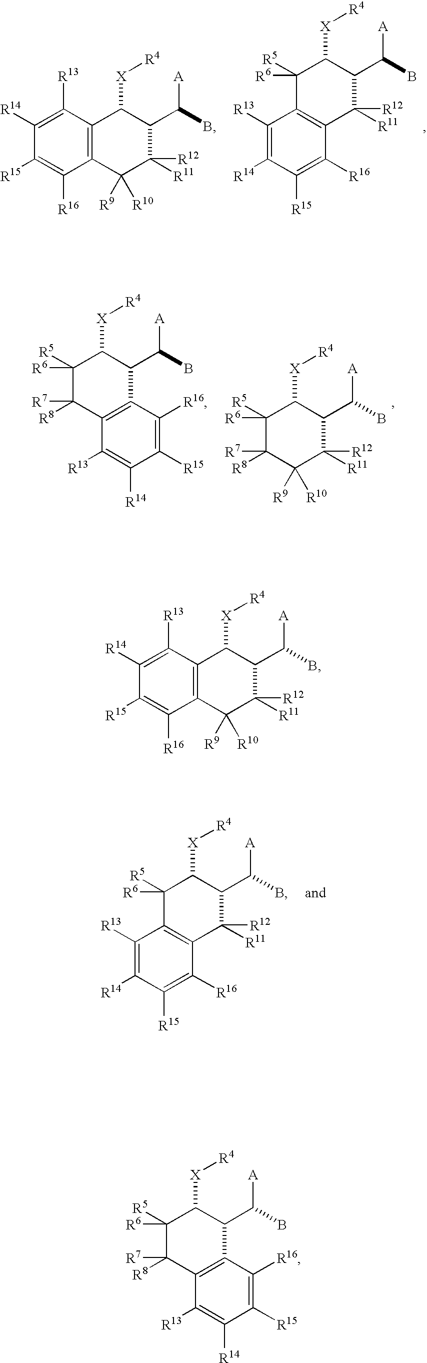
where each of R⁵, R⁶, R⁷, R⁸, R⁹, R¹⁰, R¹¹, and R¹² is, independently, hydrogen, substituted or unsubstituted C₁₋₆ alkyl, substituted or unsubstituted C₃₋₈ cycloalkyl, substituted or unsubstituted alkcycloalkyl, where the cycloalkyl group is of three to eight carbon atoms and the alkylene group is of one to four carbon atoms, substituted or unsubstituted C₂₋₆ alkenyl, substituted or unsubstituted C₂₋₆ alkynyl, substituted or unsubstituted C₆ or C₁₀ aryl, substituted or unsubstituted C₇₋₁₆ alkaryl, where the alkylene group is of one to four carbon atoms, substituted or unsubstituted C₁₋₉ heterocyclyl, or substituted or unsubstituted C₂₋₁₅ alkheterocyclyl, where the alkylene group is of one to four carbon atoms; and each of R¹³, R¹⁴, R¹⁵, and R¹⁶ is, independently, hydrogen, substituted or unsubstituted C₁₋₆ alkyl, C₁₋₄ perfluoroalkyl, substituted or unsubstituted C₁₋₆ alkoxy, amino, C₁₋₆ alkylamino, C₂₋₁₂ dialkylamino, N-protected amino, halo, or nitro. Most preferable compounds in this series are those in which A is CO₂H and B is NH₂.

In another embodiment, the compound of Formula (I) is 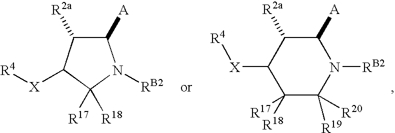
where each of R¹⁷, R¹⁸, R¹⁹, and R²⁰ is hydrogen or substituted or unsubstituted C₁₋₆ alkyl.

In another embodiment, the compound of Formula (I) is 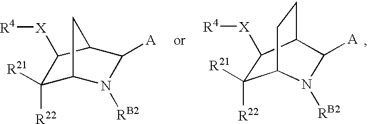
where each of R²¹ and R²² is hydrogen or substituted or unsubstituted C₁₋₆ alkyl.

In yet another embodiment, the compound of Formula (I) is

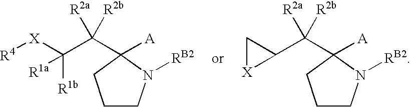

Other examples of a compound of Formula (I) include a compound selected from the group of compounds identified as having ID Nos 22, 26, 33, 34, 75, 76, 205, 206, 65, 59, 60, 61, 62, 200, 201, 202, 38, 99, 99a, 99b, 100, 100a, 100b, 207, 101a, 101b, 12c, 13c, 14c, 226, 230, 253 and 254 in Table I hereinafter.

Additional examples of a compound of Formula (I) include compounds selected from the group of compounds identified as having ID Nos 204, 102a, 102b, 211, 5a, 82, 203, 5c, 7c, and 225 in Table 1 hereinafter.

According to some embodiment, the invention excludes compounds of Formula (I) that are configurational isomers of 4-hydroxyisoleucine or configurational isomers of 4-hydroxyisoleucine γ-lactone. According to other embodiments, the invention exclude compounds of Formula (I) that are are configurational isomers of: 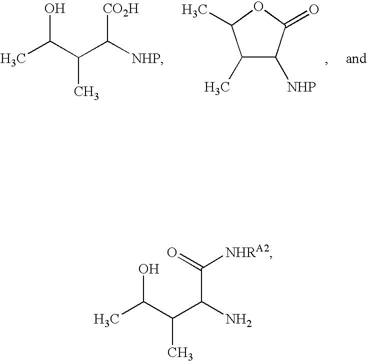
wherein P is hydrogen or a nitrogen protecting group and R^{A2} is as previously defined.

The invention also encompasses salts, solvates, crystal forms, active metabolites and prodrugs of the compounds of Formulae (I), (II), and (III). Specific examples of prodrugs include, but is not limited to compounds of Formulae (I), (II), or (III) wherein a suitable functionality, such as, but not exclusively, a hydroxy, amino, or sulfhydryl group in Formulae (I), (II), and/or (III) is properly derivatized with a biologically or chemically labile molecular moiety that may be cleaved in vivo to regenerate a compound of Formulae (I), (II), or (III).

In other embodiments, the analogs of the invention are selected from the group consisting of the compounds listed hereinafter in Table 1. It should be noted that in Table 1 hereinafter and throughout the present document when an atom is shown without hydrogen(s), but hydrogens are required or chemically necessary to form a stable compound, hydrogens should be inferred to be part of the compound.

TABLE 1
Structures of Exemplary Compounds

| Cpd # | Structure |
|---|---|
| 5a | 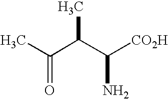 |
| 5b | 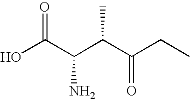 |
| 5c | 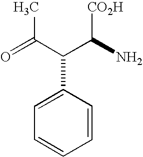 |
| 5d | 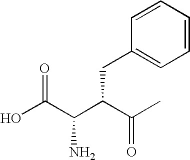 |
| 5e | 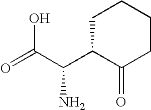 |
| 5f | 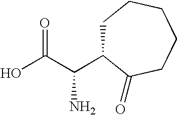 |
| 7b | 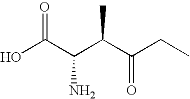 |
| 7c | 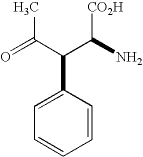 |
| 7d | 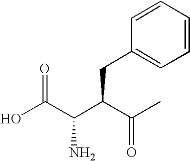 |
| 7e | 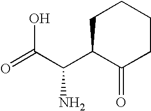 |
| 7f | 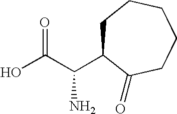 |
| 12b | 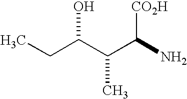 |
| 12c | 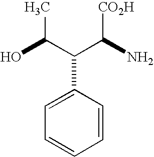 |
| 12d | 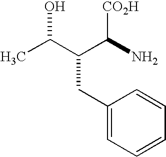 |
| 12e | 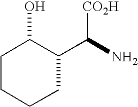 |
| 12f | 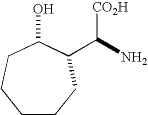 |
| 13b | 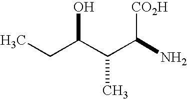 |
| 13c | 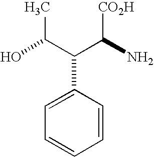 |
| 13d | 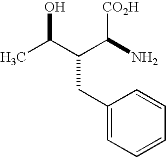 |
| 13e | 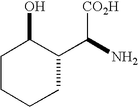 |
| 13f | 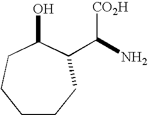 |
| 14a | 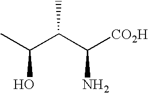 |
| 14c | 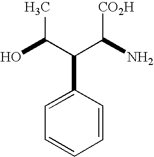 |
| 14d | 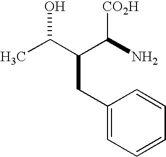 |
| 14e | 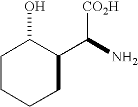 |
| 14f | 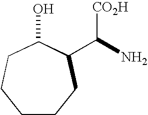 |
| 15b | 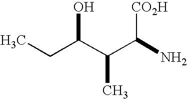 |
| 15c | 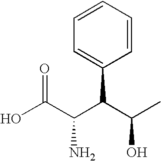 |
| 15d | 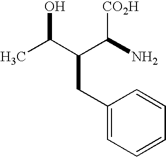 |
| 15e | 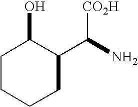 |
| 15f | 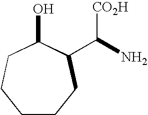 |
| 22 | 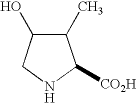 |
| 26 | 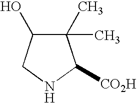 |
| 33 | 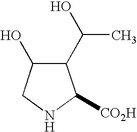 |
| 34 | 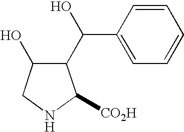 |
| 38 | 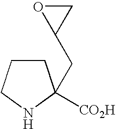 |
| 40 | 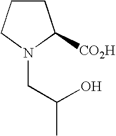 |
| 59 | 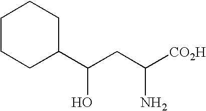 |
| 60 | 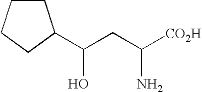 |
| 61 | 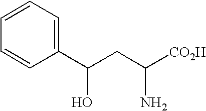 |
| 62 | 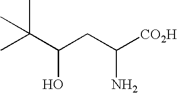 |
| 65 | 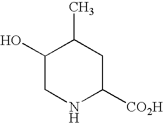 |
| 67 | 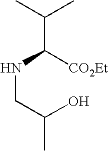 |
| 75 | 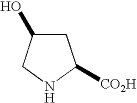 |
| 76 | 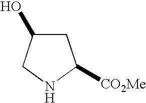 |
| 77 | 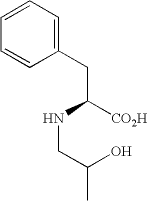 |
| 82 | 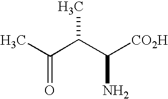 |
| 99 | 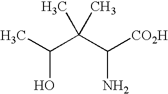 |
| 99a | 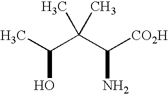 |
| 99b | 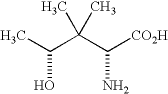 |
| 100 | 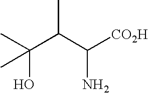 |
| 100a | 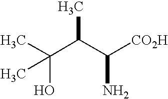 |
| 100b | 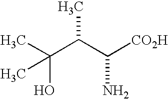 |
| 101a | 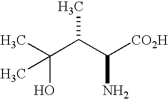 |
| 101b | 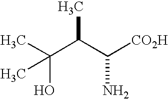 |
| 102a | 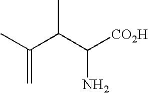 |
| 102b | 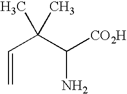 |
| 104 | 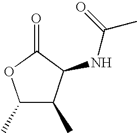 |
| 105 | 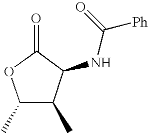 |
| 107a | 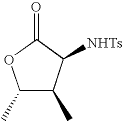 |
| 107b | 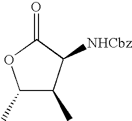 |
| 108a | 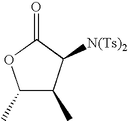 |
| 108b | 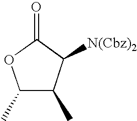 |
| 109 | 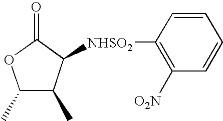 |
| 110 | 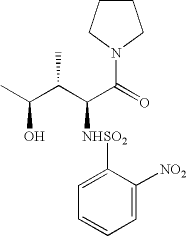 |
| 111a | 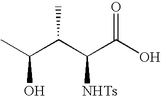 |
| 111b | 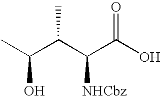 |
| 112a | 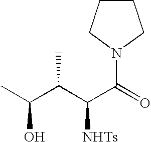 |
| 112b | 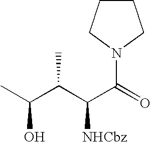 |
| 113a | 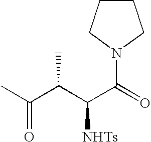 |
| 113b | 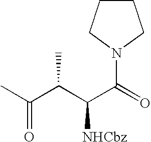 |
| 116 | 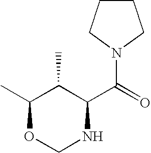 |
| 117 | 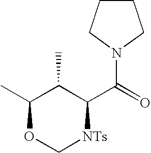 |
| 118 | 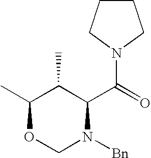 |
| 119 | 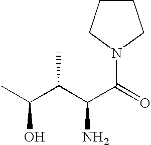 |
| 120 | 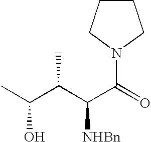 |
| 121a | 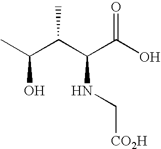 |
| 121b | 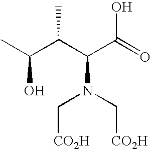 |
| 122 | 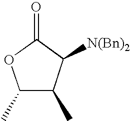 |
| 123 | 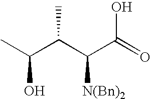 |
| 128 | 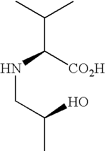 |
| 133 | 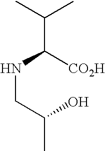 |
| 200 | 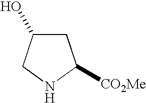 |
| 201 | 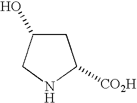 |
| 202 | 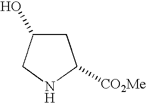 |
| 203 | 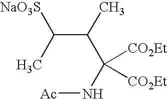 |
| 204 | 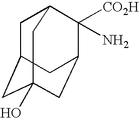 |
| 205 | 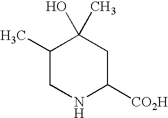 |
| 206 | 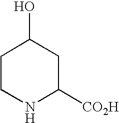 |
| 207 | 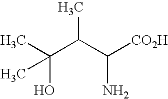 |
| 208 | 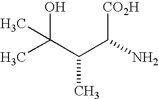 |
| 209 | 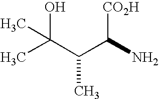 |
| 210 | 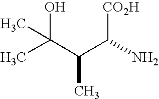 |
| 211 | 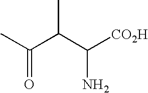 |
| 212 | 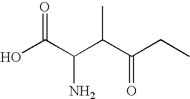 |
| 213 | 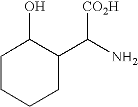 |
| 214 | 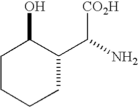 |
| 215 | 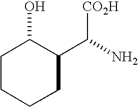 |
| 216 | 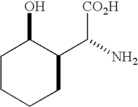 |
| 217 | 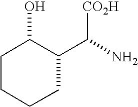 |
| 218 | 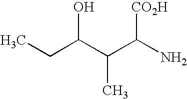 |
| 219 | 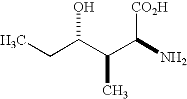 |
| 220 | 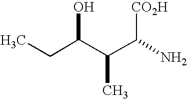 |
| 221 | 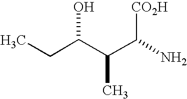 |
| 222 | 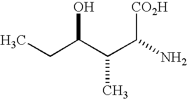 |
| 223 | 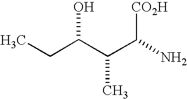 |
| 224 | 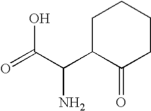 |
| 225 | 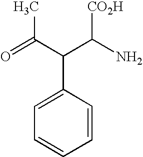 |
| 226 | 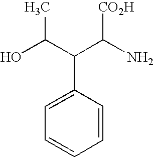 |
| 229 | 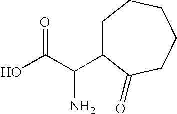 |
| 230 | 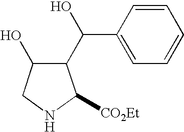 |
| 231 | 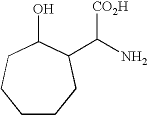 |
| 232 | 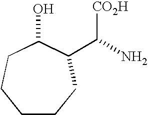 |
| 233 | 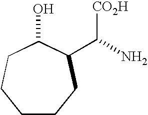 |
| 234 | 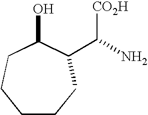 |
| 235 | 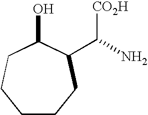 |
| 236 | 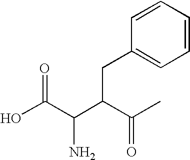 |
| 238 | 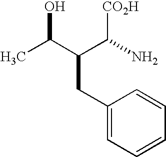 |
| 239 | 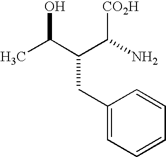 |
| 240 | 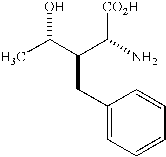 |
| 241 | 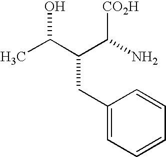 |
| 242 | 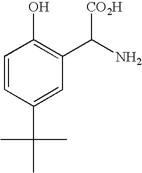 |
| 243 | 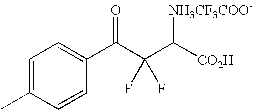 |
| 244 | 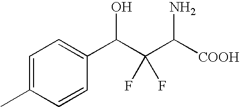 |
| 245 | 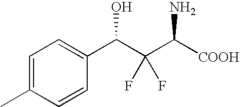 |
| 246 | 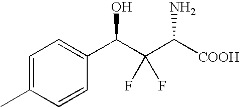 |
| 247 | 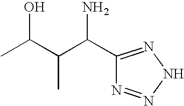 |
| 248 | 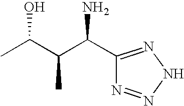 |
| 249 | 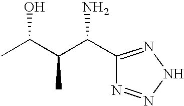 |
| 250 | 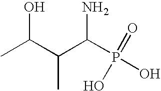 |
| 251 | 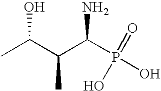 |
| 252 | 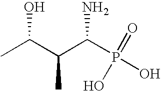 |
| 253 | 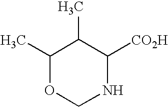 |
| 254 | 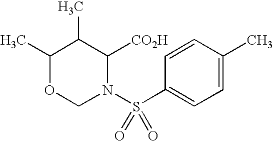 |
| 255 | 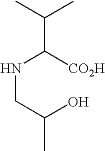 |

The compounds and compositions (see hereinafter) of the invention may be prepared by employing the techniques available in the art using starting materials that are readily available. For instance, methods for the preparation of (2S,3R,4S)-4-hydroxyisoleucine have been described, see for example U.S. Patent Application Publication No. US 2003/0219880; Rolland-Fulcrand et al., Eur. J. Org. Chem. 873-877, 2004; and Wang et al., Eur. J. Org. Chem. 834-839, 2002. In addition, this compound can be isolated from the seeds of fenugreek (Trigonella foenum-graecum). Methods for making additional configurational isomers of 4-hydroxyisoleucine, or prodrug thereof, have also been described in PCT/FR2005/02805 filed Nov. 10, 2005 (WO 2006/______ published on May ______, 2006) which is incorporated herein by reference.

An additional aspect of the invention concerns new methods for the synthesis of analogs according to the invention. Certain novel and exemplary methods of preparing the inventive compounds are described in the Exemplification section. Such methods are within the scope of this invention.

C) Methods for Stimulating Glucose Uptake and Methods for Stimulating Insulin Secretion.

The compounds of the invention preferably stimulate glucose uptake by muscle tissues or adipose tissues and/or stimulate insulin secretion by pancreatic β-cells. The biological activity of the compounds of the invention may be measured by any of the methods available to those skilled in the art, including in vivo and in vitro assays. Some examples of suitable assays for such measurement are described herein in the Exemplification section. Additional examples of suitable art-recognized assays for such measurement are well known.

Accordingly, a related aspect, the invention provides a method of stimulating glucose uptake by muscle and or adipose tissues, the method comprising:
providing at least one analog according to the invention as defined herein;
providing a functional in vitro cell-based assay in which glucose uptake stimulation is assessable; and
introducing an effective amount of said analog(s) into the assay for stimulating glucose uptake activity.

In one embodiment, the in vitro cell-based assay comprises 3T3-L1 adipocytes cells and is carried out in presence of about 10 μM 2-Deoxy-D-glucose and about 16 μM ³H-Deoxy-D-glucose.

Accordingly, a related aspect, the invention provides a method of stimulating insulin secretion by β-cells, the method comprising:
providing at least one analog according to the invention as defined herein;
providing a functional in vitro cell-based assay in which stimulation of insulin secretion is assessable; and
introducing an effective amount of said analog(s) into the assay for stimulating insulin secretion.

In one embodiment, the in vitro cell-based assay comprises INS-1 cells and is carried out in presence of a glucose concentration of about 2 mM to about 10 mM.

D) Pharmaceutical Compositions and Therapeutic Applications

Without wishing to be bound by theory, the inventors have demonstrated that the analogs of the invention are suitable for stimulating glucose uptake, and/or stimulating insulin secretion. Therefore, present invention pertains to methods of using the analogs of 4-OH and pharmaceutical compositions thereof for treatment or prevention purposes. In preferred embodiments, the method compromises administering any of the individual compounds described herein, or any combination thereof.

According to preferred embodiments of the invention, the mammal is a human subject in need of treatment by the methods and/or analogs of the invention, and is selected for treatment based on this need. A human in need of treatment, especially when referring to type 2 diabetes is art-recognized and includes subjects that have been identified as having abnormally high blood glucose levels, a reduced glucose tolerance, a disregulation of fat metabolisms, and may have a surplus of weight (e.g. obese). Humans in need of treatment may also be at risk of such a disease or disorder, and would be expected based on diagnosis, e.g., medical diagnosis, to benefit from treatment (e.g., curing, healing, preventing, alleviating, relieving, altering, remedying, ameliorating, improving, or affecting the disease or disorder, the symptom of the disease or disorder, or the risk of the disease or disorder).

Therefore, a related aspect of the invention concerns the use of analogs of the invention as an active ingredient in a pharmaceutical composition for treatment or prevention purposes. As used herein, “treating” or “treatment” is intended to mean at least the mitigation of a disease condition associated with a disorder of carbohydrate or lipid metabolism, and more particularly type 2 diabetes in a mammal, such as a human, that is alleviated by a stimulation of insulin secretion and/or by a stimulation of glucose uptake, and includes curing, healing, inhibiting (e.g. arresting or reducing the development of the disease or its clinical symptoms), relieving from, improving and/or alleviating, in whole or in part, the disease condition (e.g. causing regression of the disease or its clinical symptoms).

As used herein, “prophylaxis” or “prevent” or “prevention” is intended to mean at least the reduction of likelihood of a disease condition associated with a disorder of carbohydrate or lipid metabolism, and more particularly type 2 diabetes in humans. Type 2 diabetes predisposing factors identified or proposed in the scientific literature include, among others, (i) a genetic predisposition to having the disease condition but not yet diagnosed as having it, (ii) being obese, (iii) having a disregulation of fat metabolism and/or (iv) having a sedentary life style. For example, it is likely that one can prevent or treat type 2 diabetes in a human by administering an analog of the invention or a composition comprising the same, when the human is at a pre-diabetic state, when the human is overweight, when the human shows abnormally high blood glucose levels, and/or when the human exhibits a reduced tolerance to glucose.

The subject may be a female human or a male human, and it may be a kid, a teenage or an adult.

According to a specific aspect, the invention features a method for treating a mammal, such as a human, having diabetes mellitus (type 1 or type 2 diabetes), pre-diabetes, or Metabolic Syndrome, that includes administering to the mammal an analog of the invention, and/or a composition comprising the same, in an amount sufficient to decrease its circulating glucose level.

According to certain embodiments, the analogs, compositions and methods of the invention are administered at a therapeutically effective dosage sufficient to reduce the glucose levels in a subject's plasma, from about at least 5, 10, 15, 20 25, 30, 40, 50, 75 or 100 percent, when compared to original levels prior to treatment.

According to certain embodiments, the analogs, compositions and methods of the invention are administered at a therapeutically effective dosage sufficient to increase insulin levels in a subject's plasma from about at least 5, 10, 15, 20 25, 30, 40, 50, 75 or 100 percent, when compared to original levels prior to treatment.

Typically, the analogs of the invention are given until glucose and/or insulin levels go back to normal. Due to the nature of the disorders and conditions targeted by the analogs of the invention, it is likely that a chronic or lifetime administration is going to be required. In preferred embodiments, analogs and pharmaceutical composition according to the invention are administered once to thrice a day.

The amount of glucose or insulin in the blood, or plasma of a subject can be evaluated by using techniques and methods well known to those skilled in the art, including but not limited to hand-held glucometer, enzymatic assays (e.g. glucose oxidase or hexokinase bases assays) enzyme-linked immunosorbent assay (“ELISA”), quantitative immunoblotting test methods, and radiolabeled immunoassay (RIA).

Therefore, the present invention provides pharmaceutical compositions comprising a therapeutically effective amount of an analog of 4-OH as described herein in combination with a pharmaceutically acceptable carrier or excipient. Suitable carriers or excipients include, but are not limited to saline, buffered saline, dextrose, water, glycerol, ethanol, and combinations thereof. The pharmaceutical compositions may be administered in any effective, convenient manner including, for instance, administration by topical, parenteral, oral, anal, intravaginal, intravenous, intraperitoneal, intramuscular, intraocular, subcutaneous, intranasal, intrabronchial, or intradermal routes among others.

Acceptable methods of preparing suitable pharmaceutical forms of the pharmaceutical compositions are known to those skilled in the art. For example, pharmaceutical preparations may be prepared following conventional techniques of the pharmaceutical chemist involving steps such as mixing, granulating, and compressing when necessary for tablet forms, or mixing, filling, and dissolving the ingredients as appropriate, to give the desired products for various routes of administration.

Toxicity and therapeutic efficacy of the analogs according to the invention can be evaluated by standard pharmaceutical procedures in cell cultures or experimental animals. The therapeutic efficacy of the analogs according to the invention can be evaluated in an animal model system that may be predictive of efficacy in human diseases. For instance, animal models for evaluating efficacy in glucose uptake include animal models for diabetes or other relevant animal models in which glucose infusion rate can be measured. Animal model for evaluating insulinotropic efficacy include animal models for diabetes or other relevant animal models in which secretion of insulin can be measured. Examples of suitable animal models for diabetes include, but are not limited to DIO mice, ob/ob mice, db/db mice, and Zucker fa/fa rats. Alternatively, the ability of an analog can be evaluated in vitro, by examining the ability of the compound to stimulate glucose uptake using differentiated 3T3-L1 adipocyte cells (see Example 2) or using L6 myocytes, by examining the ability of the compound to stimulate insulin secretion using INS-1 cells (see Example 3) or using perfused pancreas. While agents that exhibit toxic side effects may be used, care should be taken to design a delivery system that targets such agents to the site of affected tissue in order to minimize potential damage to unaffected cells and, thereby, reduce side effects.

A wide range of drugs can be used with the analogs, compositions and methods of the present invention. Such drugs may be selected from antidiabetic agents, antihypertensive agents, anti-inflammatory agents, antiobesity agents, etc.

A non-limitative list of useful antidiabetic agents that can be used in combination with an analog of the invention include insulin, biguanides, such as, for example metformin (Glucophage®, Bristol-Myers Squibb Company, U.S.; Stagid®, Lipha Santé, Europe); sulfonylurea drugs, such as, for example, gliclazide (Diamicron®), glibenclamide, glipizide (Glucotrol® and Glucotrol XL®, Pfizer), glimepiride (Amaryl®, Aventis), chlorpropamide (e.g., Diabinese®, Pfizer), tolbutamide, and glyburide (e.g., Micronase®, Glynase®, and Diabeta®); glinides, such as, for example, repaglinide (Prandin® or NovoNorm®; Novo Nordisk), ormitiglinide, nateglinide (Starlix®), senaglinide, and BTS-67582; insulin sensitizing agents, such as, for example, glitazones, a thiazolidinedione such as rosiglitazone maleate (Avandia®, Glaxo Smith Kline), pioglitazone (Actos®, Eli Lilly, Takeda), troglitazone, ciglitazone, isaglitazone, darglitazone, englitazone, CS-011/CI-1037, T 174, GI 262570, YM-440, MCC-555, JTT-501, AR-H039242, KRP-297, GW-409544, CRE-16336, AR-H049020, LY510929, MBX-102, CLX-0940, GW-501516, and the compounds described in WO 97/41097 (DRF-2344), WO 97/41119, WO 97/41120, WO 98/45292, WO 99/19313 (NN622/DRF-2725), WO 00/23415, WO 00/23416, WO 00/23417, WO 00/23425, WO 00/23445, WO 00/23451, WO 00/41121, WO 00/50414, WO 00/63153, WO 00/63189, WO 00/63190, WO 00/63191, WO 00/63192, WO 00/63193, WO 00/63196, and WO 00/63209; glucagon-like peptide 1 (GLP-1) receptor agonists, such as, for example, Exendin-4 (1-39) (Ex-4), Byetta™ (Amylin Pharmaceuticals Inc.), CJC-1131 (Conjuchem Inc.), NN-2211 (Scios Inc.), and those GLP-1 agonists described in WO 98/08871 and WO 00/42026; agents that slow down carbohydrate absorption, such as, for example, α-glucosidase inhibitors (e.g., acarbose, miglitol, voglibose, and emiglitate); agents that inhibit gastric emptying, such as, for example, glucagon-like peptide 1, cholescystokinin, amylin, and pramlintide; glucagon antagonists, such as, for example, quinoxaline derivatives (e.g., 2-styryl-3-[3-(dimethylamino)propylmethylamino]-6,7-dichloroquinoxaline, Collins et al., Bioorganic and Medicinal Chemistry Letters 2(9):915-918, 1992), skyrin and skyrin analogs (e.g., those described in WO 94/14426), 1-phenyl pyrazole derivatives (e.g., those described in U.S. Pat. No. 4,359,474), substituted disilacyclohexanes (e.g., those described in U.S. Pat. No. 4,374,130), substituted pyridines and biphenyls (e.g., those described in WO 98/04528), substituted pyridyl pyrroles (e.g., those described in U.S. Pat. No. 5,776,954), 2,4-diaryl-5-pyridylimidazoles (e.g., those described in WO 98/21957, WO 98/22108, WO 98/22109, and U.S. Pat. No. 5,880,139), 2,5-substituted aryl pyrroles (e.g., those described in WO 97/16442 and U.S. Pat. No. 5,837,719), substituted pyrimidinone, pyridone, and pyrimidine compounds (e.g., those described in WO 98/24780, WO 98/24782, WO 99/24404, and WO 99/32448), 2-(benzimidazol-2-ylthio)-1-(3,4-dihydroxyphenyl)-1-ethanones (see Madsen et al., J. Med. Chem. 41:5151-5157, 1998), alkylidene hydrazides (e.g., those described in WO 99/01423 and WO 00/39088), and other compounds, such as those described in WO 00/69810, WO 02/00612, WO 02/40444, WO 02/40445, and WO 02/40446; and glucokinase activators, such as, for example, those described in WO 00/58293, WO 01/44216, WO 01/83465, WO 01/83478, WO 01/85706, and WO 01/85707.

Other examples of antidiabetic agents that can be used in combination with one or more analogs according to the invention include imidazolines (e.g., efaroxan, idazoxan, phentolamine, and 1-phenyl-2-(imidazolin-2-yl)benzimidazole); glycogen phosphorylase inhibitors (see, e.g., WO 97/09040); oxadiazolidinediones, dipeptidyl peptidase-IV (DPP-IV) inhibitors, protein tyrosine phosphatase (PTPase) inhibitors, inhibitors of hepatic enzymes involved in stimulation of gluconeogenesis and/or glycogenolysis, glucose uptake modulators, glycogen synthase kinase-3 (GSK-3) inhibitors, compounds that modify lipid metabolism (e.g., antihyperlipidemic agents and antilipidemic agents), peroxisome proliferator-activated receptor (PPAR) agonists or antagonists in general, retinoid X receptor (RXR) agonists (e.g., ALRT-268, LG-1268, and LG-1069), and antihyperlipidemic agents or antilipidemic agents (e.g., cholestyramine, colestipol, clofibrate, gemfibrozil, lovastatin, pravastatin, simvastatin, probucol, and dextrothyroxine). Other suitable antidiabetic agents are listed in Table 2, provided elsewhere herein.

Examples of antihypertensive agents that can be used with the analogs of the invention include β-blockers (e.g., alprenolol, atenolol, timolol, pindolol, propranolol, and metoprolol), angiotensin converting enzyme (ACE) inhibitors (e.g., benazepril, captopril, enalapril, fosinopril, lisinopril, quinapril, and ramipril), calcium channel blockers (e.g., nifedipine, felodipine, nicardipine, isradipine, nimodipine, diltiazem, and verapamil), and α-blockers (e.g., doxazosin, urapidil, prazosin, and terazosin).

Examples of anti-inflammatory agents that can be used with the analogs of the invention include anti-histamines, and anti-TNFα.

Examples of anti-obesity agents that can be used with the analogs of the invention include Xenical™ (Roche), Meridia™ (Abbott) Acomplia™ (Sanofi-Aventis), Pramlintide (Amylin) and sympathomimetic phentermine.

The isomers, compositions and methods of the present invention may also be used with isomers of 4-OH, such as those decribed in the PCT application untitled “DIASTEREOISOMERS OF 4-HYDROXYISOLEUCINE AND USES THEREOF” which claims priority of US Provisional Application 60/654,413 filed Feb. 18, 2005.

Accordingly, another aspect of relates to a pharmaceutical kit or pharmaceutical composition that includes any of the analogs of 4-OH described herein, or any combination thereof, and a second antidiabetic agent. The pharmaceutical kit or composition can include a 4-hydroxyisoleucine analog and a second antidiabetic agent that is formulated into a single composition, such as, for example, a tablet or a capsule. The invention also provides methods of treating diabetes (type 1 diabetes or type 2 diabetes), pre-diabetes, or Metabolic Syndrome in patients, which include administering to a patient one or more analogs of 4-hydroxyisoleucine such as those described herein, in combination with one or more antidiabetic agents. The combination of agents can be administered at or about the same time as one another or at different times.

The combinations of the invention provide several advantages. For example, because the drug combinations described herein can be used to obtain an improved (e.g., additive or synergistic) effect, it is possible to consider administering less of each drug, leading to a decrease in the overall exposure of patients to drugs, as well as any untoward side effects of any of the drugs. In addition, greater control of the disease may be achieved, because the drugs can combat the disease through different mechanisms.

Administration

With respect to the therapeutic methods of the invention, it is not intended that the administration of compounds to a mammal be limited to a particular mode of administration, dosage, or frequency of dosing; the present invention includes all modes of administration, including oral, intraperitoneal, intramuscular, intravenous, intra-articular, intralesional, subcutaneous, by inhalation, or any other route sufficient to provide a dose adequate to prevent or treat diabetes (type 1 diabetes or type 2 diabetes) and other disorders of carbohydrate or lipid metabolism, such as those described herein. One or more compounds may be administered to the mammal in a single dose or multiple doses. When multiple doses are administered, the doses may be separated from one another by, for example, several hours, one day, or one week. It is to be understood that, for any particular subject, specific dosage regimes should be adjusted over time according to the individual need and the professional judgment of the person administering or supervising the administration of the compositions. Exemplary mammals that can be treated using the analogs, compositions and methods of the invention include humans, primates such as monkeys, animals of veterinary interest (e.g., cows, pigs, sheep, goats, buffaloes, and horses) and domestic pets (e.g., dogs and cats). The analogs and compositions of the invention could also be administered to rodents (e.g. mice, rats, gerbils, hamsters, guinea pigs, and rabbits) for treatment purposes and/or for experimental purposes (e.g. studying the compounds' mechanism(s) of action, screening and testing efficacy of the analogs, structural design, etc.)

For clinical applications in therapy or as a prophylactic, analogs or compositions of the present invention may generally be administered, e.g., orally, subcutaneously, parenterally, intravenously, intramuscularly, colonically, nasally, intraperitoneally, rectally, by inhalation, or buccally. Compositions containing at least one analog of 4-hydroxyisoleucine according to the invention that is suitable for use in human or veterinary medicine may be presented in forms permitting administration by a suitable route. These compositions may be prepared according to customary methods, using one or more pharmaceutically acceptable carriers or excipients. The carriers comprise, among other things, diluents, sterile aqueous media, and various non-toxic organic solvents. Acceptable carriers or diluents for therapeutic use are well known in the pharmaceutical field, and are described, for example, in Remington: The Science and Practice of Pharmacy (20th ed.), ed. A. R. Gennaro, Lippincott Williams & Wilkins, 2000, Philadelphia, and Encyclopedia of Pharmaceutical Technology, eds. J. Swarbrick and J. C. Boylan, 1988-1999, Marcel Dekker, New York. The compositions may be presented in the form of tablets, pills, granules, powders, aqueous solutions or suspensions, injectable solutions, elixirs, or syrups, and the compositions may optionally contain one or more agents chosen from the group comprising sweeteners, flavorings, colorings, and stabilizers in order to obtain pharmaceutically acceptable preparations.

The choice of vehicle and the content of active substance in the vehicle are generally determined in accordance with the solubility and chemical properties of the product, the particular mode of administration, and the provisions to be observed in pharmaceutical practice. For example, excipients such as sodium citrate, calcium carbonate, and dicalcium phosphate and disintegrating agents such as starch, alginic acids, and certain complex silicates combined with lubricants (e.g., magnesium stearate, sodium lauryl sulfate, and talc) may be used for preparing tablets. To prepare a capsule, it is advantageous to use high molecular weight polyethylene glycols. When aqueous suspensions are used, they may contain emulsifying agents that facilitate suspension. Diluents such as ethanol, polyethylene glycol, propylene glycol, glycerol, chloroform, or mixtures thereof may also be used. In addition, low calorie sweeteners, such as, for example, isomalt, sorbitol, xylitol, may be used in a formulation of the invention.

For parenteral administration, emulsions, suspensions, or solutions of the compositions of the invention in vegetable oil (e.g., sesame oil, groundnut oil, or olive oil), aqueous-organic solutions (e.g., water and propylene glycol), injectable organic esters (e.g., ethyl oleate), or sterile aqueous solutions of the pharmaceutically acceptable salts can be used. The solutions of the salts of the compositions of the invention are especially useful for administration by intramuscular or subcutaneous injection. Aqueous solutions that include solutions of the salts in pure distilled water may be used for intravenous administration with the proviso that (i) their pH is adjusted suitably, (ii) they are appropriately buffered and rendered isotonic with a sufficient quantity of sodium chloride, and (iii) they are sterilized by heating, irradiation, or microfiltration. Suitable compositions containing the analogs of the invention may be dissolved or suspended in a suitable carrier for use in a nebulizer or a suspension or solution aerosol, or may be absorbed or adsorbed onto a suitable solid carrier for use in a dry powder inhaler. Solid compositions for rectal administration include suppositories formulated in accordance with known methods. It is understood that the appropriate doses and concentrations of the agent(s) in the formulations (i.e. analog(s) of 4-hydroxyisoleucine alone and/or in combination with other drug(s)) will vary, depending on a number of factors including the dosages of the agents to be administered, the route of administration, the nature of the agent(s), the frequency and mode of administration, the therapy desired, the form in which the agent(s) are administered, the potency of the agent(s), the sex, age, weight, and general condition of the subject to be treated, the nature and severity of the condition treated, any concomitant diseases to be treated, and other factors that will be apparent to those of skill in the art. A dose of the pharmaceutical composition contains at least a therapeutically effective amount of an analog according to the invention and is preferably made up of one or more pharmaceutical dosage units. The selected dose may be administered to a human subject in need of treatment. A “therapeutically effective amount” is intended to mean that amount of analog(s) of the invention that confers a therapeutic effect on the subject treated. The therapeutic effect may be objective (i.e. measurable by some test or marker (e.g., insulin or glucose levels) or subjective (i.e. the subject gives an indication of or feels an effect).

A dose of the pharmaceutical composition contains at least a therapeutically effective amount of an analog according to the invention and is preferably made up of one or more pharmaceutical dosage units. The selected dose may be administered to a mammal, for example, a human patient, in need of treatment. A “therapeutically effective amount” is intended to mean that amount of analog(s) according to the invention that, when administered to a subject for treating a disease, confers a therapeutic effect on the subject treated. The therapeutic effect may be objective (i.e. measurable by some test or marker (e.g. insulin or glucose blood levels) or subjective (i.e. the subject gives an indication of or feels an effect). For instance, in one embodiment relating to type 2 diabetes, a “therapeutically effective” amount will increase glucose uptake by muscle and/or adipose tissues, and/or it will stimulate insulin secretion by pancreatic β-cells. In another embodiment relating to type 2 diabetes, a “therapeutically effective” amount reduces glucose levels and/or increase insulin levels in the subject's blood by, for example, at least about 20%, or by at least about 40%, or even by at least about 60%, or by at least about 80% relative to untreated subjects.

The amount that will correspond to a “therapeutically effective amount” will vary depending upon factors such as the particular compound, the route of administration, excipient usage, the disease condition and the severity thereof, the identity of the subject in need thereof, the age, weight, etc., of the subject to be treated and the possibility of co-usage with other agents for treating a disease. Nevertheless the therapeutically effective amount can be readily determined by one of skill in the art.

For administration to mammals, and particularly humans, it is expected that in the treatment of an adult dosages from about 0.1 mg to about 50 mg (e.g., about 5 mg to about 100 mg, about 1 mg to about 50 mg, or about 5 mg to about 25 mg) of each active compound per kg body weight per day can be used. A typical oral dosage can be, for example, in the range of from about 50 mg to about 5 g per day (e.g., about 100 mg to about 4 g, 250 mg to 3 g, or 500 mg to 2 g), administered in one or more dosages, such as 1 to 3 dosages. Dosages can be increased or decreased as needed, as can readily be determined by those of skill in the art. For example, the amount of a particular agent can be decreased when used in combination with another agent, if determined to be appropriate. In addition, reference can be made to standard amounts and approaches that are used to administer the agents mentioned herein.

Examples of dosages for antidiabetic agents mentioned herein are provided in Table 2, below. The antidiabetic agents can be used in these dosages when combined with an analog of 4-hydroxyisoleucine, which generally is administered in an amount in the range of, for example, 250 mg-1 g/day (e.g., 350-900, 450-800, or 550-700 mg/day). Alternatively, due to the potential additive or synergistic effects obtained when using drug combinations of the invention, the amounts in Table 2 and/or the amount of hydroxylated amino acid administered can be decreased (by, e.g., about 10-70%, 20-60%, 30-50%, or 35-45%), as determined to be appropriate by those of skill in this art.

The physician in any event will determine the actual dosage that will be most suitable for an individual. The above dosages are exemplary of the average case. There can, of course, be individual instances where higher or lower dosage ranges are merited, and such are within the scope of this invention.

As for dosing, it is understood that duration of a treatment using any of the compounds or compositions of the invention will vary depending on several factors, such as those listed herein before for dosing. Nevertheless, appropriate duration of administration can be readily determined by one of skill in the art. According to certain embodiments, the compounds of the invention are administered on a daily, weekly or on a continuous basis.

TABLE 2
List of well-known antidiabetic agents

| | Recommended dosage |
|---|---|
| Antidiabetic agent | and/or administration |
| Insulin | 400 IU per vial - 40 IU per day |
| | (mean value) |
| Gliclazide (Diamicron) | 80 mg/tablet - 1 to 4 tablets per day |
| Glibenclamide (Daonil) | 5 mg/tablet - 1 to 3 tablets per day |
| or Glyburide (Micronase, | (Glibenclamide); 1.25 to 6 mg/tablet - |
| Glynase, Diabeta) | 1 to 2 tablets per day (Glyburide) |
| Glipizide (Glucotrol, | 5 mg/tablet - 1 to 4 tablets per day |
| Glibenese) | |
| Glimepiride (Amaryl, | 1 to 4 mg/tablet - 6 mg per day maximum |
| Amarel) | |
| Chlorpropamide | 250 mg/tablet - 125 to 1000 mg |
| (Diabinese) | per day per day |
| Tolbutamide | 500 mg/tablet - 1 to 4 tablets per day |
| Repaglinide (Prandin) | 0.5 to 16 mg per day |
| Nateglinide, | 60 to 120 mg/tablet - 3 tablets per day |
| Senaglinide | |
| (Starlix) | |
| Tolazamide | 100 to 500 mg/tablet |
| Rosiglitazone | 2 to 8 mg/tablet - 8 mg per day maximum |
| Pioglitazone | 15 to 45 mg/tablet - 15 to 45 mg per day |
| Troglitazone | 200 to 400 mg/tablet - 200 to 600 mg |
| | per day |
| Ciglitazone | 0.1 mg/tablet |
| Exetanide (Amylin) | 0.09 to 0.270 mg per day |
| Acarbose | 50 to 100 mg/tablet - 150 to 600 mg |
| | per day |
| Miglitol | 50 to 100 mg/tablet - 150 to 300 mg |
| | per day |
| Voglibose | 0.1 to 0.9 mg per day |
| Phentolamine | 50 mg - 4 to 6 times per day |
| Cholestyramine | 4 g/unit - 12 to 16 g per day |
| (Colestipol) | |
| Clofibrate | 500 mg/capsule - 1 to 4 capsules per day |
| Gemfibrozil (Lipur) | 450 mg/tablet - 2 tablets per day |
| Lovastatin | 10 and 20 mg/tablet |
| Pravastatin | 20 mg/tablet - 10 to 40 mg per day |
| Simvastatin (Zocor, | 5 and 20 mg/tablet - 5 to 40 mg per day |
| Lodales) | |
| Probucol | 250 mg/tablet - 1 g per day |
| Dextrothyroxine | 2 to 6 mg per day |
| Alprenolol | 50 mg/tablet - 4 to 8 tablets per day |
| Atenolol | 50 to 100 mg/tablet - 100 to 200 mg |
| | per day |
| Timolol | 10 mg/tablet - 10 to 20 mg per day |
| Pindolol | 5 and 15 mg/tablet - 5 to 60 mg per day |
| Propranolol | 40 mg/tablet - 80 to 160 mg per day |
| Metoprolol | 100 and 200 mg/tablet - 50 to 200 mg |
| | per day |
| Captopril | 25 and 50 mg/tablet - 12.5 to 150 mg |
| | per day |
| Enalapril | 5 and 20 mg/tablet - 5 to 40 mg per day |
| Nifedipine | 10 mg/capsule - 30 to 60 mg per day |
| Diltiazem | 60 mg/tablet - 3 to 6 tablets per day |
| Verapamil | 120 and 240 mg/capsule - 240 to 360 mg |
| | per day |
| Doxazosin | 2 to 8 mg per day |
| Prazozin | 2.5 and 5 mg/tablet - 2.5 to 20 mg |
| | per day |

The analogs and compositions of the invention are conceived to be effective primarily in the treatment of disorders of carbohydrate metabolism, particularly type 2 diabetes. However, it is conceivable that the analogs and compositions according to the present invention may also be useful in connection with disorders of fat metabolism, including but not limited to lipodystrophy associated with HIV and lipidemia, because they may influence fat distribution.

It is also conceivable to use analogs of the invention for others related or unrelated applications. For instance, it might be useful to provide in-dwelling devices such as catheters coated with the compounds of the invention, for improving cardiovascular functions.

### EXAMPLES

The Examples set forth herein below provide exemplary syntheses of certain representative compounds of the invention. Also provided are exemplary methods for assaying the compounds of the invention for their activity as stimulators of glucose uptake and as stimulators of insulin secretion. These examples are given to enable those skilled in the art to more closely understand and to practice the present invention and are not intended to either define or limit its scope.

### Example 1

### General Procedure for the Preparation of Analogs of 4-hydroxyisoleucine

A) General Experimental Procedures

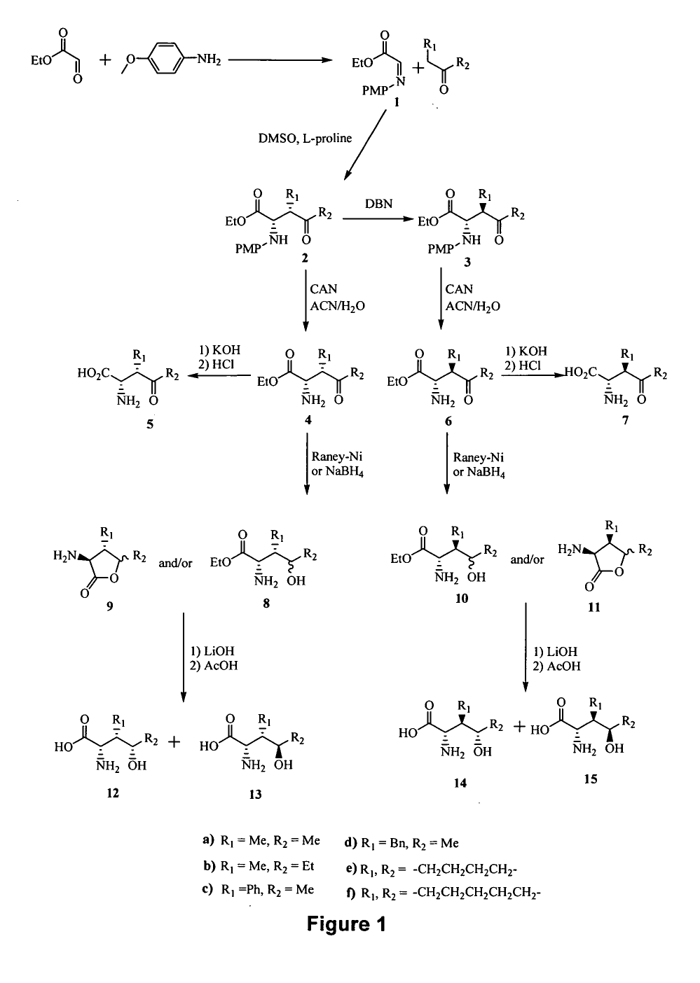

Reference is made to FIGS. 1 to 14 showing synthetic schemes for the synthesis of exemplary linear and cyclic analogs of 4-hydroxyisoleucine.

FIG. 1 shows synthesis of various analogs of 4-hydroxyisoleucine with SSS, SSR, SRS and SRR configuration. Imine intermediate I was prepared from p-anisidine and ethyl glyoxalate (Cordova et al., J. Am. Chem. Soc. 124:1842-43, 2002). The reaction of imine 1 with a suitable ketone in the presence of L-Proline as a catalyst yielded 2S,3S isomer (2). Epimerization at C-3 was achieved with a base, e.g., 1,5-diazabicyclo[4.3.0]non-5-ene (DBN) to yield 2S,3R isomer (3). The (2S,3S,4S), (2S,3S,4R), (2S,3R,4S) and (2S,3R,4R) analogs of 4-hydroxyisoleucine were obtained from 2 or 3, respectively, as follows:

Deprotection of amine moiety of 2 (removal of p-methoxyphenyl group) with ceric ammonium nitrate (CAN) to yield 4 and subsequent hydrolysis led to (2S,3S)-4-keto analogs (5). Similarly, deprotection of 3 yielded 6 which upon base hydrolysis gave (2S,3R)-4-keto analogs (7). The reduction of 4 and 6 with NaBH₄ or Raney nickel or as a single step deprotection/reduction of 2 and 3 generated a diastereomeric mixture of a lactone (9 & 11) and an open chain intermediate (8 & 10), respectively. The hydrolysis of a mixture of 8 and 9, followed by purification gave (2S,3S,4S) and (2S,3S,4R) analogs, 12 and 13, respectively. Similarly, (2S,3R,4S) and (2S,3R,4R) analogs, i.e., 14 and 15, were obtained form the hydrolysis of a mixture of compounds 10 and 11.

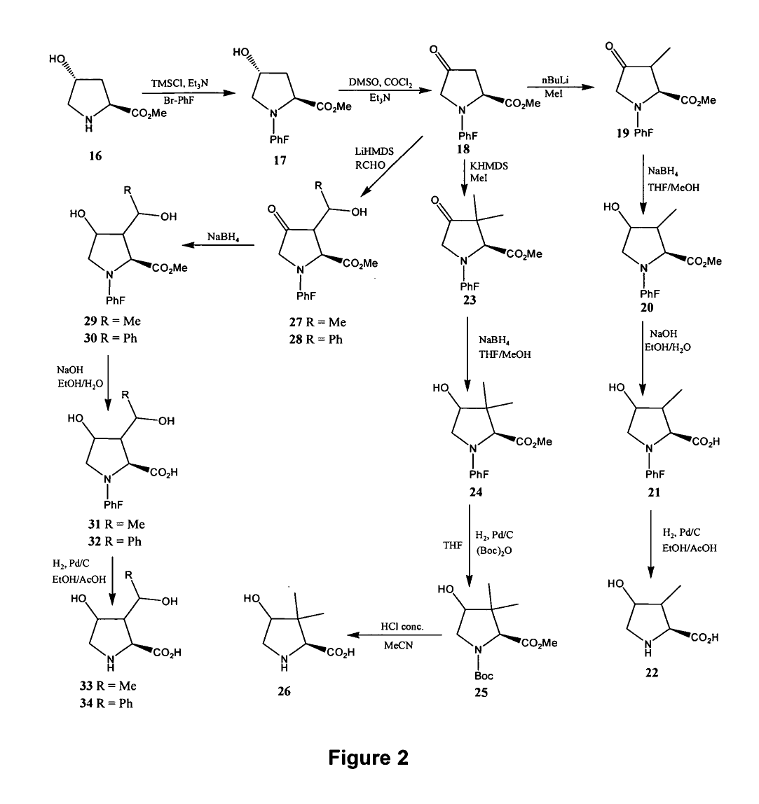

3-substitued 4-hydroxyproline based analogs were synthesized as depicted in FIG. 2. 4-Hydroxyproline methyl ester (16) reaction with chlorotrimethylsilane, triethylamine, followed by reaction with bromo-phenylfluorene/Pb(NO₃)₂ gave the protected intermediate (17). Swern oxidation of 17 with oxalylchloride and DMSO led to the key intermediate PhF-4-oxoproline methyl ester (18). Alkylation at C-3 of this intermediate gave various 3-substituted analogs. Mono-alkylation of 18 was achieved using n-Buthyllithium as a base to give compound 19, while di-alkylation was performed using KHMDS as a base gave compound 23. The reduction of alkylated oxoproline intermediates (19 & 23) gave the hydroxyl intermediates, 20 and 24, respectively. The base hydrolysis of 20 gave the acid (21), which upon catalytic hydrogenolysis affored the desired 3-methyl analog (22). The corresponding dimethyl intermediate (24) underwent catalytic hydrogenolysis and in-situ protection with boc anhydride to yield the Boc intermediate (25), which upon deprotection and acid hydrolysis affored the desired 3-dimethyl analog (26). The alkylation of the key intermediate PhF-4-oxoproline methyl ester (18) with aldehydes was followed by the reaction sequence described above for the synthsis of compound 22, i.e., reduction, base hydrolysis, and a catalytic hydrogenation, led to 3-substitued analgous 33 and 34.

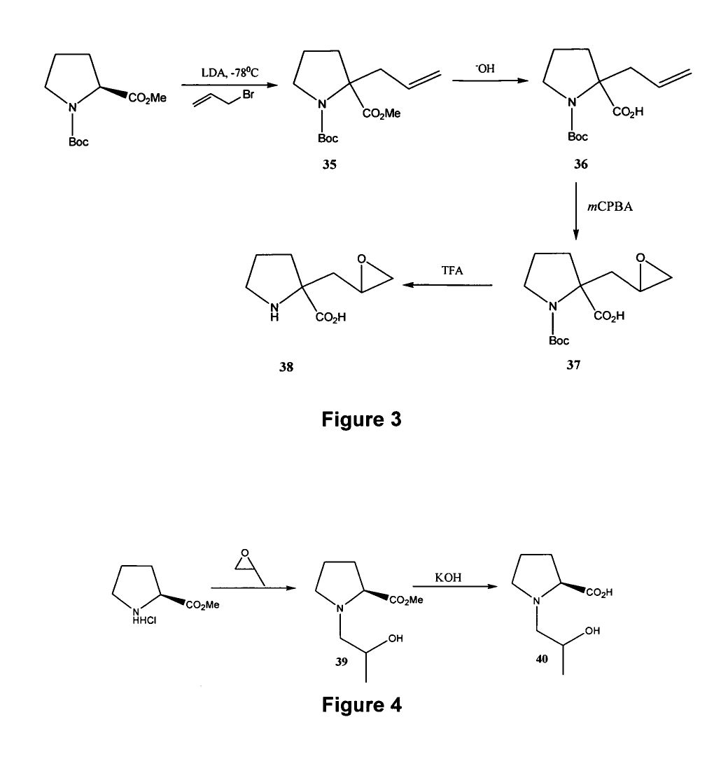

Boc-proline methyl ester was alkylated using allylbromide and LDA to give N-Boc-α-allylproline methyl ester (35), as shown in FIG. 3, which was subsequently converted to the free carboxylic acid (36) via basic hydrolysis. N-Boc-α-allylproline was then reacted with m-chloroperbenzoic acid to yield the epoxy-derivative (37). The removal of Boc-protecting group with TFA, followed by several lyophilizations to remove excess TFA yielded the desired α-oxiranylmethyl-proline analog (38).

The route to synthesis of compound 40 is shown in FIG. 4. Propylene oxide was used to neutralize the L-proline HCl salt. Exothermic reaction of propylene oxide with the acid salt led to further reaction of the epoxide with the amine moiety to form N-hydroxypropyl substituted amino acid (39). The base hydrolysis of compound 39 gave the desired acid (40).

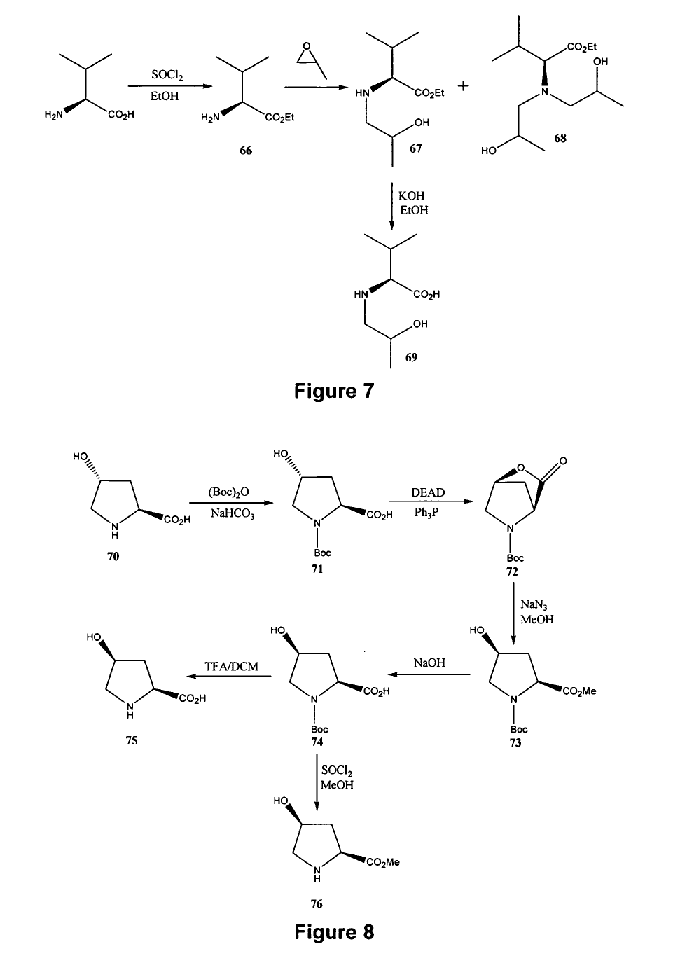

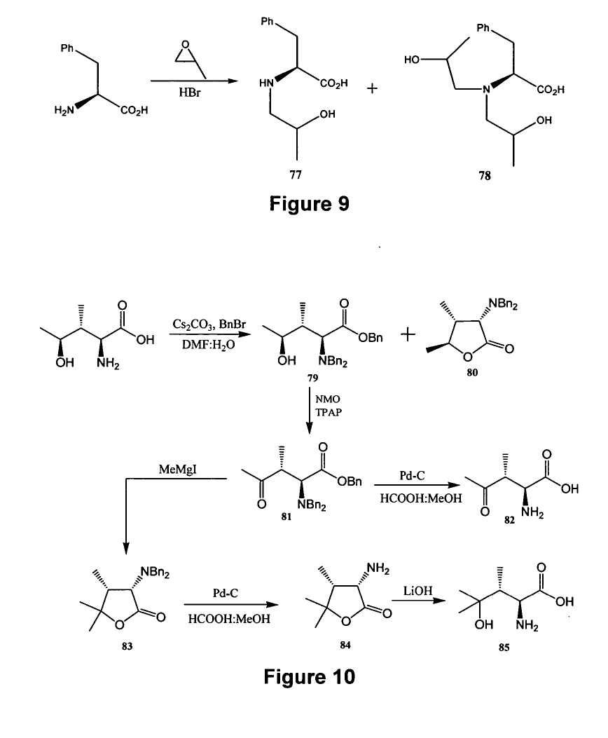

Similar reactivity of L-valine ethyl ester (66), synthesized from L-valine by reaction with thionyl chloride in ethanol, with propylene oxide led to the mono substituted amino acid (67) and also the di-substituted amino acid (68) (FIG. 7). The desired N-(2-hydroxypropyl)-L-valine (69) was isolated after base hydrolysis of mono substituted amino acid (67) (FIG. 7). Similar chemistry, shown in FIG. 9, depicts the one step synthesis of N-(2-hydroxypropyl)-L-phenylalanine (77). In this case L-phenylalanine was used as such i.e., acid moiety was not protected as an ester as in the case of valine compound 69. The disubstituted compound (78) was also observed as a by-product.

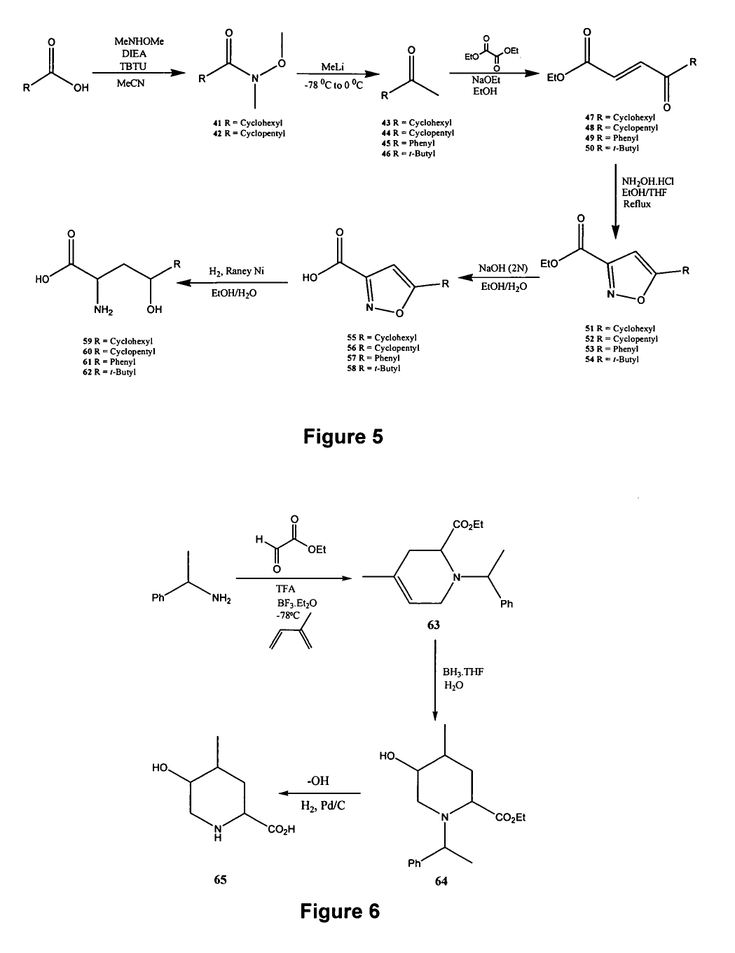

The analogs shown in FIG. 5 were prepared starting either from the corresponding acid or the ketone. For example, cyclohexyl acid, was transformed into a hydroxamate (41) from the reaction with TBTU and N-methyl O-methylhydroxylamine. The hydroxamate (41) was then converted into the ketone (43) by reaction with methyllithium. The reaction of this cyclohexyl methyl ketone (43) with diethyloxalate gave 4-cyclohexyl-2-hydroxy-4-oxo-but-2-enoic acid ethyl ester (47). The reaction of compound 47 with hydroxylamine led to oxazole intermediate (51). The base hydrolysis of 51 gave the acid (55) and which upon hydrogenolysis with Raney nickel gave the desired analog, 2-amino-4-cyclohexyl-4-hydroxy-butyric acid (59). The chemistry described above was repeated with the corresponding acid or the ketone to obtain analogs such as 2-amino-4-cyclopentyl-4-hydroxy-butyric acid (60), 2-amino-4-hydroxy-4-phenyl-butyric acid (61), and 2-amino-4-hydroxy-5,5-dimethyl-hexanoic acid (62).

Dipipecolic intermediate (63) was prepared from the condensation reaction of α-methyl benzylamine with ethylglyoxylate (FIG. 6). Hydroboration with BH₃. THF gave the protected form of 5-hydroxy-4-methyl-2-piperidine carboxylic acid (64). The hydrolysis and catalytic hydrogenolysis led to the isolation of 5-hydroxy-4-methyl-2-piperidine carboxylic acid (65).

The chirality of Boc protected trans-4-hydroxyproline (71) was inverted to compound 72 using Mitsunobu reaction conditions (Silverman et al., Org. Lett. 3: 2481-2484, 2001 and Org. Lett. 3: 2477, 2001) (FIG. 8). The hydrolysis of compound 72 to compound 73 to compound 74 and removal of Boc with TFA/DCM of intermediate 74 gave the desired compound 75. The methyl ester derivative of compound 75, i.e. compound 76 was prepared from 74 by reacting with thionyl chloride in methanol.

The protection of amino acid moiety of (2S,3R,4S)-4-hydroxyisoleucine was achieved in one step using Cs₂CO₃ as base, and BnBr in DMF/water mixture in good overall yield (FIG. 10). The reaction mixture contained mainly open chain compound (79), and some amount of the corresponding lactone (80). The oxidation of open chain intermediate (79), followed by hydrogenolysis gave the desire 4-keto analog (82) in a good yield. Grinyard addition of methyl magnesium iodide to the protected keto intermediate (81) gave dibenzyl lactone (83) in moderate yield. The deprotection using formic acid and Pd-C catalyst reaction conditions or hydrogenolysis gave the lactone (84) in good yield. Finally, the hydrolysis of lactone with LiOH affored the desired (2S,3R) analog 85 in an isolated yield of 90% (FIG. 10).

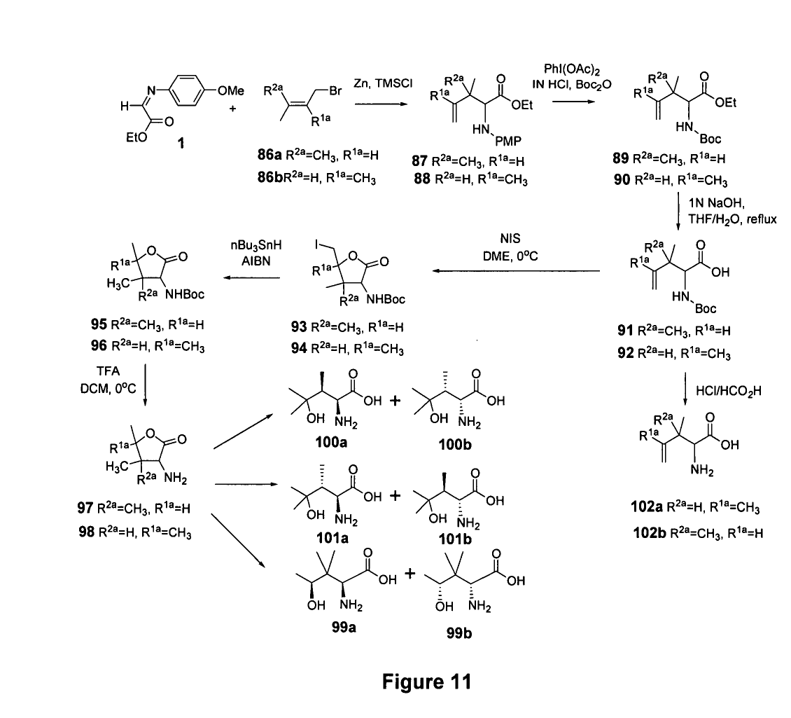

The analogs described in FIG. 11 were synthesized starting from a reaction of imine (1) either with 1-bromo-3-methylbut-2-ene or 1-bromo-2-methylbut-2-ene to give the condensation product 87 and 88, respectively. The removal of PMP group was accomplished with iodosobenzene diacetate, followed by in-situ protection of amino groups with Boc anhydride to yield 89 and 90, respectively. The hydrolysis of ester moiety, followed by reaction with N-iodosuccinimide in DME led to the iodolactone (compounds 93 and 94). nBuSnH and AlBN were to used to remove the iodo functional group, and subsequent removal of Boc group with TFA in dichloromethane gave the key lactone intermediate (compounds 97 and 98, respectively). The hydrolysis of 97 under basic conditions led to the isolation of an enantiomeric mixture (SS and RR isomers) of 99a and 99b. Similarly, base hydrolysis of compound 98 led to the isolation of compounds 100a and 100b (again, an enantiomeric mixture of SS and RR isomers), and 101a and 101b (an enantiomeric mixture of SR and RS isomers). The compounds 102a and 102b were obtained from compounds 92 and 91, respectively, by removal of Boc group under acidic conditions.

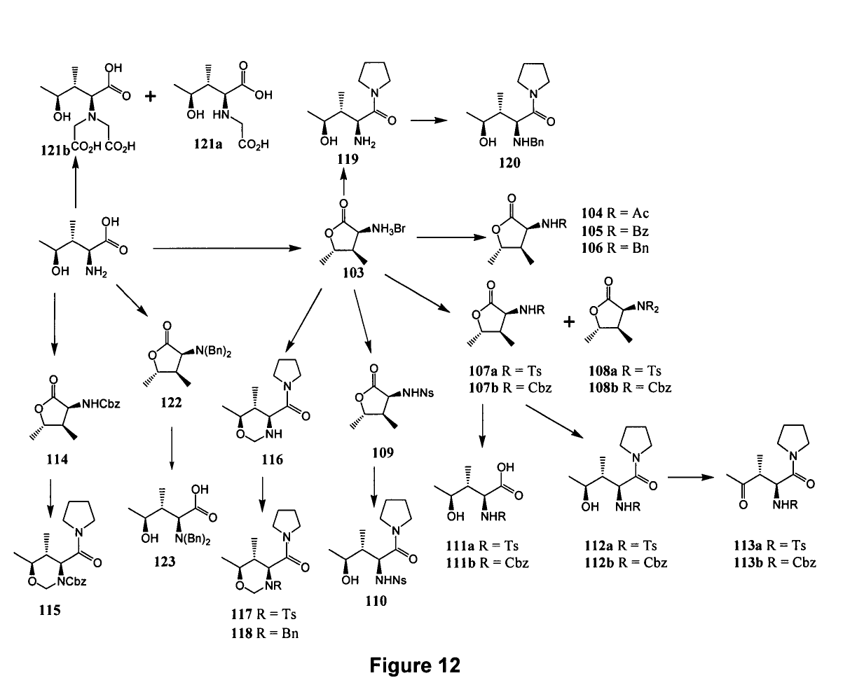

The compounds shown in FIG. 12 were either obtained starting from (2S,3R,4S)-4-hydroxyisoleucine or its lactone form (103). The direct derivatization of lactone (103) led to N-Ac (104), N-Bz (105), and N-Bn (106) derivatives. N-tosylate (107a) and N,N-ditosylate (108a) derivatives were isolated from a reaction mixture involving reaction of the lactone (103) with p-toluenesulfonyl chloride in dichloromethane in the presence of triethylamine. The base hydrolysis of mono tosylated lactone (107a) gave the N-Ts derivative (111a) of (2S,3R,4S)-4-hydroxyisoleucine, and similarly, reaction of compound 107a with pyrrolidine in dichloromethane led to the amide analog (112a). The oxidation of amide (112a) with PCC gave the corresponding 4-keto derivative (113a). The reaction of o-nitrobenzenesulfonyl chloride with lactone (103) led to N-Ns derivative (109), which upon further reaction with pyrrolidine in dichloromethane in the presence of triethylamine gave the corresponding N-Ns amide analog (110).

Surprisingly, the reaction of lactone (103) with pyrrolidine in dichloromethane gave a compound which showed extra methylene signals in ¹H NMR. It turned out to be a compound in which N and O are bridged with a —CH₂— group i.e., amide (116). It seems reasonable to conclude that the source of —CH₂— group is solvent, in this case, i.e., dichloromethane reacts with the intermediate. It also seems reasonable to propose that the opening of lactone to form an amide intermediate with pyrollidine was followed by the reaction of dichloromethane with N and O of the intermediate to afford compound 116. The bridged amide (116) was tosylated and benzylated to give the corresponding derivatives 117 and 118. The reaction of (2S,3R,4S)-4-hydroxyisoleucine with CbzCl gave the Cbz-lactone (114) in almost quantitative yield, which further, upon reaction with pyrrolidine, gave the substituted amide (115). The purification of a reaction mixture from the reaction of (2S,3R,4S)-4-hydroxyisoleucine with bromo ethyl acetate in TBME/water mixture, led to the isolation of monosubstituted diacid (121a) and disubstituted triacid (121b). N,N-dibenzyl derivative (123) of (2S,3R,4S)-4-hydroxyisoleucine was obtained from the hydrolysis of the corresponding lactone (122), which in turn was prepared from (2S,3R,4S)-4-hydroxyisoleucine in two steps.

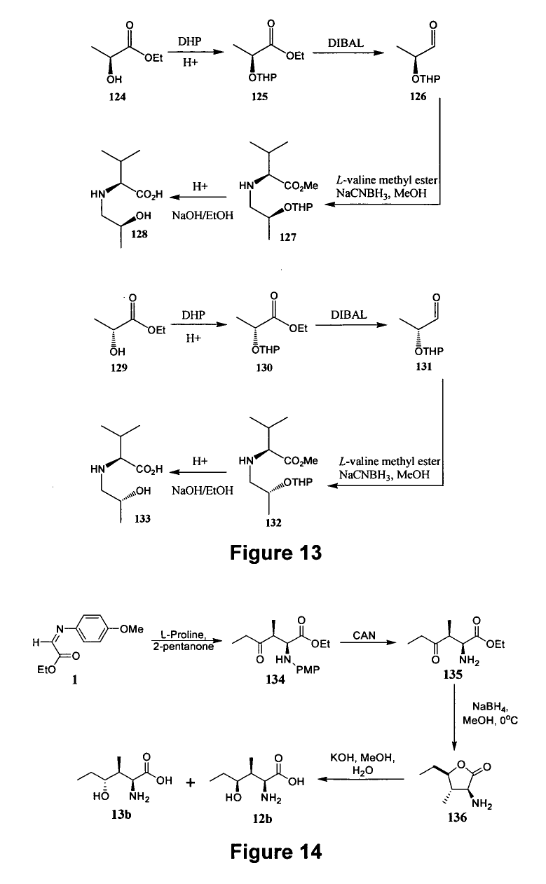

FIG. 13 depicts an enantioselecive synthesis of SS (128) and SR (133) derivatives. A diastereomeric mixture of these two compounds (compound 69) was synthesized using a different method and is given in FIG. 7. (S)-Lactic acid ethyl ester (124) reacted with DHP to give THP protected intermediate (124), which was reduced with DIBAL to give the aldehyde (126). The key transformation, reductive amination, of the aldehyde (126) with L-valine methyl ester hydrochloride and sodium cyanoborohydride gave the protected compound (127). The base hydrolysis to ester moiety to an acid, and removal of THP group with acid gave the desired SS-isomer (128) in an excellent overall yield. Above reaction sequence was repeated with (R)-Lactic acid ethyl ester to obtain SR-isomer (133), again in an excellent isolated yield.

FIG. 14 depicts the synthesis of two diastereoisomers and analog of (2S,3R,4S)-4-hydroxyisoleucine (12b & 13b). Mannich condensation reaction of imine (1) with 2-pentanone in the presence of L-proline gave the desired SS-keto intermediate (134). PMP groups was removed with ceric ammonium nitrate, followed by sodium borohydride reaction in methanol to give a lactone (136), as a mixture of two diastereoisomers. The base hydrolysis of the lactone and purification afforded the SSS-isomer (12b) and also the SSR-isomer (13b).

B) Detailed Experimental Procedures

Detailed reaction conditions used in the preparation of compounds I through 136 are as follows.

Synthesis of Compound 1

To a stirred solution of p-anisidine (50 g, 406 mmol) in toluene (400 mL) in a 1 liter round bottomed flask was added sodium sulfate (200 g, ˜2.5 eq). Ethyl glyoxalate (82 mL, 50% in toluene, 406 mmol) was added slowly to the above reaction mixture, and the mixture was stirred for 30 min. After this time, the sodium sulfate was filtered off using celite and toluene was removed under reduced pressure. Compound 1 (80 g, 95%) was isolated after drying and used as is for the next reaction.

### General Procedure for Asymmetric Condensation of Ketones with Imine (1)

Imine 1 (1 eq.) was added dropwise to a mixture of ketone (22 eq) and L-proline (0.35 eq) in dry DMSO (40 mL) at room temperature under nitrogen, and the mixture was stirred at room temperature for 2 h. The reaction mixture was diluted with phosphate buffer (pH 7.4), followed by extraction with ethyl acetate (3×200 mL). The organic phases were combined, dried over MgSO₄ and concentrated under reduced pressure. The desired compound (2) was isolated after purification by silica gel column chromatography. In few cases, excess ketone was removed under reduced pressure or by silica gel column chromatography.

### General Procedure for Isomerization of the Mannich Condensation Product (2)

To a solution (2S,3S) isomer (2) in minimum amount of the solvent was added 0.4 equivalent of DBN (1,4-diazabicyclo[4.3.0]non-5-ene), and the mixure was stirred at room temperature over night in an open flask. The solvent was evaporated by blowing a stream of argon over the reaction mixture. The crude mixture was redissolved in minimum amount of solvent and above procedure was repeated several times until the ratio of two diastereoisomers remained unchanged. The solvent was evaporated under reduced pressure, and the residue was purified using high resolution silica gel chromatography to obtain mainly (2S,3R) diastereoisomer.

The following compounds were prepared using the general procedures as described above.

### Synthesis of (2S,3S)-ethyl 2-(4-methoxyphenyl amino)-3-methyl-4-oxo-hexanoate (2b)

2b: yellow oil (72%). ¹H NMR (CDCl₃, 300 MHz): δ 1.04 (t, ³J (H₈, H₇)=7.2 Hz, 3H, H₈), 1.21 (t, ³J (H₁, H₂)=7.2 Hz, 3H, H₁), 1.24 (d, ³J (H₉, H₅)=7.2 Hz, 3H, H₉), 2.55 (q, ³J (H₇, H₈)=7.2 Hz 2H, H₇), 3.03 (m, 1H, H₅), 3.73 (s, 3H, H₁₇), 3.90 (brs, 1H, H₁₀), 4.15 (q, ³J (H₂, H₁)=7.2 Hz, 1H, H₂), 4.30 (m, 1H, H₄); 6.63-6.66 (d, ³J (H₁₂, H₁₃)=9.1 Hz, 2H, H₁₂, ₁₆), 6.75-6.78 (d, ³J (H₁₂, H₁₃)=9.1 Hz , 2H, H₁₃, H₁₅). ¹³C NMR (CDCl₃, 75 MHz): δ 7.53 (C₈), 12.51 (C₉), 14.08 (C₁), 34.32 (C₇), 48.37 (C₅), 55.59 (C₁₇), 59.65 (C₄), 61.43 (C₂), 114.71, 115.61 (C₁₂, C₁₃, C₁₅, C₁₆), 140.76 (C₁₁), 152.96 (C₁₄), 172.85 (C₃), 211.81 (C₆). MS m/z: 294 (M+1), 316 (M+23).

### Synthesis of (2S,3R)-ethyl 2-(4-methoxyphenyl amino)-3-methyl-4-oxo-hexanoate (3b)

3b: yellow oil (60%). ¹H NMR (CDCl₃, 300 MHz): δ 1.06 (t, ³J (H₈, H₇)=7.2 Hz, 3H, H₈), 1.22 (m, 6H, H₁, H₉), 2.55 (q, ³J (H₇, H₈)=7.2 Hz 2H, H₇), 3.03 (m, 1H, H₅), 3.73 (s, 3H, H₁₇), 3.90 (brs, 1H, H₁₀), 4.15 (q, ³J (H₂, H₁)=7.2 Hz, 1H, H₂), 4.26 (m, 1H, H₄), 6.63-6.66 (d, ³J (H₁₂, H₁₃)=9.1 Hz, 2H, H₁₂, H₁₆ ), 6.75-6.78 (d, ³J (H₁₂, H₁₃)=9.1 Hz, 2H, H₁₃, H₁₅). ¹³C NMR (CDCl₃, 75 MHz): δ 7.46 (C₈), 13.22 (C₉), 14.08 (C₁), 34.94 (C₇), 48.29 (C₅), 55.59 (C₁₇), 60.69 (C₄), 61.07 (C₂), 114.71, 115.77 (C₁₂, C₁₃, C₁₅, C₁₆), 140.70 (C₁₁), 153.03 (C₁₄), 172.68 (C₃), 212.10 (C₆). MS m/z: 294 (M+1), 316 (M+23).

### Synthesis of (S)-ethyl 2-(4-methoxyphenylamino)-2-((S)-2-oxo-cyclohexyl)-acetate (2e)

2e: brown oil (85%). ¹H NMR (CDCl₃, 200 MHz): δ 1.21 (t, ³J (H₁, H₂)=7.2 Hz, 3H, H₁), 1.65-2.49 (m, 8H, H₇, H₈, H₉, H₁₀), 2.81 (m, 1H, H₅), 3.74 (s, 3H, H₁₉), 3.87 (brs, 1H, H₁₁), 4.14 (q, ³J (H₂, H₁)=7.2 Hz, 1H, H₂), 4.23 (d, ³J (H₄, H₅)=5.3 Hz, 1H, H₄), 6.70-6.73 (d, ³J (H₁₃, H₁₄)=9.2 Hz, 2H, H₁₃, H₁₇), 6.75-6.78 (d, ³J (H₁₂, H₁₃)=9.2 Hz, 2H, H₁₄, H₁₆). ¹³C NMR (CDCl₃, 75 MHz): δ 14.08 (C₁), 24.71 (C₈), 26.81 (C₉), 29.54 (C₁₀), 41.78 (C₇), 53.50 (C₅), 55.64 (C₁₈), 58.05 (C₄), 61.08 (C₂); 114.70, 116.01 (C₁₃, C₁₄, C₁₆, C₁₇), 141.08 (C₁₂), 152.99 (C₁₅), 173.40 (C₃), 210.02 (C₆). MS (IC) m/z: 306 (M+1).

### Synthesis of (S)-ethyl 2-(4-methoxyphenylamino)-2-((R)-2-oxo-cyclohexyl)-acetate (3e)

3e: orange oil (60%, 98% purity). ¹H NMR (CDCl₃, 300 MHz): δ 1.22 (t, ³J (H₁, H₂)=7.2 Hz, 3H, H₁), 1.65-2.49 (m, 8H, H₇, H₈, H₉, H₁₀), 3.11 (m, 1H, H₅), 3.74 (s, 3H, H₁₈), 3.99 (d, ³J (H₄, H₅)=3.7 Hz, 1H, H₄), 4.15 (q, ³J (H₂, H₁)=7.2 Hz, 1H, H₂), 4.24 (brs, 1H, H₂), 4.24 (brs, 1H, H₁₁), 6.62-6.65 (d, ³J (H₁₃, H₁₄)=8.7 Hz, 2H, H₁₃, H₁₇), 6.75-6.78 (d, ³J (H₁₂, H₁₃)=8.7 Hz, 2H, H₁₄, H₁₆). ¹³C NMR (CDCl₃, 75 MHz): δ 14.04 (C₁), 24.47 (C₈), 26.77 (C₉), 30.45 (C₁₀), 41.73 (C₇), 53.51 (C₅), 55.61 (C₁₈), 58.99 (C₄), 61.09 (C₂), 114.67, 115.53 (C₁₃, C₁₄, C₁₆, C₁₇), 142.09 (C₁₂), 152.69 (C₁₅), 172.97 (C₃), 210.87 (C₆). MS (IC) m/z: 306 (M+1).

### Synthesis of (S)-ethyl 2-(4-methoxyphenylamino)-2-((S)-2-oxo-cycloheptyl)-acetate (2f)

2f: recrystallized from ethyl acetate, yellow solid (65%). ¹H NMR (CDCl₃, 200 MHz): 1.20 (t, ³J (H₁, H₂)=7.1 Hz, 3H, H₁), 1.31-2.02 (m, 8H, H₈, H₉, H₁₀, H₁₁), 2.52 (m, 2H, H₇), 2.92 (m, 1H, H₅), 3.73 (s, 3H, H₁₉), 3.92 (brs, 1H, H₁₂), 4.13 (q, ³J (H₂, H₁)=9 Hz, 2H, H₁₃, H₁₈), H₂), 4.26 (d, ³J (H₄, H₅)=5.9 Hz, 1H, H₄), 6.64-6.68 (d, ³J (H₁₄, H₁₈), 6.73-6.78 (d, ³J (H₁₄, H₁₅)=9 Hz, 2H, H₁₅, H₁₇). ¹³C NMR (CDCl₃, 75 MHz): δ 14.11 (C₁), 24.71, 27.12, 29.22, 29.80 (C₈, C₉, C₁₀, C₁₁), 43.86 (C₇), 55.16 (C₅), 55.64 (C₁₉), 60.62 (C₄), 61.17 (C₂), 114.72, 115.99 (C₁₄, C₁₅, C₁₇, C₁₈), 140.93 (C₁₃), 153.05 (C₁₆), 173.14 (C₃), 214.34 (C₆). MS (E) m/z: 342 (M+23).

### Synthesis of (S)-ethyl 2-(4-methoxyphenylamino)-2-((R)-2-oxo-cycloheptyl)-acetate (3f)

3f: yellow oil (99% purity). ¹H NMR (CDCl₃, 300 MHz): δ 1.23 (t, ³J (H₁, H₂)=7.2 Hz, 3H, H₁), 1.32-2.03 (m, 8H, H₈, H₉, H₁₀, H₁₁), 2.54 (m, 2H, H₇), 3.03 (m, 1H, H₅), 3.73 (s, 3H, H₁₉), 4.16 (q, ³J (H₂, H₁)=7.2 Hz, 1H, H₂), 4.29 (brs, 1H, H₁₂), 4.31 (d, ³J (H₄, H₅)=4.7 Hz, 1H, H₄), 6.66-6.69 (d, ³J (H₁₄, H₁₅)=9.1 Hz, 2H, H₁₄, H₁₈), 6.76-6.80 (d, ³J (H₁₄, H₁₅)=9.1 Hz, 2H, H₁₅, H₁₇). ¹³C NMR (CDCl₃, 75 MHz): δ 14.09 (C₁), 24.15, 27.11, 28.94, 29.82 (C₈, C₉, C₁₀, C₁₁), 43.80 (C₇), 54.29 (C₅), 55.62 (C₁₉), 60.60 (C₄), 61.21 (C₂), 114.79, 115.15 (₁₄, C₁₅, C₁₇, C₁₈), 140.92 (C₁₃), 152.66 (C₆), 172.50 (C₃), 214.09 (C₆). MS (E) m/z: 342 (M+23).

### Synthesis of (2S,3S)-ethyl 2-(4-methoxyphenyl amino)-4-methyl-3-phenylpentanoate (2c)

2c: recrystallization from hexane ether, yellow solid (75%). ¹H NMR (CDCl₃, 200 MHz): δ 1.25 (t, ³J (H₁, H₂)=7.1 Hz, 3H, H₁), 2.15 (s, 3H, H₇), 3.51 (brs, 1H, H₁₄), 3.74 (s, 3H, H₂₁), 4.19 (q, ³J (H₂, H₁)=7.1 Hz, 1 H, H₂), 4.25 (d, ³J (H₄, H₅)→8.5 Hz, 1H, H₄), 4.64 (d, ³J (H₅, H₄)=8.5 Hz, 1H, H₅), 6.58-6.62 (d, ³J (H₁₆, H₁₇)=9 Hz, H₁₆, H₂₀), 6.70-6.74 (d, ³J (H₁₆, H₁₇)=9 Hz, 2H, H₁₇, H₁₉), 7.24-7.37 (m, 5H, H₉, H₁₀, H₁₁, H₁₂, H₁₃). ¹³C (CDCl₃, 75 MHz): δ 14.09 (C₁), 29.19 (C₇), 55.60 (C₂₁), 59.78 (C₅) 61.29 (C₂), 61.53 (C₄), 114.49, 116.12 (C₁₆, C₁₇, C₁₉, C₂₀), 128.12 (C₁₁), 129.04. 129.19 (C₉, C₁₀, C₁₂, C₁₃), 134.34 (C₈), 140.61 (C₁₅), 153.01 (C₁₈), 173.22 (C₃), 206.09 (C₆). MS (E) m/z: 364 (M+23).

### Synthesis of (2S,3R)-ethyl 2-(4-methoxyphenyl amino)-4-methyl-3-phenylpentanoate (3c)

3c: yellow oil (90% purity). ¹H NMR (CDCl₃, 300 MHz): δ 0.88 (t, ³J (H₁, H₂)=7.1 Hz, 3H, H₁), 2.17 (s, 3H, H₇), 3.74 (s, 3H, H₂₁), 3.78 (brs, 1H, H₁₄), 3.84 (q, ³J (H₂H₁)=7.1 Hz, 1H, H₂), 4.11 (d, ³J (H₄, H₅)=8.7 Hz, 1H, H₄), 4.55 (d, ³J (H₅), 6.65-6.68 (d, ³J (H₁₆, H₁₇)=9 Hz, 2H, H₁₆, H₂₀), 6.72-6.75 (d, ³J (H₁₆, H₁₇)=9Hz, 2H, H₁₇, H₁₉), 7.32 (brs, 5H, H₉, H₁₀, H₁₁, H₁₂, H₁₃). ¹³C NMR (CDCl₃, 75 MHz): δ 13.31 (C₁), 29.53 (C₇), 55.11 (C₂₁), 60.40 (C₂) 61.07, 61.77 (C₄, C₅), 114.30, 116.19 (C₁₆, C₁₇, C₁₉, C₂₀), 127.77 (C₁₁), 128.63, 128.92 (C₉, C₁₀, C₁₂, C₁₃), 133.82 (C₈), 140.70 (C₁₅), 152.96 (C₁₈), 172.54 (C₃), 205.21 (C₆). MS (E) m/z: 364 (M+23).

### Synthesis of (2S,3S)-ethyl 3-benzyl-2-(4-methoxyphenyl amino)-4-oxopentanoate (2d)

2d: yellow solid (60%). ¹H NMR (CDCl₃, 300 MHz): δ 1.26 (t, ³J (H₁, H₂)=7.1 Hz, 3H, H₁), 2.04 (s, 3H, H₇), 3.09 (m, 2H, H₈), 3.34 (m, 1H, H₅), 3.75 (s, 3H, H₂₂), 4.08 (brs, 1H, H₁₅), 4.18 (q, ³J (H₂, H₁)=7.1 Hz, 1H, H₂), 4.19 (m, 1H, H₄), 6.49-6.52 (d, ³J (H₁₇, H₁₈)=9 Hz, 2H, H₁₇, H₂₁), 6.73-6.76 (d, ³J (H₁₇, H₁₈)=9 Hz, 2H, H₁₈, H₂₀), 7.24-7.37 (m, 5H, H₉, H₁₀, H₁₁, H₁₂, H₁₃). ¹³C (CDCl₃, 75 MHz) : δ 14.14 (C₁), 30.98 (C₇), 34.67 (C₈), 55.68 (C₂₂), 57.02 (C₅), 58.41 (C₄), 61.52 (C₂), 114.81, 115.32 (C₁₇, C₁₈, C₂₀, C₂₁), 126.69 (C₁₂), 128.64, 129.05 (C₁₀, C₁₁, C₁₃, C₁₄), 138.66 (C₉), 140.35 (C₁₆), 152.93 (C₂₂), 172.52 (C₃), 209.36 (C₆). MS (E) m/z: 356 (M+1), 378 (M+23).

### Synthesis of (2S,3R)-ethyl 3-benzyl-2-(4-methoxyphenyl amino)-4-oxopentanoate (3d)

3d: yellow oil (99% purity). ¹H NMR (CDCl₃, 300 MHz): δ 1.20 (t, ³J (H₁, H₂)=7.2 Hz, 3H, H₁), 2.08 (s, 3H, H₇), 2.98 (m, 2H, H₈), 3.43 (m, 1H, H₅), 3.74 (s, 3H, H₂₂), 4.13 (m, 3H, H₂, H₄), 4.45 (brs, 1H, H₁₅), 6.58-6.61 (d, ³J (H₁₇, H₁₈)=8.8 Hz, 2H, H₁₇, H₂₁), 6.76-6.79 (d, ³J (H₁₇, H₁₈)=8.8 Hz, 2H, H₁₈, H₂₀), 7.17-7.30 (m, 5H, H₉, H₁₀, H₁₁, H₁₂, H₁₃). ¹³C NMR (CDCl₃, 75 MHz): δ 13.93 (C₁), 31.01 (C₇), 34.53 (C₈), 55.33 (C₂₂), 55.67 (C₅), 58,79 (C₄), 60.99 (C₂), 114.48, 115.47 (C₁₇, C₁₈, C₂₀, C₂₁), 126.49 (C₁₂), 128.46, 128.79 (C₁₀, C₁₁, C₁₃, C₁₄), 138.02 (C₉), 140.70 (C₁₆), 152.73 (C₂₂), 172.75 (C₃), 209.77 (C₆). MS (E) m/z: 356 (M+1), 378 (M+23).

General Procedure for Deprotection of p-methoxypheny (PMP) Group of γ-oxo-α-(4-methoxyphenyl Amino) Esters with Ceric Ammonium Nitrate (CAN)

To a solution of γ-oxo-α-(4-methoxyphenyl amino) ester (10 mmol) in CH₃CN (6 mL) at 0° C., was added a solution of ceric ammonium nitrate (CAN, 3 eq) in water (60 mL) with added quickely but dropwise with stirring. The reaction mixture was stirred for 45 min at 0° C. CH₂Cl₂ (60 mL) was added to the reaction mixture and the phases were separated. The organic phase was washed with 0.1 N aqueous HCl (60 mL). The aqueous phases were combined and extracted with CH₂Cl₂ (3×130 mL), basified with a solution of Na₂CO₃ (2N) to pH 7 and extracted again with CH₂Cl₂ (3×150 mL). The combined organic phases were dried over MgSO₄ and concentrated under reduced pressure to obtain γ-oxo-α-aminoesters. Following compounds were prepared using the general procedures described above.

### Synthesis of (2S,3R)-ethyl 2-amino-3-methyl-4-oxopentanoate (6a)

6a: clear oil (88%). ¹H NMR (CDCl₃, 300 MHz): δ 1.16 (d, ³J (H₈, H₅)=7.5 Hz, 3H, H₈), 1.24 (t, ³J (H₁, H₂)=7.2 Hz, 3H, H₁), 1.70 (brs, 1H, H₉), 2.17 (s, 3H, H₇), 2.92 (m, 1H, H₅), 3.53 (d, ³J (H₄, H₅)=6.4 Hz, 1H, H₄), 4.16 (q, ³J (H₂, H₂) ¹³C NMR (CDCl₃, 75 MHz): δ 13.25 (C₈), 14.00 (C₁), 28.73 (C₇), 50.18 (C₅), 56.72 (C₄), 60.89 (C₂), 174.26 (C₃), 210.06 (C₆). MS (IC) m/z: 174 (M+1).

### Synthesis of (2S,3S)-ethyl 2-amino-3-methyl-4-oxopentanoate (4a)

4a: clear oil (88%). ¹H NMR (CDCl₃, 300 MHz): δ 1.11 (d, ³J (H₈, H₅)=7.1 Hz, 3H, H₈), 1.25 (t, ³J (H₁, H₂)=7.2 Hz, 3H, H₁), 1.70 (brs, 1H, H₉), 2.20 (s, 3H, H₇), 2.92 (m, 1H, H₅), 3.86 (d, ³J (H₄, H₅)=4.9 Hz, 1H, H₄), 4.16 (q, ³J (H₂, H₂). ¹³C (CDCl₃, 50 MHz): δ 10.82 (C₈), 14.07 (C₁), 28.24 (C₇), 49.64 (C₅), 55.26 (C₄), 61.16 (C₂), 174.18 (C₃), 209.80 (C₆). MS (IC) m/z: 174 (M+1).

### Synthesis of (2S,3S)-ethyl 2-amino-3-methyl-4-oxohexanoate (4b)

4b: clear oil (84%). ¹H NMR (CDCl₃, 300 MHz): δ 1.04 (t, ³J (H₈, H₇)=7.2 Hz, 3H, H₈), 1.11 (d, ³J (H₉, H₅)=7.2 Hz, 3H, H₉), 1.25 (t, ³J (H₁, H₂)=7.2 Hz, 3H, H₁), 2.52 (q, ³J (H₇, H₈)=7.2 Hz, 2H, H₇), 2.91 (m, 1H, H₅), 3.84 (d, ³J (H₄, H₅)=5.0 Hz, 1H, H₄), 4.16 (q, ³J (H₂, H₁)=7.2 Hz, 1H, H₂). ¹³C NMR (CDCl₃, 75 MHz): δ 7.58 (C₈), 11.23 (C₉), 14.09 (C₁), 34.03 (C₇), 48.74 (C₅), 55.45 (C₄), 61.10 (C₂), 174.15 (C₃), 212.44 (C₆). MS (IC) m/z: 188 (M+1).

### Synthesis of (2S,3R)-ethyl 2-amino-3-methyl-4-oxohexanoate (6b)

6b: clear oil (84%). ¹H NMR (CDCl₃, 300 MHz): δ 1.02 (t, ³J (H₈, H₇)=7.2 Hz, 3H, H₈), 1.14 (d, ³J (H₉, H₅)=7.2 Hz, 3H, H₉), 1.24 (t, ³J (H₁, H₂)=7.2 Hz, 3H, H₁), 2.50 (q, ³J (H₇, H₈)=7.2 Hz, 2H, H₇), 2.91 (m,1H, H₅), 3.53 (d, ³J (H₄, H₅)=6.5 Hz, 1H, H₄), 4.16 (q, ³J (H₂, H₁)=7.2 Hz, 1H, H₂). ¹³C NMR (CDCl₃, 75 MHz): δ 7.46 (C₈), 13.69 (C₉), 14.09 (C₁), 34.98 (C₇), 49.22 (C₅), 57.04 (C₄), 60.94 (C₂), 174.48 (C₃), 212.89 (C₆). MS (IC) m/z: 188 (M+1)

### Synthesis of (S)-ethyl 2-amino-2-((S)-2-oxocyclohexyl)acetate (4e)

4e: clear oil (80%). ¹H NMR (CDCl₃, 300 MHz): δ 1.26 (t, ³J (H₁, H₂)=7.2 Hz, 3H, H₁), 1.62-2.09 (m, 6H, H₈, H₉, H₁₀), 2.25-2.45 (m, 2H, H₇), 2.78 (m, 1H, H₅), 3.93 (d, ³J (H₄, H₅)=3.8 Hz, 1H, H₄), 4.17 (q, ³J (H₂, H₁)=7.2 Hz, 1H, H₂). ¹³C NMR (CDCl₃, 75 MHz): δ 14.14 (C₁), 24.68, 26.94, 27.68 (C₈, C₉, C₁₀), 41.94 (C₇), 53.44, 53.91 (C₄, C₅), 60.96 (C₂), 174.40 (C₃), 210.90 (C₆).

### Synthesis of (S)-ethyl 2-amino-2-((R)-2-oxocyclohexyl)acetate (6e)

6e: a clear oil (80%). ¹H NMR (CDCl₃, 300 MHz): δ 1.26 (t, ³J (H₁, H₂)=7.2 Hz, 3H, H₁), 1.62-2.09 (m, 6H, H₈, H₉, H₁₀), 2.25-2.45 (m, 2H, H₇), 2.98 (m, 1H, H₅), 3.35 (d, ³J (₄, H₅)=4.7 Hz, 1H, H₄), 4.17 (q, ³J (H₂, H₁)=7.2 Hz, 1H, H₂). ¹³C NMR (CDCl₃, 75 MHz): δ 14.14 (C₁), 24.87, 27.11, 30.76 (C₈, C₉, C₁₀), 41.94 (C₇), 53.70, 55.33 (C₄, C₅), 60.96 (C₂), 174.40 (C₃), 211.20 (C₆).

### Synthesis of (S)-ethyl 2-amino-2-((S)-2-oxocycloheptyl)acetate (4f)

4f: clear oil (80%). ¹H NMR (CDCl₃, 300 MHz): δ 1.26 (t, ³J (H₁, H₂)=7.2 Hz, 3H, H₁), 1.31-2.02 (m, 8H, H₈, H₉, H₁₀, H₁₁), 2.52 (m, 2H, H₇), 2.92 (m, 1H, H₅), 3.83 (d, ³J (H₄, H₅)=4.7 Hz, 1H, H₄), 4.18 (q, ³J (H₂, H₁)=7.2 Hz, 1H, H₂). ¹³C NMR (CDCl₃, 75 MHz): δ 14.15 (C₁), 23.92, 26.55, 29.57, 29.87 (C₈, C₉, C₁₀, C₁₁), 43.87 (C₇), 55.24, 56.08 (C₄, C₅), 61.03 (C₂), 174.58 (C₃), 214.71 (C₆).

### Synthesis of (S)-ethyl 2-amino-2-((R)-2-oxocyclohexptyl)acetate (6f)

6f: clear oil (80%). ¹H NMR (CDCl₃, 300 MHz): δ 1.28 (t, ³J (H₁, H₂)=7.2 Hz, 3H, H₁), 1.31-2.02 (m, 8H, H₈, H₉, H₁₀, H₁₁), 2.52 (m, 2H, H₇), 3.07 (m, 1H, H₅), 3.56 (d, ³J (H₄, H₅)=4.9 Hz, 1H, H₄), 4.18 (q, ³J (H₂, H₁)=7.2 Hz, 1H, H₂). ¹³C NMR (CDCl₃, 50 MHz): δ 13.95 (C₁), 23.67, 28.19, 29.23, 29.45 (C₈, C₉, C₁₀, C₁₁), 43.73 (C₇), 54.87, 57.20 (C₄, C₅), 60.78 (C₂), 174.23 (C₃), 214.33 (C₆).

### Synthesis of (2S,3S)-ethyl 2-amino-4-oxo-3-phenypentanoate (4c)

4c: clear oil (65%). ¹H NMR (CDCl₃, 200 MHz): δ 1.24 (t, ³J (H₁, H₂)=7.1 Hz, 3H, H₁), 1.47 (brs, 2H, H₁₄), 2.06 (s, 3H, H₇), 4.12 (m, 4H, H₂, H₅, H₄), 7.20-7.33 (m, 5H, H₉, H₁₀, H₁₁, H₁₂, H₁₃). ¹³C NMR (CDCl₃, 50 MHz): δ 13.85 (C₁), 29.03 (C₇), 55.79 (C₄), 60.92 (C₂), 62.20 (C₅), 127.86 (C₁₁), 128.85, 129.02 (C₉, C₁₀, C₁₂, C₁₃), 134.27 (C₈), 173.34 (C₃), 206.69 (C₆).

### Synthesis of (2S,3R)-ethyl 2-amino-4-oxo-3-phenypentanoate (6c)

6c: clear oil (65%). ¹H NMR (CDCl₃, 300 MHz): δ 0.91 (t, ³J (H₁, H₂)=7.1 Hz, 3H, H₁), 1.63 (brs, 2H, H₁₄), 2.08 (s, 3H, H₇), 3.93 (m, 4H, H₂, H₅, H₄), 7.18-7.31 (m, 5H, H₉, H₁₀, H₁₁, H₁₂, H₁₃). ¹³C NMR (CDCl₃, 75 MHz): δ 13.56 (C₁), 29.79 (C₇), 57.18 (C₄), 60.50 (C₂), 63.54 (C₅), 127.77 (C₁₁), 128.66, 128.91 (C₉, C₁₀, C₁₂, C₁₃), 134.73 (C₈), 134.73 (C₃), 206.59 (C₆).

### Synthesis of (2S,3S)-ethyl 2-amino-3-benzyl-4-oxopentanoate (4d)

4d: clear oil (50%). ¹H NMR (CDCl₃, 300 MHz): δ 1.26 (t, ³J (H₁, H₂)=7.2 Hz, 3H, H₁), 2.02 (s, 3H, H₇), 2.96 (m, 2H, H₈), 3.27 (m, 1H, H₅), 3.79 (d, ³J (H₄, H₅)=5.3 Hz, 1H, H₄), 4.13 (m, 1H, H₂), 7.14-7.31 (m, 5H, H₁₀, H₁₁, H₁₂, H₁₃, H₁₄). ¹³C NMR (CDCl₃, 75 MHz): δ 14.12 (C₁), 30.61 (C₇), 33.41 (C₈), 55.04 (C₅), 57.41 (C₄), 61.35 (C₂), 126.46 (C₁₂), 128.51, 128.97 (C₁₀, C₁₁, C₁₃, C₁₄), 138.95 (C₉), 173.83 (C₃), 209.71 (C₆).

### Synthesis of (2S,3R)-ethyl 2-amino-3-benzyl-4-oxopentanoate (6d)

6d: clear oil (50%). ¹H NMR (CDCl₃, 300 MHz): 1.27 (t, ³J (H₁, H₂)=7.2 Hz, 3H, H₁), 2.04 (s, 3H, H₇), 2.96 (m, 2H, H₈), 3.27 (m, 1H, H₅), 3.44 (d, ³J (H₄, H₅)=5.9 Hz, 1H, H₄), 4.17 (m, 1H, H₂), 7.17-7.33 (m, 5H, H₁₀, H₁₁, H₁₂, H₁₃, H₁₄). ¹³C NMR (CDCl₃, 75 MHz): δ 14.10 (C₁), 31.18 (C₇), 34.73 (C₈), 55.40 (C₅), 56.55 (C₄), 61.09 (C₂), 126.52 (C₁₂), 128.56, 128.84 (C₁₀, C₁₁, C₁₃, C₁₄), 138.62 (C₉), 174.78 (C₃), 210.43 (C₆).

General Procedure for the Hydrolysis of γ-oxo-α-aminoesters

To a solution of γ-oxo-α-aminoester in H₂O/MeOH (0.35 M) was added, dropwise, 2N aqueous KOH solution (1.1 equivalents), and the reaction mixture was stirred at room temperature for 24 h. An aqueous solution of 2N HCl acid was added to adjust the pH to 6. The solvents were evaporated under reduced pressure and the crude product was purified by silica gel column chromatography. Following compounds were prepared using the general procedures described above.

### Synthesis of (2S,3S)-2-amino-3-methyl-4-oxopentanoic acid (5a)

5a: an oil (50%). ¹H NMR (D₂O, 300 MHz): δ 1.26 (d, ³J (H₆, H₃)=7.5 Hz, 3H, H₆), 2.33 (s, 3H, H₅), 3.36 (m, 1H, H₃), 4.10 (d, ³J (H₂, H₃)=3.7 Hz, 1H, H₂). ¹³C NMR (D₂O, 50 MHz): δ 10.85 (C₆), 28.15 (C₅), 46.61 (C₃), 55.17 (C₂), 173.48 (C₁), 214.76 (C₄).

### Synthesis of (2S,3R)-2-amino-3-methyl-4-oxopentanoic acid (7a)

7a: an oil (56%). ¹H NMR (D₂O, 300 MHz): δ 1.31 (d, ³J (H₆, H₃)=7.5 Hz, 3H, H₆), 2.30 (s, 3H, H₅), 3.36 (m, 1H, H₃), 3.95 (d, ³J (H₂, H₃)=5.1 Hz, 1H, H₂). ¹³C NMR (D₂O, 50 MHz): δ 12.48 (C₆), 28.38 (C₅), 46.76 (C₃), 56.39 (C₂), 173.32 (C₁), 214.54 (C₄).

### Synthesis of (2S,3S)-2-amino-3-methyl-4-hexanoic acid (5b)

5b: an orange oil (80%). ¹H NMR (D₂O, 200 MHz): δ 1.02 (t, ³J (H₆, H₅)=6.9 Hz, 3H, H₆), 1.21 (d, ³J (H₇, H₃)=7.5 Hz, 3H, H₇), 2.67 (m, 2H, H₅), 3.35 (m, 1H, H₃), 4.04 (d, ³J (H₂, H₃)=4.1 Hz. 1H. H₂). ¹³C NMR (D₂O. 50 MHz): δ 7.30 (C₆), 11.20 (C₇), 34.56 (C₅), 45.64 (C₃), 56.72 (C₂), 173.53 (C₁), 217.49 (C₄).

### Synthesis of (2S,3R)-2-amino-3-methyl-4-hexanoic acid (7b)

7b: orange oil (80%). ¹H NMR (D₂O. 200 MHz): 6 1.02 (m, 3H, H₆), 1.29 (d, ³J (H₇, H₃)=7.5 Hz, 3H, H₇), 2.67 (m, 2H, H₅), 3.35 (m, 1H, H₃), 3.89 (d, ³J (H₂, H₃)=4.7 Hz, 1H, H₂), ¹³C NMR (D₂O, 50 MHz): δ 7.30 (C₆), 12.99 (C₇), 34.75 (C₅), 45.64 (C₃), 55.50 (C₂), 173.32 (C₁), 217.70 (C₄).

### Synthesis of (S)-2-amino-2-((S)-2-cyclohexyl)acetic acid (5e)

5e: yellow oil (63%). ¹H NMR (D₂O, 300 MHz): δ 1.72 (m, 4H, H₆, H₇), 1.89-2.17 (m, 4H, H₅, H₈), 2.54 (m, 1H, H₃), 3.25 (m, 1H, H₃), 4.17 (d, ³J (H₂, H₃)=2.2 Hz, 1H, H₂), ¹³C NMR (D₂O, 50 MHz): δ 24.54 (C₆), 27.10 (C₇), 27.87 (C₈), 41.74 (C₅), 50.75 (C₂), 53.66 (C₃), 173.66 (C₁), 215.30 (C₄).

### Synthesis of (S)-2-amino-2-((R)-2-cyclohexyl)acetic acid (7e)

7e: oil (63%). ¹H NMR (D₂O, 300 MHz): δ 1.72 (m, 4H, H₆, H₇), 1.89-2.17 (m, 4H, H₅, H₈), 2.54 (m, 1H, H₃), 3.25 (m, 1H, H₃), 3.74 (d, ³J (H₂, H₃)=4.9 Hz, 1H, H₂). ¹³C NMR (D₂O, 50 MHz): δ 24.76 (C₆), 27.44 (C₇), 31.34 (C₈), 42.06 (C₅), 50.75 (C₂), 55.14 (C₃), 173.66 (C₁), 215.54 (C₄).

### Synthesis of (S)-2-amino-2-((S)-2-cycloheptyl)acetic acid (5f)

5f: clear oil (70%). ¹H NMR (D₂O, 300 MHz): δ 1.31-2.01 (m, 8H, H₆, H₇, H₈, H₉), 2.45-2.77 (m, 2H, H₅), 3.43 (m, 1H, H₃), 4.05 (d, ³J (H₂, H₃)=2.6 Hz, 1H, H₂). ¹³C NMR (D₂O, 75 MHz): δ 23.22, 25.97, 29.29, 29.71 (C₆, C₇, C₈, C₉); 43.48 (C₅), 51.64 (C₃), 55.96 (C₂), 173.73 (C₁), 219.05 (C₄).

### Synthesis of (S)-2-amino-2-((R)-2-cycloheptyl)acetic acid (7f)

7f: clear oil (70%). ¹H NMR (D₂O, 300 MHz): δ 1.31-2.01 (m, 8H, H₆, H₇, H₈, H₉), 2.45-2.77 (m, 2H, H₅), 3.43 (m, 1H, H₃), 3.87 (d, ³J (H₂, H₃)=4.1 Hz, 1H, H₂). ¹³C NMR (D₂O, 75 MHz): δ 23.22, 27.91, 28.93, 29.26 (C₆, C₇, C₈, C₉), 43.79 (C₅), 51.39 (C₃), 57.39 (C₂), 173.53 (C₁), 219.52 (C₄).

### Synthesis of (2S,3S)-2-amino-4-oxo-3-phenylpentanoic acid (5c)

5c: clear oil (60%). ¹H NMR (D₂O, 300 MHz): δ 2.20 (s, 3H, H₅), 4.08 (d, ³J (H₂, H₃)=6.8 Hz, 1 H, H₂), 4.59 (d, ³J (H₃, H₂)=6.8 Hz, 1H, H₃), 7.28-7.49 (m, 5H, H₇, H₉, H₁₀, H₁₁). ¹³C NMR (D₂O, 75 MHz): δ 29.12 (C₅), 57.28 (C₂), 58.55 (C₃), 128.68 (C₉), 129.73, 130.05 (C₇, C₈, C₁₀ C₁₁), 133.44 (C₆), 173.43 (C₁), 211.17 (C₄).

### Synthesis of (2S,3R)-2-amino-4-oxo-3-phenylpentanoic acid (7c)

7c: clear oil (60%). ¹H NMR (D₂O, 300 MHz): δ 2.23 (s, 3H, H₅), 4.37 (d, ³J (H₂, H₃)=6.1 Hz, 1H, H₂), 4.57 (d, ³J (H₃, H₂)=6.1 Hz, 1H, H₃), 7.28-7.49 (m, 5H, H₇, H₈, H₉, H₁₀, H₁₁). ¹³C NMR (D₂O, 75 MHz): δ 29.13 (C₅), 56.01 (C₂), 58.94 (C₃), 129.20 (C₉), 129.50, 130.13 (C₇, C₈, C₁₀, C₁₁), 132.03 (C₆), 173.43 (C₁), 211.17 (C₄).

### Synthesis of (2S,3S)-2-amino-3-benzyl-4-oxopentanoic acid (5d)

5d: clear oil (70%). ¹H NMR (D₂O, 300 MHz): δ 2.01 (s, 3H, H₅), 2.96 (m, 2H, H₆), 3.61 (m, 1H, H₃), 4.01 (m, 1H, H₂), 7.29-7.46 (m, 5H, H₈, H₉, H₁₀, H₁₁, H₁₂). ¹³C NMR (D₂O, 75 MHz): δ 31.10 (C₅), 33.69 (C₆), 54.10 (C₃), 55.59 (C₂), 127.40 (C₁₀), 129.32, 129.43 (C₈, C₉, C₁₁, C₁₂), 138.07 (C₇), 173.82 (C₁), 214.92 (C₄).

### Synthesis of (2S,3R)-2-amino-3-benzyl-4-oxopentanoic acid (7d)

7d: clear oil (70%). ¹H NMR (D₂O, 300 MHz): δ 2.10 (s, 3H, H₅), 2.92-3.20 (m, 2H, H₆), 3.76 (m, 1H, H₃), 3.81 (m, 1H, H₂), 7.29-7.46 (m, 5H, H₈, H₉, H₁₀, H₁₁, H₁₂). ¹³C NMR (D₂O, 75 MHz): δ 30.97 (C₅), 34.35 (C₆), 53.77 (C₃), 55.59 (C₂), 127.54 (C₁₀), 129.22, 12932 (C₈, C₉, C₁₁, C₁₂), 137.91 (C₇), 173.37 (C₁), 215.26 (C₄).

General Methods for the Reduction of γ-oxo-α-amino-esters

General One Step Process Involving Deprotection-Reduction of γ-oxo-α-amino-esters:

To a solution of γ-oxo-α-amino-esters (10 mmol) in MeCN (6 mL) was added a solution of CAN (3 equivalents) in water (60 mL) quickly but dropwise, while keep temperature of the reaction mixture at 0° C. The reaction mixture was stirred at 0° C. for 45 min. Dichloromethane (60 mL) was added to the reaction mixture and the phases were separated. The organic phase was washed with an HCl aqueous solution (0.1N, 60 mL), and aqueous phases were combined and washed twice with dichloromethane. The aqueous phase was basified with an aqueous solution of Na₂CO₃ (2 N) to pH 7, and cooled to 0° C. To the above solution was added NaBH₄ (1.5 equivalents) and mixture was stirred at 0° C. for 90 min. The reaction mixture was extracted with dichloromethane (3×200 mL). The organic phases were combined, dried over MgSO₄, and concentrated under reduced pressure. The crude products contiaing amino lactones or γ-hydroxy-α-amino-esters were purified by silica gel column chromatogaphy to obtain the pure compounds.

General Procedure for Reduction of γ-oxo-α-aminoesters with Sodium Borohydride:

To a solution of γ-oxo-α-amino-esters (10 mmol) in MeCN (6 mL) was added NaBH₄ (1.2 equivalents) and the reaction mixture was stirred for 90 min. Water (40 mL) was added to neutralize the excess hydride, followed by addition of dichloromethane (40 mL). After separating the phases, the aqueous phase was extracted with dichloromethane (2×50 mL). The organic phases were combined, dried over MgSO₄ and concentrated under reduced pressure. The crude γ-hydroxy-α-amino-esters were purified by silica gel column chromatography to obtain pure products.

General Procedure for Reduction of γ-oxo-α-amino-esters with Sodium Borohydride and CeCl₃.7H₂O:

To a solution of γ-oxo-α-amino-esters (10 mmol) in MeOH (30 mL) at 0° C. was added CeCl₃.7H₂O (0.4 equivalent). The reaction mixture was stirred for 5 min. at 0° C., followed by addition of NaBH₄ (1.2 equivalent), and stirring for 90 min. Water (40 mL) was added to neutralized the excess hydride, followed by addition of dichloromethane (40 mL). After separating the phases, the aqueous phase was extracted with dichloromethane (2×50 mL). The organic phases were combined, dried over MgSO₄ and concentrated under reduced pressure. The crude γ-hydroxy-α-amino-esters were purified by silica gel column chromatography to obtain pure products.

General Procedure for Reduction of γ-oxo-α-aminoesters with Raney Nickel:

To a solution of γ-oxo-α-amino-esters (10 mmol) in MeOH (30 mL) at room temperature many spatulas of commercially available Raney Nickel were added to obtain a grey-black solution, and the reaction mixture was stirred vigorously. The reaction mixture was cooled to 0° C. and purged with hydrogen gas. The reaction mixture was stirred under hydrogen atmosphere (1 atm) at room temperature for 24 h. The crude reaction mixture was filtered through celite, followed by purification of the complex reaction mixture, containing amino lactones and/or γ-hydroxy-α-amino-esters, by silica gel column chromatography to obtain pure products.

The following compounds were prepared using the general procedures described above.

### Synthesis of Compound 8b

8b: Follwing a one step deprotection-reduction sequence, a diastereomeric mixture was obtained, 56%, as a clear oil. ¹H NMR (CDCl₃, 300 MHz): δ 0.77 (d, ³J (H₆, H₅)=7.2 Hz, 3H, H₆) , 0.91 (t, ³J (H₉, H₈)=7.2 Hz, 3H, H₉), 1.25 (t, ³J (H₁, H₂)=7.2 Hz, 3H, H₁), 1.31-1.59 (m, 1H, H₇), 1.99 (m, 1H, H₅), 3.62 (d, ³J (H₄, H₅)=2.8 Hz, 1H, H₄), 3.78 (m, 1H, H₇), 4.16 (q, ³J (H₂, H₁)=7.2 Hz, 2H, H₂).

### Synthesis of Compound 9b

9b: Following either a one step deprotection-reduction sequence or reduction of unprotected ethyl esters, a diastereomeric mixture was obtained, 40%, as a clear oil. ¹H NMR (CDCl₃, 300 MHz): δ 1.07 (t, ³J (H₈, H₇)=7.5 Hz, 3H, H₈), 1.23 (d, ³J (H₅, H₄)=5.3 Hz, 3H, H₅), 1.63 (m, 1H, H₄), 1.85 (m, 1H, H₇), 3.24 (d, ³J (H₂, H₄)=11.3 Hz, 1H, H₂), 3.91 (m, 1H, H₆). ¹H NMR (CDCl₃, 300 MHz): δ 1.06 (t, ³J (H₈, H₇)=7.2 Hz, 3H, H₈), 1.17 (d, ³J (H₅, H₄)=6.8 Hz, 3H, H₅), 1.43-1.67 (m, 1H, H₇), 2.34 (m, 1H, H₄), 3.26 (d, ³J (H₂, H₄)=10.5 Hz, 1H, H₂), 4.41 (m, 1H, H₆), MS (IC) m/z: 144 (M+1).

### Synthesis of Compound 8e

8e: Following either a one step deprotection-reduction sequence or reduction of unprotected ethyl esters with Raney Nickel, a diastereomeric mixture was obtained, 56%, as a clear oil. ¹H NMR (CDCl₃, 200 MHz): δ 1.23 (t, ³J (H₁, H₂)=7.1 Hz, 3H, H₁), 1.15-1.98 (m, 9H, H₅, H₇, H₈, H₉, H₁₀), 3.15 (brs, 3H, H₁₁, H₁₂), 3.46 (m, 1H, H₆), 3.61 (d, ³J (H₄₁, H₅)=2.7 Hz, 1H, H₄₁), 3.91 (d, ³J (H₄₂, H₅)=2.9 Hz, 1H, H₄₂), 4.14 (q, ³J (H₂, H₁)=7.1 Hz, 2H, H₂). ¹³C₁ NMR (CDCl₃, 50 MHz): δ 14.11 (C₁), 19.17, 25.33, 25.61 (C₈, C₉, C₁₀), 33.01 (C₇), 42.33 (C₅), 58.69 (C₄), 61.09 (C₂), 70.77 (C₆), 174.47 (C₃), ¹³C₂ NMR (CDCl₃, 50 MHz) δ 14.11 (C₁), 24.65, 25.07, 25.33 (C₈, C₉, C₁₀), 35.57 (C₇), 47.83 (C₅), 54.51 (C₄), 60.84 (C₂), 70.22 (C₆), 175.10 (C₃).

### Synthesis of compound (3S,3aS,8aS)-3-amino-octahydrocyclohepta[b]furan-2-one (9f-SSS)

9f (SSS): Following one step deprotection-reduction sequence was obtained, 68%, as a clear oil. ¹H NMR (CDCl₃, 300 MHz): δ 1.12-2.37 (m, 10H, H₄, H₅, H₆, H₇, H₈), 2.40 (m, 1H, H₃), 3.30 (d, ³J (H₂, H₃)=10.9 Hz, 1H, H₂), 4.51 (m, 1H, H₉). ¹³C NMR (CDCl₃, 75 MHz): δ 25.59, 25.70, 29.59, 30.67, 30.73 (C₄, C₅, C₆, C₇, C₈), 46.47 (C₃), 56.22 (C₂), 82.81 (C₉), 178.30 (C₁).

### Synthesis of Compound (3S,3aS,8aR)-3-amino-octahydrocyclohepta[b]furan-2-one (9f-SSR)

9f (SSR): Following Raney Nickel reduction of amion ester intermediate, 55%, a clear oil was obtained. ¹H NMR (CDCl₃, 300 MHz): δ 1.10-2.25 (m, 11H, H₃, H₄, H₅, H₆, H₇, H₈), 3.23 (d, ³J (H₂, H₃)=11.5 Hz, 1H, H₂), 4.02 (m, 1H, H₉). ¹³C NMR (CDCl₃, 75 MHz): δ 24.24, 25.28, 27.11, 28.47, 32.78 (C₄, C₅, C₆, C₇, C₈), 50.42 (C₃), 58.23 (C₂), 82.04 (C₉), 178.04 (C₁).

### Synthesis of Compound (3S,4S,5S)-3-amino-5-methyl-4-phenyl-dihydrofuran-2(3H)-one (9c-SSS)

9c (SSS): Obtained either from a one step deprotection-reduction step or from reduction of amino ester with NaBH₄ or NaBH₄/CeCl₃.7H₂O, 37%, as a clear oil. ¹H NMR (CDCl₃, 200 MHz): δ 0.99 (d, ³J (H₅, H₄)=6.6 Hz, 3H, H₅), 1.57 (brs, 2H, H₁₂), 3.62 (dd, ³J (H₃, H₂)=11.7 Hz, ³J (H₃, H₄)=8.1 Hz, 1H, H₃), 4.09 (d, ³J (H₂, H₃)=11.7 Hz, 1H, H₂), 4.86 (quint, ³J (H₄, H₅)=³J (H₄, H₃)=7.1 Hz, 1H, H₄), 7.21-7.37 (m, 5H, H₇, H₈, H₉, H₁₀, H₁₁), ¹³C NMR (CDCl₃, 50 MHz): δ 16.88 (C₅), 52.07, 52.60 (C₂, C₃), 77.10 (C₄), 127.76, 128.96 (C₇, C₈, C₉, C₁₀, C₁₁), 135.11 (C₆), 177.66 (C₁).

### Synthesis of Compound (3S,4S,5R)-3-amino-5-methyl-4-phenyl-dihydrofuran-2(3H)-one (9c-SSR)

9c (SSR): Obtained from a reduction of amino ester with Raney Nickel, 37%, as a clear oil. ¹H NMR (CDCl₃, 300 MHz): δ 1.41 (d, ³J (H₅, H₄)=6.0 Hz, 3H, H₅), 1.76 (brs, 2H, H₁₂), 2.93 (t, ³J (H₃, H₂)=³J (H₃, H₄)=11.1 Hz, 1H, H₃), 3.94 (d, ³J (H₂, H₃)=12.1 Hz, 1H, H₂), 4.53 (m, 1H, H₄), 7.27-7.41 (m, 5H, H₇, H₈, H₉, H₁₀, H₁₁). ¹³C NMR (CDCl₃, 75 MHz): δ 18.48 (C₅), 58.63, 59.11 (C₂, C₃), 78.79 (C₄), 127.56, 129.08 (C₇, C₈, C₁₀, C₁₁), 127.68 (C₉), 135.80 (C₆), 176.60 (C₁).

### Synthesis of a Compound 9d

9d: Obtained from a one step deprotection-reduction sequence, 1:1 diastereomeric mixture, 68%, as a clear oil. ¹H. NMR (CDCl₃, 300 MHz): δ 1.25 (d, ³J (H₁₂, H₁₁)=6.0 Hz, 3H, H₁₂), 2.14 (m, 1H, H₃), 2.74-3.11 (m, 2H, H₄), 3.45 (d, ³J (H₂, H₃)=11.3 Hz, 1H, H₂), 4.20 (m, 1H, H₁₁), 7.20-7.37 (m, 5H, H₆, H₇, H₈, H₉, H₁₀). ¹³C₁ NMR (CDCl₃, 75 MHz): δ 19.17 (C₁₂), 35.98 (C₄), 53.34 (C₃), 56.42 (C₂), 78.01 (C₁₁), 126.64 (C₈), 128.58, 128.85 (C₆, C₇, C₉, C₁₀), 138.05 (C₅), 177.32 (C₁). ¹H₂ NMR (CDCl₃, 300 MHz): δ 1.33 (d, ³J (H₁₂, H₁₁)=6.8 Hz, 3H, H₁₂), 2.72 (m, 1H, H₃), 2.74-3.11 (m, 2H, H₄), 3.52 (d, ³J (H₂, H₃)=10.9 Hz, 1H, H₂), 4.66 (m, 1H, H₁₁), 7.20-7.37 (m, 5H, H₆, H₇, H₈, H₉, H₁₀). ¹³C₂ NMR (CDCl₃, 75 MHz): δ 15.92 (C₁₂), 33.88 (C₄), 47.89 (C₃), 53.91 (C₂), 76.12 (C₁₁), 126.44 (C₈), 128.21, 128.58 (C₆, C₇, C₉, C₁₀), 137.51 (C₅), 177.76 (C₁).

### Synthesis of a Compound 11b

11b: Obtained from a one step deprotection-reduction sequence or reduction of the amino ethyl ester, a diastereomeric mixture, 40%, as a clear oil. ¹H₁ NMR (CDCl₃, 300 MHz): δ 1.03 (m, 6H, H₈, H₅), 1.51-1.75 (m, 2H, H₇, H₄), 3.73 (d, ³J (H₂, H₄)=7.8 Hz, 1H, H₂), 3.86 (m, 1H, H₆). ¹H₂ NMR (CDCl₃, 300 MHz): δ 0.90 (d, ³J (H₅, H₄)=7.2 Hz, 3H, H₅), 104 (t, ³J (H₈, H₇)=7.5 Hz, 3H, H₈), 1.56-1.84 (m, 1 H, H₇), 2.57 (m, 1H, H₄), 3.83 (d, ³J (H₂, H₄)=6.9 Hz, 1H, H₂), 4.26 (m, 1H, H₆). ¹³C₂ NMR (CDCl₃, 50 MHz): δ 6.45 (C₈), 9.84 (C₅), 23.08 (C₇), 38.15 (C₄), 56.14 (C₂), 81.73 (C₆), 178.45 (C₁). MS (IC) m/z: 144 (M+1).

### Synthesis of (S)-ethyl 2-amino-2-((1R,2S)-2-hydroxycyclohexyl)acetate (8e-SSR)

8e (SSR): Obtained from a one step deprotection-reduction sequence, 62%, as a clear oil. ¹H NMR (CDCl₃, 300 MHz): δ 1.24 (t, ³J (H₁, H₂)=7.2 Hz, 3H, H₁), 1.00-1.91 (m, 9H, H₅, H₇, H₈, H₉, H₁₀), 3.49 (m, 5H, H₁₁, H₁₂, H₆, H₄), 4.13 (q, ³J (H₂, H₁)=7.2 Hz, 2H, H₂). ¹³C NMR (CDCl₃, 75 MHz): δ 14.07 (C₁), 24.09, 25.28, 27.78 (C₈, C₉, C₁₀), 34.94 (C₇), 46.96 (C₅), 60.37 (C₄), 60.70 (C₂), 75.19 (C₆), 174.65 (C₃).

### Synthesis of Compound 11f

11f: A diastereomeric mixture of amino lactones was obtained either from one step deprotection-reduction sequence or reduction of the corresponding amino ester with Raney Nickel, 72%, was obtained as a clear oil. ¹H₁ NMR (CDCl₃, 200 MHz): δ 1.18-2.55 (m, 11H, H₃, H₄, H₅, H₆, H₇, H₈), 3.82 (d, ³J (H₂, H₃)=8.1 Hz, 1H, H₉). ¹³C₁ NMR (CDCl₃, 50 MHz): δ 20.63, 21.38, 28.40, 30.45, 31.15 (C₄, C₅, C₆, C₇, C₈), 45.51 (C₃), 54.68 (C₂), 80.28 (C₉), 178.44 (C₁). ¹H₂ NMR (CDCl₃, 200 MHz): δ 1.18-2.57 (m, 11H, H₄, H₅, H₆, H₇, H₈, H₃), 3.61 (d, ³J (H₂, H₃)=6.8 Hz, 1H, H₂), 4.44 (m, 1H, H₉). ¹³C₂ NMR (CDCl₃, 50 MHz): δ 22.90, 24.30, 25.42, 26.71, 33.10 (C₄, C₅, C₆, C₇, C₈), 46.00 (C₃), 54.68 (C₂), 83.80 (C₉), 177.94 (C₁).

### Synthesis of (2S,3R,4R)-ethyl 2-amino-4-hydroxy-3-phenylpentanoate (10c-SRR)

10c (SRR): Obtained from one step deprotection-reduction sequence, 60%, as a clear oil. ¹H NMR (CDCl₃, 200 MHz): δ 1.02 (t, ³J (H₁, H₂)=7.1 Hz, 3H, H₁), 1.09 (d, ³J (H₇, H₆)=6.4 Hz, 3H, H₇), 2.59 (brs, 3H, H₁₄, H₁₅), 2.93 (dd, ³J (H₅, H₆)=3.2 Hz, ³J (H₅, H₄)=8.1 Hz, 1H, H₅), 3.98 (q, ³J (H₂, H₁)=7.1 Hz, 2H, H₂), 4.00 (d, ³J (H₄, H₅)=8.1 Hz, 1H, H₄), 4.34 (m, 1H, H₆), 7.06-7.33 (m, 5H, H₉, H₁₀, H₁₁, H₁₂, H₁₃). ¹³C NMR (CDCl₃, 50 MHz): δ 13.70 (C₁), 20.40 (C₇), 54.40 (C₅), 57.14 (C₄), 60.65 (C₂), 68.05 (C₆), 126.89 (C₁₁), 128.05, 129.56 (C₉, C₁₀, C₁₂, C₁₃), 138.24 (C₈), 174.38 (C₃).

### Synthesis of (2S,3R,4S)-ethyl 2-amino-4-hydroxy-3-phenylpentanoate (10c-SRS)

10c (SRS): Obtained from reduction of amino ester with NaBH₄ or NaBH₄/CeCl₃.7H₂O as a clear oil. ¹H NMR (CDCl₃, 200 MHz) δ 0.82 (t, ³J (H₁, H₂)=7.2 Hz, 3H, H₁), 0.91 (d, ³J (H₇, H₆)=6.2 Hz, 3H, H₇), 2.71 (brs, 4H, H₁₄, H₁₅, H₅), 3.76 (m, 1H, H₆), 3.86 (d, ³J (H₄, H₅)=10.0 Hz 1H, H₄), 3.98 (q, ³J (H₂, H₁)=7.1 Hz, 2H, H₂), 7.06-7.33 (m, 5H, H₉, H₁₀, H₁₁, H₁₂, H₁₃).

### Synthesis of (2S,3R,4S)-ethyl 2-amino-4-hydroxy-3-phenylpentanoate (11c-SRR)

11c (SRR): Obtained from reduction of amino ester with NaBH₄ or with Raney nickel, 37%, as a clear oil. ¹H NMR (CDCl₃, 300 MHz) δ: 1.16 (d, ³J (H₅, H₄)=6.5 Hz, 3H, H₅), 3.69 (m, 1H, H₃), 4.09 (d, ³J (H₂, H₃)=8.1 Hz, 1H, H₂), 4.84 (m, 1H, H₄), 7.08-7.39 (m, 5H, H₇, H₈, H₉, H₁₀, H₁₁). ¹³C NMR (CDCl₃, 75 MHz): δ 16.22 (C₅), 51.99, 56.00 (C₂, C₃), 76.75 (C₄), 127.87 (C₉), 128.85, 129.07 (C₇, C₈, C₁₀, C₁₁), 133.20 (C₆), 178.94 (C₁).

### Synthesis of Compound 11d

11d: SSR isomer was obtained as a major product either from one step deprotection-reduction sequence or from reduction of the corresponding amino ester with sodium borohydride, 60%, as a clear oil. The SSS isomer was obtained as a major product from the reduction of the corresponding amino ester with NaBH₄ or NaBH₄/CeCl₃, 75%, as a clear oil.

¹H₁ NMR (CDCl₃, 300 MHz): δ 1.26 (m, 3H, H₁₂), 2.24 (brs, 2H, H₁₃), 2.39-3.11 (m, 3H, H₄, H₃), 3.85 (d, ³J (H₂, H₃)=6.5 Hz, 1H, H₂), 4.14 (m, 1H, H₁₁), 7.19-7.33 (m, 5H, H₆, H₇, H₆, H₉, H₁₀). ¹³C₁ (CDCl₃ 75 MHz): δ 20.34 (C₁₂), 30.65 (C₄), 46.82 (C₃), 55.08 (C₂), 68.22 (C₁₁), 126.11 (C₈), 128.66 (C₆, C₇, C₉, C₁₀), 139.74 (C₅), 174.21 (C₁). ¹H₂NMR (CDCl₃, 300 MHz): δ 1.26 (m, 3H, H₁₂), 2.24 (brs, 2H, H₁₃), 2.39-3.11 (m, 3H, H₄, H₃), 3.89 (d, ³J (H₂, H₃)=7.2 Hz, 1H, H₂), 4.42 (m, 1H, H₁₁), 7.19-7.33 (m, 5H, H₆, H₇, H₈, H₉, H₁₀). ¹³C NMR (CDCl₃, 75 MHz): δ 19.80 (C₁₂), 32.00 (C₄), 47.40 (C₃), 52.56 (C₂), 78.07 (C₁₁), 126.51 (C₆, C₇, C₉, C₁₀), 138.46 (C₅), 178.02 (C₁).

General Procedure for Hydrolysis of Aminolactones and/or γ-hydroxy-α-amino Esters

To a solution of amino lactones and/or γ-hydroxy-α-aminoesters in H₂O/MeOH (0.35 M) was added 1.2 equivalents of LiOH. The reaction mixture was stirred at room temperature for 24 h, followed by additon of 1.2 equivalents of acetic acid. The solvent was removed under reduced pressure and the crude was purified by recrystallization and/or using Dowex.

The following compounds were prepared using the general procedures as described above.

### Synthesis of (2S,3S,4S)-2-amino-4-hydroxy-3-methylhexanoic acid (12b)

12b: 75% as a white solid. ¹H NMR (D₂O, 300 MHz): δ 0.90 (d, ³J (H₇, H₃)=7.1 Hz, 3H, H₇), 0.93 (t, ³J (H₆, H₅)=7.2 Hz, 3H, H₆), 1.56 (m, 2H, H₅), 2.35 (m, 1H, H₃), 3.84 (m, 1H, H₄), 3.88 (d, ³J (H₂, H₃)=2.65 Hz, 1H, H₂). ¹³C NMR (D₂O, 75 MHz): δ 5.77 (C₆), 9.86 (C₇), 27.76 (C₅), 36.74 (C₃), 60.48 (C₂), 77.05 (C₄), 174.51 (C₁). MS (EI) m/z: 132.0675 (M—C₂H₅); 150° C.

### Synthesis of (2S,3S,4R)-2-amino-4-hydroxy-3-methylhexanoic acid (13b)

13b: 75% as a white solid. ¹H NMR (D₂O, 300 MHz): δ 0.96 (t, ³J (H₆, H₅)=7.2 Hz, 3H, H₆), 0.99 (d, ³J (H₇, H₃)=7.1 Hz, 3H, H₇), 1.50-1.67 (m, 2H, H₅, H₅), 2.23 (m, 1H, H₃), 3.56 (m, 1H, H₄), 3.99 (d, ³J (H₂, H₃)=3,01 Hz, 1H, H₂). ¹³C NMR (D₂O, 75 MHz): δ 9.52 (C₆), 11.78 (C₇), 27.48 (C₅), 38.02 (C₃), 56.11 (C₂), 75.38 (C₄), 174.77 (C₁). MS (EI) m/z: 116.1068 (M−CO₂H); 165° C.

### Synthesis of (S)-2-amino-2-((1S,2S)-2-hydroxycyclohexyl)acetic acid (12e)

12e: 60% as a white solid. ¹H NMR (D₂O, 300 MHz): δ 1.24-2.01 (m, 8H, H₅, H₆, H₇, H₈), 2.13 (m, 1H, H₃), 3.84 (d, ³J (H₂, H₃)=3.0 Hz, 1H, H₂), 4.22 (m, 1H, H₄). ¹³C NMR (D₂O, 75 MHz) δ: 19.07, 20.20, 25.27 (C₆, C₇, C₈), 33.27 (C₅), 41.11 (C₃), 59.86 (C₂), 70.69 (C₄), 174.44 (C₁). MS (EI) m/z: 128.1070 (M−CO₂H); 175° C.

### Synthesis of (S)-2-amino-2-((1S,2R)-2-hydroxycyclohexyl)acetic acid (13e)

13e: 60% as a white solid. ¹H NMR (D₂O, 300 MHz): δ 1.19-1.40 (m, 4H), 1.62-1.80 (m, 4H), 1.62-1.80 (m, 3H), 1.85-2.05 (m, 2H), 3.46 (m, 1H, H₄), 3.98 (d, ³J (H₂, H₃)=2.8 Hz, 1H, H₂). ¹³C (D₂O, 75 MHz): δ (ppm) : 24.41, 25.24, 26.44 (C₆, C₇, C₈), 35.49 (C₅, 45.50 (C₃), 56.68 (C₂), 70.94 (C₄), 174.27 (C₁). MS (EI) m/z: 128.1083 (M−CO₂H), 170° C. MS (El) m/z: 174 (M+H)+.

### Synthesis of (S)-2-amino-2-((1S,2S)-2-hydroxycyclohexyl)acetic acid (12f)

12f: 68% as a white solid. ¹H NMR (D₂O, 300 MHz): δ 1.34-1.98 (m, 10H, H₅, H₆, H₇, H₈, H₉), 2.32 (m, 1H, H₃), 3.88 (d, ³J (H₂, H₃)=2.2 Hz, 1H, H₂), 4.26 (m, 1H, H₄). ¹³C NMR (D₂O, 75 MHz): δ 20.89, 21.17, 27.63, 28.63 (C₆, C₇, C₈, C₉), 36.26 (C₇), 43.56 (C₃), 60.67 (C₂), 74.35 (C₄), 174.63 (C₁). MS (EI) m/z: 142.1237 (M−CO₂H); 185° C.

### Synthesis of (S)-2-amino-2-((1S,2R)-2-hydroxycycloheptyl)acetic acid (13f)

13f: 68% as a white solid. ¹H NMR (D₂O, 300 MHz): δ 1.39-1.92 (m, 10H, H₅, H₆, H₇, H₈, H₉), 2.10 (m, 1H, H₃), 3.70 (m, 1H, H₄), 3.99 (d, ³J (H₂, H₃)=2.5 Hz, 1H, H₂). ¹³C NMR (D₂O, 75 MHz): δ 21.43, 25.45, 27.25, 27.69 (C₆, C₇, C₈, C₉), 36.50 (C₅), 47.48 (C₃), 58.31 (C₂), 73.03 (C₄), 174.64 (C₁). MS (EI) m/z: 142.1222 (M−CO₂H); 170° C.

### Synthesis of (2S,3S,4S)-2-amino-4-hydroxy-3-phenylpentanoic acid (12c)

12c: 37% as a white solid. ¹H (D₂O, 300 MHz): δ 1.13 (d, ³J (H₅, H₄)=6.4 Hz, 1H, H₅), 3.20 (dd, ³J (H₃, H₄)=4.9 Hz, ³J (H₃, H₂)=6.5 Hz, 1H, H₃), 4.16 (d, ³J (H₂, H₃)=6.5 Hz, 1H, H₂), 4.43 (m, 1H, H₄), 7.3-7.45 (m, 5H, H₇, H₈, H₉, H₁₀, H₁₁); ¹³C NMR (D₂O, 50 MHz) δ 21.04 (C₅), 52.48 (C₃), 58.54 (C₂), 68.33 (C₄), 128.60 (C₉), 129.35, 130.36 (C₇, C₈, C₁₀, C₁₁), 134.89 (C₆), 173.73 (C₁). MS (EI) m/z: 191.0934 (M−H₂O); 125° C.

### Synthesis of (2S,35,4R)-2-amino-4-hydroxy-3-phenylpentanoic acid (13c)

13c: 37% as a white solid. ¹H NMR (D₂O, 300 MHz): δ 1.19 (d, ³J (H₅, H₄)=6.1 Hz, 3H, H₅), 3.30 (dd, ³J (H₃, H₄)=8.3 Hz, ³J (H₃, H₂)=4.2 Hz, 1H, H₃), 4.27 (d, ³J (H₂, H₃)=4.2 Hz, 1H, H₂), 4.35 (m, 1H, H₄), 7.29-7.45 (m, 5H, H₇, H₈, H₉, H₁₀, H₁₁). ¹³C NMR (D₂O, 75 MHz): δ 21.40 (C₅), 52.92 (C₃), 56.27 (C₂), 67.39 (C₄), 128.50 (C₉), 129.44 (C₇, C₈, C₁₀, C₁₁), 136.14 (C₆), 173.92 (C₁). MS (EI) m/z: 191.0932 (M−H₂0); 160° C.

### Synthesis of a Mixture of (2S,3S,4S)-2-amino-3-benzyl-3-hydroxypentanoic acid (12d) and (2S ³S,4R)-2-amino-3-benzyl-3-hydroxypentanoic acid (13d)

12d & 13d: 60:40 mixture of diastereoisomers, 63% as a white solid. ¹H₁ NMR (D₂O, 300 MHz): δ 1.24 (d, ³J (H₅, H₄)=6.4 Hz, 3H, H₅), 2.29 (m, 1H, H₃), 2.76 (m, 2H, H₆), 3.95 (m, 1H, H₄), 4.08 (d, ³J (H₂, H₃)=1.5 Hz, 1H, H₂), 7.28-7.42 (m, 5H, H₈, H₉, H₁₀, H₁₁, H₁₂). ¹³C₁ NMR (D₂O, 75 MHz): δ 21.17 (C₅), 32.46 (C₆), 46.72 (C₃), 54.95 (C₂), 67.03 (C₄), 126.99 (C₁₀), 129.12, 129.64 (C₈, C₉, C₁₁, C₁₂), 139.64 (C₇), 174.33 (C₁). ¹H₂ NMR (D₂O, 300 MHz): δ 1.16 (d, ³J (H₅, H₄)=6.8 Hz, 3H, H₅), 2.61 (m, 1H, H₃), 2.66-2.97 (m, 2H, H₆), 3.90 (d, ³J (H₂, H₃)=1.9 Hz, 1H, H₂), 4.16 (m, 1H, H₄), 7.31-7.40 (m, 5, H₈, H₉, H₁₀, H₁₁, H₁₂). ¹³C₂ NMR (D₂O, 75 MHz): δ 21.05 (C₅), 29.69 (C₆), 46.22 (C₃), 59.06 (C₂), 70.98 (C₄), 126.99 (C₁₀), 129.02, 129.34 (C₈, C₉, C₁₁, C₁₂), 140.74 (C₇), 173.85 (C₁). MS (EI) m/z: 205.1124 (M−H₂O) 170° C. MS (EI) m/z: 223.1206 (M), 160° C.

### Synthesis of (2S,3R,4S)-2-amino-4-hydroxy-3-methylhexanoic acid (14b)

14b: 75% as a white solid. ¹H NMR (D₂O, 300 MHz): δ 0.96 (m, 6H, H₆, H₇), 1.60 (m, 2H, H₅), 2.01 (m, 1H, H₃), 3.60 (m, 1H, H₄), 3.90 (d, ³J (H₂, H₃)=4.1 Hz, 1H, H₂). ¹³C NMR (D₂O, 75 MHz): δ 9.30 (C₆), 12.59 (C₇), 27.51 (C₅), 39.61 (C₃), 57.27 (C₂), 75.35 (C₄), 174.20 (C₁). MS (EI) m/z: 132.0661 (M−C₂H₅), 140° C.

### Synthesis of (2S,3R,4R)-2-amino-4-hydroxy-3-methylhexanoic acid (15b)

15b: 75% as a white solid. ¹H NMR (D₂O, 300 MHz): δ 0.89 (t, ³J (H₆, H₅)=7.1 Hz, 3H, H₆), 1.06 (d, ³J (H₇, H₃)=7.3 Hz, 3H, H₇), 1.51 (m, 2H, H₅), 2.25 (m, 1H, H₃), 3.73 (m, 1H, H₄), 3.82 (d, ³J (H₂, H₃)=3.2 Hz, 1H, H₂). ¹³C NMR (D₂O, 75 MHz): δ 9.04 (C₆), 9.86 (C₇), 27.60 (C₅), 36.64 (C₃), 60.23 (C₂), 74.37 (C₄), 174.27 (C₁). MS (EI) m/z: 116.1079 (M−CO₂H), 115° C.

### Synthesis of (S)-2-amino-2-((1R,2S)-2-hydroxycyclohexyl)acetic acid (14e)

14e: 60% as a white solid. ¹H NMR (D₂O, 300 MHz): δ 1.05-2.05 (m, 9H, H₅, H₆, H₇, H₈, H₃), 3.65 (m, 1H, H₄), 3.87 (d, ³J (H₂, H₃)=4.9 Hz, 1H, H₂), ¹³C NMR (D₂O, 75 MHz): δ 24.36, 24.98, 26.84 (C₆, C₇, C₈), 35.42 (C₅), 45.88(C₃), 57.65 (C₂), 72.55 (C₄), 173.97 (C₁): MS (EI) m/z: 128.1070 (M−CO₂H), 165° C.

### Synthesis of (S)-2-amino-2-((1 R,2R)-2-hydroxycyclohexyl)acetic acid (15e)

15e: 60% as a white solid. ¹H NMR (D₂O, 300 MHz): δ 1.26-2.11 (m, 9H, H₃, H₅, H₆, H₇, H₈), 3.76 (d, ³J (H₂, H₃)=4.4 Hz, 1H, H₂), 4.12 (m, 1H, H₄). ¹³C NMR (D₂O, 75 MHz): δ 19.36, 23.78, 25.4 (C₆, C₇, C₈), 33.07 (C₅), 40.96 (C₃), 59.35 (C₂), 68.32 (C₄), 174.44 (C₁). MS (EI) m/z: 128.1083 (M−CO₂H); 120° C.

### Synthesis of (S)-2-amino-2-((1R,2S)-2-hydroxycycloheptyl)acetic acid (14f)

14f: 68% as a white solid. ¹H NMR (D₂O, 300 MHz): δ 1.32-1.81 (m, 10H, H₅, H₆, H₇, H₈, H₉), 2.19 (m, 1H, H₃), 3.82 (d, ³J (H₂, H₃)=3.7 Hz, 1H, H₂), ¹³C NMR (D₂O, 75 MHz): δ 21.12, 24.36, 26.94, 27.86 (C₆, C₇, C₈, C₉), 35.98 (C₅), 43.45 (C₃), 60.92 (C₂), 71.54 (C₄), 174.79 (C₁). MS (EI) m/z: 142.1236 (M−CO₂H), 165° C.

### Synthesis of (S)-2-amino-2-((1R,2R)-2-hydroxycycloheptyl)acetic acid (15f)

15f: 68% as a white solid. ¹H NMR (D₂O, 300 MHz): δ 1.32-1.89 (m, 11H, H₃, H₅, H₆, H₇, H₈, H₉), 3.90 (d, ³ J (H₂, H₃)=3.4 Hz, 1H, H₂), 4.05 (m, 1H, H₄). ¹³C NMR (D₂O, 75 MHz): δ 21.89, 24.89, 27.07, 28.27 (C₆, C₇, C₈, C₉), 36.02 (C₅), 48.65 (C₃), 57.68 (C₂), 73.43 (C₄), 174.14 (C,). MS (EI) m/z: 169.1105 (M=H₂O), 160° C.

### Synthesis of (2S,3R,4R)-2-amino-4-hydroxy-3-phenylpentanoic acid (15c)

15c: 37% as a white solid. ¹H NMR (D₂O, 300 MHz): δ 1.31 (d, ³J (H₅, H₄)=6.2 Hz, 3H, H₅), 3.08 (m, 1H, H₃), 4.14 (d, ³J (H₂, H₃)=5.0 Hz, 1H, H₂), 4.53 (m, 1, H₄), 7.37-7.42 (m, 5H, H₇, H₈, H₉, H₁₀, H₁₁). ¹³C NMR (MeOD, 50 MHz): δ 22.13 (C₅), 52.60 (C₃), 52.60 (C₃), 60.98 (C₂), 69.71 (C₄), 128.59 (C₉), 129.64, 131.47 (C₇, C₈, C₁₀, C₁₁), 138.01 (C₆), 173.26 (C₁). MS (EI) m/z: 191.0952 (M−H₂O), 180° C.

### Synthesis of (2S,3R,4S)-2-amino-3-benzyl-3-hydroxypentanoic acid (14d)

14d: 63% as a white solid. ¹H NMR(D₂O, 300 MHz): δ 1.31 (d, ³J (H₅, H₄)=6.4 Hz, 3H, H₅), 2.46 (m, 1H, H₃), 2.66-3.14 (m, 2H, H₆), 3.65 (d, ³J (H₂, H₃)=3 Hz, 1H, H₂), 4.12 (m, 1H, H₄), 7.33-7.43 (m, 5H, H₈, H₉, H₁₀, H₁₁, H₁₂). ¹³C NMR (D₂O, 75 MHz): δ 20.79 (C₅), 30.03 (C₆), 45.77 (C₃), 56.95 (C₂), 68.17 (C₄), 127.16 (C₁₀), 129.39 (C₈, C₉, C₁₁, C₁₂), 139.43 (C₇), 174.38 (C₁). MS (EI) m/z: 223.1206 (M), 225° C.

### Synthesis of (2S,3R,4R)-2-amino-3-benzyl-3-hydroxypentanoic acid (15d)

15d: 63% as a white solid. ¹H NMR (D₂O, 300 MHz): δ 1.26 (d, ³J (H₅, H₄)=6.5 Hz, 3H, H₅), 2.45 (m, 1H, H₃), 2.83 (m, 2H, H₆), 3.86 (d, ³J (H₂, H₃)=2.2 Hz, 1H, H₂), 3.91 (m, 1H, H₄), 7.32-7.44 (m, 5H, H₈, H₉, H₁₀, H₁₁, H₁₂). ¹³C NMR (D₂O, 75 MHz): δ 21.49 (C₅), 34.81 (C₆), 46.87 (C₃), 55.19 (C₂), 67.99 (C₄), 127.14 (C₁₀), 129.25, 129.57 (C₈, C₉, C₁₁, C₁₂), 139.43 (C₇), 174.44 (C₁). MS (EI) m/z: 205.1099 (M−H₂O), 180° C.

### Synthesis of Compound 17

A solution of 4-hydroxyproline methyl ester hydrochloride (16) (10.0 g, 55.3 mmol) and chlorotrimethylsilane (15.0 g, 138.1 mmol) in dichloromethane (200 mL) was stirred at 0° C. To this solution was added triethylamine (19.6 g, 193.4 mmol). The solution was then heated to reflux for 1 h. The mixture was cooled to 0° C., and a solution of methanol (3.3 mL) in dichloromethane (16.5 mL) was added. The reaction mixture was stirred at room temperature for 1 h. To the resulting mixture were added PhF-Br (17.7 g, 55.3 mmol), triethylamine (5.59 g, 55.3 mmol) and Pb(NO₃)₂ (16.5 g, 49.8 mmol). The mixture was stirred at room temperature under nitrogen for 12 h. The mixture was filtered and solvent was evaporated. The residue was redissolved in a solution of citric acid (23 g) in methanol (230 mL). The mixture was stirred at room temperature for 1 h. Solvent was evaporated, and the residue was redissolved in ethyl acetate (300 mL), washed with water (200 mL) and brine. The organic layer was dried with magnesium sulfate and evaporated to obtain crude compound N-PhF-4-hydroxyproline methyl ester (17) (20 g, 94%) with 60% purity. It was used as such without further purification.

### Synthesis of Compound 18

A solution of oxalyl chloride (1.98 g, 15.6 mmol) in dry dichloromethane (45 mL) was stirred at −60° C. under nitrogen. To this solution, was added DMSO (2.0 mL, 27.9 mmol) dropwise over a period of 5 min. The mixture was stirred 15 min. at the same temperature. Then, a solution of N-PhF-4-hydroxyproline methyl ester (17) (4.30 g, 11.15 mmol) in dichloromethane (45 mL) was added dropwise using an addition funnel over a period of 10 min. The reaction mixture was stirred at −60° C. for 45 min. Then, triethylamine (5.97 g, 59.0 mmol) was added to the mixture, and temperature was allowed to reach 0° C. The reaction mixture was poured in an extraction funnel and was washed with water (50 mL). The organic layer was dried with magnesium sulfate and evaporated. The crude product was purified by silica gel chromatography to obtain pure N-PhF-4-oxoproline methyl ester (18) (2.3 g, 54%).

### Synthesis of Compound 19

A solution of N-PhF-4-oxoproline methyl ester (18) (3.00 g, 7.82 mmol) in THF (30 mL) and HMPA (3 mL) was stirred at −55° C. under nitrogen. To this solution was added a 2.5M solution of butyllithium in hexane (3.30 mL, 8.22 mmol). The mixture was stirred at −55° C. for 1 h. Then was added iodomethane (1.46 mL, 23.46 mmol) and the reaction mixture was allowed to reach −10° C. The mixture was stirred at this temperature for 30 min. It was then cooled to −50° C. and a 10% solution of H₃PO₄ (10 mL) was added. The mixture was extracted with ether (2×50 mL). The combined organic phase was washed with brine and dried over magnesium sulfate. The solvent was removed under reduced pressure and the crude product was purified by silica gel chromatography to obtain pure N-PhF-3-methyl-4-oxoproline methyl ester (19) (1.0 g; 30%). 19: ¹H NMR (500 MHz, CDCl₃): δ 7.71 (m, 2H), 7.50 (m, 2H), 7.41-7.37 (m, 4H), 7.28-7.23 (m, 5H), 3.75 (d, 1H); 3.35 (d, 1H), 3.27 (d, 1H), 3.11 (s, 3H), 2.53 (m, 1H), 1.05 (d, 3H).

### Synthesis of Compound 23

A solution of N-PhF-4-oxoproline methyl ester (18) 34 g, 2.17 mmol) in THF (50 mL) and HMPA (15 mL) was stirred at −78° C. under nitrogen. To this solution was added a 0.5M solution of KHMDS in toluene (17.4 mL, 8.70 mmol). The mixture was stirred at −78° C. for 1 h. Then was added iodomethane (1.35 mL, 21.7 mmol) and the reaction mixture was stirred for 12 h. To this mixture was added a 10% aqueous solution of KH₂PO₄. The mixture was extracted with ethyl acetate (2×25 mL). The organic extracts were collected, washed with brine and dried with sodium sulfate, concentrated under reduced pressure. The crude compound was dissolved in hexane:ethyl acetate (3:1) and filtered on silica gel to obtain pure N-PhF-3,3-dimethyl-4-oxoproline methyl ester (23) (0.63 g. 70%). 23: ¹H NMR (500 MHz, CDCl₃): δ 7.74 (d, 1H), 7.67 (d, 1H), 7.43-7.25 (m, 11H), 3.97 (d, 1H), 3.75 (d, 1H), 3.43 (s,1H), 2.95 (s, 3H), 1.37 (s, 3H), 0.84 (s, 3H).

### Synthesis of Compound 27

A solution of N-PhF-4-oxoproline methyl ester (18) (1.30 g, 3.39 mmol) in THF (10 mL) and HMPA (15 mL) was stirred at −78° C. under nitrogen. To this solution was added a 1.0 M solution of LiHMDS in THF (8.80 mL, 8.80 mmol). The mixture was stirred at −78° C. for 1 h. Acetaldehyde (1.75 eq) was added, and the reaction mixture was allowed to reach −55° C. After stirring for 3 h, 10% aqueous solution of H₃PO₄ (5 mL) was added. The mixture was extracted with ether (2×25 mL). The organic extracts were collected, washed with brine and dried with sodium sulfate, and concentrated under reduced pressure. The crude compound was purified by silica gel chromatography to afford pure N-PhF-3-(2-hydroxy-ethyl)-4-oxoproline methyl ester (27). ¹H NMR was in accord with the structure.

### Synthesis of Compound 28

A solution of N-PhF-4-oxoproline methyl ester (18) (1.30 g, 3.39 mmol) in THF (10 mL) and HMPA (15 mL) was stirred at −78° C. under nitrogen. To this solution was added a 1.0 M solution of LiHMDS in THF (8.80 mL, 8.80 mmol). The mixture was stirred at −78° C. for 1 h. Then was added benzaldehyde (600 μL, 5.93 mmol, 1.75 eq.) and the reaction mixture was allowed to reach −55° C. After stirring for 3 h, 10% aqueous solution of H₃PO₄ (5 mL) was added. The mixture was extracted with ether (2×25 mL). The organic extracts were collected, washed with brine and dried with sodium sulfate, and concentrated under reduced pressure. The crude compound was purified by silica gel chromatography to afford pure N-PhF-3-hydroxyphenylmethyl-4-oxoproline methyl ester (28) (0.98 g, 60%). ¹H NMR was in accord with the structure.

### Synthesis of Compound 20

A solution of N-PhF-3-methyl-4-oxoproline methyl ester (19) (1.00 g, 2.52 mmol) in THF/methanol (1:1) (20 mL) was stirred at −78° C. To this solution was added a solution of sodium borohydride (0.238 g, 6.29 mmol) in methanol (5 mL). The mixture was stirred for 5 days and reaction was still not complete. The mixture was allowed to reach −10° C. and was stirred for 2 h. LC-MS analysis showed the presence of two compounds of same molecular weight but with different retention time i.e. two diastereoisomers. The reaction mixture was cooled at −70° C. and a 10% aqueous H₃PO₄ solution (10 mL) was added. After concentrating the mixture under reduced pressure, the resulting mixture was extracted with ethyl acetate (2×25 mL). The organic extracts were collected, washed with brine and dried with sodium sulfate, and concentrated. The crude compound was purified by silica gel chromatography to afford pure N-PhF-3-methyl-4-hydroxy-proline methyl ester (20) (0.485 g; 49%). 20: ¹H NMR (500 MHz, CDCl₃): δ 7.74 (d, 1H), 7.67 (d, 1H), 7.43-7.25 (m, 11H), 3.97 (d, 1H), 3.75 (d, 1H), 3.43 (s, 1H), 2.95 (s, 3H), 1.37 (s, 3H), 0.84 (s, 3H).

### Synthesis of Compound 24

A solution of N-PhF-3,3-dimethyl-4-oxoproline methyl ester (23) (0.860 g, 2.09 mmol) in THF/methanol (1:1) (12 mL) was stirred at −78° C. To this solution was added sodium borohydride (0.158 g, 4.18 mmol). The mixture was allowed to reach −10° C. and was stirred for 3 h, and then cooled at −70° C. and a 10% aqueous H₃PO₄ solution (10 mL) was added. After concentrating the reaction mixture under reduced pressure, the resulting mixture was extracted with ethyl acetate (2×25 mL). The organic extracts were collected, washed with brine and dried with sodium sulfate, and concentrated. The crude compound was purified by silica gel chromatography to afford pure N-PhF-3,3-dimethyl-4-hydroxyproline methyl ester (24) (600 mg, 69%). 24: ¹H NMR (500 MHz, CDCl₃): δ 7.75 (d, 1H), 7.60 (m, 3H), 7.54 (d, 1H), 7.44 (t, 1H), 7.30-7.21 (m, 6H), 7.08 (t, 1H), 4.14 (t, 1H), 3.58 (t, 1H), 3.33 (s, 3H), 2.95 (t, 1H), 2.69 (s, 1H), 0.79 (s, 3H), 0.50 (s, 3H).

### Synthesis of Compound 29

A solution of N-PhF-3-hydroxyphenylmethyl-4-oxoproline methyl ester (27) in THF/methanol (1:1) (20 mL) was stirred at −78° C. To this solution was added sodium borohydride (2.5 eq), and the mixture was stirred for 12 h before allowing the temperature to reach −10° C. 10% aqueous H₃PO₄ solution (10 mL) was added, and the mixture was concentrated under reduced pressure. The resulting mixture was extracted with ethyl acetate (2×25 mL). The organic extracts were collected, washed with brine and dried with sodium sulfate, and concentrated. The crude compound was purified by silica gel chromatography to afford N-PhF-3-(2-hydroxy-ethyl)-4-hydroxy-proline methyl ester (29) as an oil (1.3 g). The product was used for further reaction without any purification.

### Synthesis of Compound 30

A solution of N-PhF-3-hydroxyphenylmethyl-4-oxoproline methyl ester (28) (0.980 g, 1.97 mmol) in THF/methanol (1:1) (20 mL) was stirred at −78° C. To this solution was added sodium borohydride (0.187 g, 4.92 mmol). The mixture was stirred for 12 h and then was allowed to reach −10° C. LC-MS analysis showed a complete reaction, therefore 10% aqueous H₃PO₄ solution (10 mL) was added. The reaction mixture was concentrated under reduced pressure, and the resulting mixture was extracted with ethyl acetate (2×25 mL). The organic extracts were collected, washed with brine and dried with sodium sulfate, and concentrated to obtain pure N-PhF-3-hydroxyphenylmethyl-4-hydroxy-proline methyl ester (30) as an oil (1.3 g, with 85% purity). The product was used as such for next reaction without any further purification.

### Synthesis of Compound 21

A solution of N-PhF-3-methyl-4-hydroxyproline methyl ester (20) (0.485 g, 1.21 mmol) in ethanol (7 mL) was stirred at room temperature. To this solution was added a 4N NaOH (6 mL, 24.3 mmol) solution and the mixture was heated to reflux for 5 days. The reaction mixture was neutralized with a 10% aqueous solution of KH₂PO₄ after LC-MS analysis showed no sign of the presence of the starting material. The mixture was extracted with ethyl acetate (2×25 mL). The organic extracts were collected, washed with brine and dried with sodium sulfate, and concentrated under reduced pressure. The crude product was purified by trituration with ethyl acetate/hexane, to afford N-PhF-3-methyl-4-hydroxyproline (21) (0.290 g; 62%) with a HPLC purity of 95% purity.

### Synthesis of Compound 25

A solution of N-PhF-3,3-dimethyl-4-hydroxyproline methyl ester (24) (0.595 g, 1.44 mmol) in THF (40 mL) was stirred in a Parr reactor at room temperature. To this solution was added (Boc)₂0 (0.690 g, 3.17 mmol) and 10% palladium on carbon (200 mg). The reactor was sealed and hydrogen was added (75 psi). The mixture was stirred at room temperature for 12 h. After the reaction was complete, the mixture was filtered and evaporated. The crude compound was triturated with hexane and dried to afford Boc intermediate (25).

### Synthesis of Compound 26

The BOC intermediate (25) (0.163 g, 0.597 mmol) was dissolved in dioxane (3 mL) and concentrated HCl (3 mL) was added. The mixture was stirred at 60° C. for 4 days. At this stage, LC-MS showed the completion of the reaction. The white precipitates formed during the reaction were filtered off and the filtrate was concentrated under reduced pressure and water was removed using a freeze-dryer to afford 26.

### Synthesis of Compound 31

A solution of 860 mg N-PhF-3-(2-hydroxy-ethyl)-4-hydroxyproline methyl ester (29) (2 mmol) in ethanol (10 mL) was stirred at room temperature. To this solution was added a 2N aqueous solution of NaOH (1.5 ml, 3.00 mmol) and the mixture was stirred at room temperature for 5 h. More NaOH pellets (0.100 g, 2.50 mmol) were added. The reaction mixture was stirred at room temperature for another 24 h. As HPLC revealed 25% conversion, 2N aqueous solution of KOH (1.0 mL, 2.0 mmol) was added, and the mixture was stirred for 6 days. The reaction mixture was concentrated under reduced pressure, and the residue was redissolved in ethyl acetate (25 mL). The mixture was washed with HCl (0.5N). The organic layer was washed with brine and dried with sodium sulfate, and concentrated. The crude compound was purified by silica gel chromatography to afford pure N-PhF-3-(2-hydroxy-ethyl)-4-hydroxyproline (31) (400 mg, 48%).

### Synthesis of Compound 32

To a solution of N-PhF-3-hydroxyphenylmethyl-4-hydroxyproline methyl ester (30) (0.968 g, 1.97 mmol) in ethanol (10 mL), at room temperature, was added 2N aqueous solution of NaOH (1.5 ml, 3 mmol) and the mixture was stirred for 5 h. As little progress was observed by HPLC, more NaOH(s) (0.100 g, 2.50 mmol) was added and the reaction mixture was stirred at room temperature for another 24 h. At this stage, 25% hydrolysis was observed (HPLC), therefore, 2N aqueous solution of KOH (1.0 mL, 2.0 mmol) was added and the mixture was stirred for 6 more days. The reaction mixture was concentrated under reduced pressure and the residue was dissolved in ethyl acetate (25 mL). The mixture was washed with HCl (0.5N), followed by washing of the organic layer with brine and drying with sodium sulfate. The reaction mixture was concentrated and the crude product was purified by silica gel chromatography to afford pure N-PhF-3-hydroxyphenylmethyl-4-hydroxyproline (32) (400 mg g, 43%).

### Synthesis of Compound 22

A solution of N-PhF-3-methyl-4-hydroxyproline (21) (0.290 g, 0.752 mmol) in ethanol (45 mL) and acetic acid (5 mL) was stirred in a Parr reactor at room temperature. To this solution was added 10% palladium on carbon (0.400 g). The reactor was sealed and hydrogen was added (100 Psi). The mixture was stirred for 2 h. After completion, the catalyst was filtered off and solvent was removed under reduced pressure. Water was added (20 mL) to the reaction mixture, and the mixture was washed with ether (2×25 mL). Water/acetic acid was removed using 3 lyophilization procedures to obtain compound 22.

### Synthesis of Compound 33

A solution of N-PhF-3-hydroxyethyl-4-hydroxyproline (31) (0.300 g, 0.722 mmol) in ethanol (45 mL) and acetic acid (5 mL) was stirred in a Parr reactor at room temperature. To this solution was added 10% palladium on carbon (0.100 g). The reactor was sealed and hydrogen was added (100 Psi). The mixture was stirred for 1 h. After completion, the mixture was filtered and concentrated under reduced pressure. Water was added (20 mL) to the reaction mixture and the mixture was washed with ether (2×25 mL). Water/acetic acid mixture was removed using lyophilization cycles to afford compound 33.

### Synthesis of Compound 34

A solution of N-PhF-3-hydroxyphenylmethyl-4-hydroxyproline (32) (0.420 g, 0.880 mmol) in ethanol (45 mL) and acetic acid (5 mL) was stirred in a Parr reactor at room temperature. To this solution was added 10% palladium on carbon (0.100 g). The reactor was sealed and hydrogen was added (100 Psi). The mixture was stirred for 1 h. After completion, the mixture was filtered and concentrated under reduced pressure. Water was added (20 mL) to the reaction mixture and the mixture was washed with ether (2×25 mL). Water/acetic acid mixture was removed by lyophilization cycles to afford compound 34.

### Synthesis of Compound 35

Boc-proline methyl ester (10 g, 43.67 mmol) was dissolved in anhydrous tetrahydrofuran (100 mL). The solution was cooled to −78° C. To the cooled solution was added 2M LDA solution (52.4 mmol, 26.2 mL). The enolization reaction was stirred for 45 min. at −78° C., followed by addition of 1.2 equivalents of allyl bromide. The alkylation was allowed to proceed overnight at −78° C. The reaction mixture was then allowed to warm to −20° C. The reaction was finally quenched by adding saturated ammonium chloride solution (100 mL) followed by addition of ethyl acetate (100 mL), and the two layers were separated. The organic layer was washed with brine, dried over magnesium sulfate, and concentrated under reduced pressure to give a yellow oil. The crude product was purified by silica gel column chromatography to obtain pure 35 (6 g).

### Synthesis of Compound 36

To a solution of compound 35 in ethanol (30 mL) was added 2 equivalent of 4N KOH aqueous solution, and the mixture was stirred for 48 h. The reaction mixture was concentrated under reduced pressure, followed by addition of water (50 mL). The basic solution was acidified using HCl 2N to adjust the pH to 3. This was followed by the extraction of the reaction mixture with ethyl acetate (100 mL). The concentration of the organic phase and subsequent recrystallization from ethyl acetate/hexane mixture gave pure Boc-α-allylproline (36) (2.5 g).

### Synthesis of Boc-α-oxiranylmethylproline (37)

Boc-α-allylproline (36) (2 g) was dissolved in methylene chloride (40 mL) and THF (10 mL). m-Chloroperbenzoic acid (2 g) was added and the reaction was stirred for 24 h. The crude reaction mixture was concentrated and extracted with EtOAc/saturated bicarbonate solution. The crude epoxidized allyiproline was purified by silica gel column chromatography to afford pure Boc-α-oxiranylmethylproline (37) (1.1 g).

### Synthesis of α-oxiranylmethyl-proline (38)

Above obtained Boc-α-oxiranylmethylproline (37) was dissolved in methylene chloride (5 mL), to this was added trifluoroacetic acid (5 mL), and the reaction mixture was stirred overnight. The reaction mixture was concentrated under reduced pressure, followed by addition of methylene chloride and concentration of the mixture again. This was repeated three times, followed by addition of water (30 mL) and freeze-drying, twice, to yield pure α-oxiranylmethyl-proline (38) (680 mg). 38: MS: M+H⁺=172.

### Synthesis of Compound 39

To a solution of L-proline methylester hydrochloride (5 g, 30 mmol) in water (20 mL) was added an excess of propylene oxide (20 mL). An exothermic reaction was observed, and the mixture was stirred overnight. After concentrating the reaction mixture under reduced pressure, the crude product was purified by reverse-phase chromatography to give 39 (2.3 g, 42%). 39: MS: M+H⁺=188.

### Synthesis of Compound 40

Above described methyl ester (39) was hydrolyzed in ethanol with 2 equivalents of 2N aqueous KOH and stirring for 48 h. The reaction mixture was neutralized using HCl 0.5 N, before freeze-drying. So obtained crude was purified by reverse-phase-chromatography to obtain 40 (1.15 g, 52%) as a clear oil. 40: MS: M+H⁺=174.

### Synthesis of cyclohexanecarboxylic acid methoxy-methyl-amide (41)

A solution of cyclohexylcarboxylic acid (6.30 g, 49.1 mmol) in acetonitrile (30 mL) was stirred at room temperature. To this solution was added N,N-diisopropylethylamine (DIEA) (12.7 g, 98.3 mmol) and TBTU (16.6 g, 51.6 mmol). The mixture was stirred for 10 min. Then, a solution of N,O-dimethylhydroxylamine hydrochloride (5.75 g, 59.0 mmol) and DIEA (6.35 g, 49.1 mmol) in acetonitrile (30 mL) was added. The mixture was stirred at room temperature for 24 h. The reaction mixture was concentrated under reduced pressure and the crude mixture was redissolved in ethyl acetate (250 mL), and washed with 0.5N NaOH (2×100 mL), 0.5N HCl (2×100 mL) and brine. The organic layer was dried with magnesium sulfate and concentrated. The resulting oil was redissolved in hexane/ethyl acetate (3:1) and filtered through silica gel. The mixture was concentrated to afford 41 (7.4 g, 88%). 41: ¹H NMR (500 MHz, CDCl₃): ε ¹H NMR (CDCl₃): 3.68 (s, 3H), 3.16 (s, 3H), 2.67 (m, 1H), 1.81-1.23 (m, 10H)

### Synthesis of cyclopentanecarboxylic acid methoxy-methyl-amide (42)

To a stirred solution of cyclopentylcarboxylic acid (6.00 g, 52.6 mmol) in acetonitrile (30 mL), at room temperature, was added DIEA (13.6 g, 105.1 mmol) and TBTU (17.7 g, 55.2 mmol), and the mixture was stirred for 10 min. Then, a solution of N,O-dimethylhydroxylamine hydrochloride (6.15 g, 63.1 mmol) and DIEA (6.79 g, 52.6 mmol) in acetonitrile (30 mL) was added. The reaction mixture was stirred at room temperature for 24 h. The reaction mixture was concentrated under reduced pressure and the crude product was redissolved in ethyl acetate (250 mL) and washed with 0.5N NaOH (2×100 mL), 0.5N HCl (2×100 mL) and brine. The organic phase was dried with magnesium sulfate and concentrated. The resulting oil was redissolved in hexane/ethyl acetate (3:1) and filtered through silica gel. After removal of solvent, pure cyclopentanecarboxylic acid methoxy-methyl-amide (42) (8 g, 97%) was obtained.

### Synthesis of 1-cyclohexyl-ethanone (43)

A solution of of cyclohexanecarboxylic acid methoxy-methyl-amide (41) (4.1 g, 23.9 mmol) in dry THF (45 mL) was stirred at −78° C. under nitrogen. To this solution was added a 1.6M solution of methyllithium in THF (15 mL, 23.9 mmol). The reaction mixture was allowed to warm to 0° C., and the mixture was stirred for additional 1 h. A 0.5M solution of HCl (40 mL) was added and the mixture was extracted with ethyl acetate (2×50 mL). The organic extracts were combined, dried with magnesium sulfate and concentrated under reduced pressure to affored 1-cyclohexyl-ethanone (43) (2.83 g, 94%) as a colorless oil. 43: ¹H NMR (500 MHz, CDCl₃): δ 2.33 (m, 1H), 2.13 (s, 3H), 1.88-1.66 (m, 5H), 1.37-1.16 (m, 5H).

### Synthesis of 1-cyclopentyl-ethanone (44)

A solution of cyclopentanecarboxylic acid methoxy-methyl-amide (42) (6.20 g, 39.44 mmol) in dry THF (60 mL) was stirred at −78° C. under nitrogen. To this solution was added a 1.6M solution of methyllithium in THF (24.6 mL, 39.44 mmol). The temperature of the reaction mixture was allowed to reach 0° C., and the mixture was stirred for 1 h. A 0.5M solution of HCl (20 mL) was added and the mixture was extracted with ethyl acetate (2×50 mL). The organic extracts were combined, dried with magnesium sulfate and evaporated to obtain 1-cyclopentyl-ethanone (44) (3.40 g, 77%) as a colorless oil. 44: ¹H NMR (500 MHz, CDCl₃): δ 2.86 (m, 1H), 2.16 (s, 3H), 1.84-1.57 (m, 8H).

### Synthesis of 4-cyclohexyl-2-hydroxy-4-oxo-but-2-enoic acid ethyl ester (47)

A solution of sodium ethoxide was prepared by dissolving sodium (1.00 g, 43.7 mmol) in dry ethanol (100 mL). To this solution, was added cyclohexylmethylketone (43) (4.60 g, 36.4 mmol) and diethyl oxalate (5.33 g, 36.4 mmol). The mixture was stirred for 2 h at room temperature. After removal of the solvent, water (25 mL) and ice (14 g) were added. The mixture was treated with concentrated HCl (7 mL) and then extracted with ethyl acetate (2×100 mL). The organic extracts were combined, washed with brine and dried with sodium sulfate. The crude product obtained after concentrating the reaction mixture under reduced pressure was redissolved in hexane/ethyl acetate (3:1) and filtered through a plug of silica gel. The removal of solvent, afforded 4-cyclohexyl-2-hydroxy-4-oxo-but-2-enoic acid ethyl ester (47) (5.2 g, 63%) as an orange oil. 47: ¹H NMR (500 MHz, CDCl₃): δ 6.39 (s, 1H), 4.35 (q, 2H), 2.37 (m, 1H), 1.91-1.69 (m, 5H), 1.42-1.24 (m, 8H).

### Synthesis of 4-cyclopentyl-2-hydroxy-4-oxo-but-2-enoic acid ethyl ester (48)

A solution of sodium ethoxide was prepared by dissolving sodium (0.84 g, 36.4 mmol) in dry ethanol (80 mL). To this solution was added cyclopentylmethylketone (44) (3.40 g, 30.3 mmol) and diethyl oxalate (4.43 g, 30.3 mmol). The mixture was stirred for 12 h at room temperature. After removal of the solvent, water (15 mL) and ice (10 g) were added. The mixture was treated with concentrated HCl (5 mL) and then extracted with ethyl acetate (2×50 mL). The organic extracts were combined, washed with brine and dried with sodium sulfate. After removal of the solvent, the crude product was redissolved in hexane/ethyl acetate (3:1) and filtered through silica gel. The removal of solvent gave 4-cyclopentyl-2-hydroxy-4-oxo-but-2-enoic acid ethyl ester (48) (3.7 g, 58%) as an orange oil. 48: ¹H NMR (500 MHz, CDCl₃): δ 6.39 (s, 1H), 4.35 (q, 2H), 2.89 (m,1H), 1.82-1.64 (m, 8H), 1.36 (t, 3H).

### Synthesis of 2-hydroxy-4-oxo-4-phenyl-but-2-enoic acid ethyl ester (49)

A solution of sodium ethoxide was prepared by dissolving sodium (4.59 g, 200 mmol) in dry ethanol (450 mL). To this solution was added acetophenone (45) (20.0 g, 166.4 mmol) and diethyl oxalate (24.3 g, 166.4 mmol). The mixture was stirred for 12 h at room temperature. After removal of the solvent, water (80 mL) and ice (60 g) was added. The mixture was treated with concentrated HCl (25 mL), and extracted with ethyl acetate (2×200 mL). The organic extracts were combined, washed with brine and dried with sodium sulfate. The crude product obtained after removal of the solvent was redissolved in hexane/ethyl acetate (3:1) and filtered through silica gel. After removal of the solvent under reduced pressure 2-hydroxy-4-oxo-4-phenyl-but-2-enoic acid ethyl ester (49) (22 g, 60%) was obtained as an orange oil. 49: ¹H NMR (500 MHz, CDCl₃): δ 8.00 (d, 2H), 7.61 (t, 1H), 7.51 (t, 2H), 7.08 (s, 1H), 4.40 (q, 2H), 1.42 (t, 3H).

### Synthesis of 2-hydroxy-5,5-dimethyl-4-oxo-hex-2-enoic acid ethyl ester (50)

A solution of sodium ethoxide was prepared by dissolving sodium (2.75 g. 120 mmol) in dry ethanol (250 mL). To this solution was added pinacolone (46) (10.0 g, 99.8 mmol) and diethyl oxalate (14.6 g, 99.8 mmol). The mixture was stirred for 12 h at room temperature. After removal of the solvent, water (50 mL) and ice (25 g) was added. The mixture was treated with concentrated HCl (7 mL) and extracted with ethyl acetate (2×150 mL). The organic extracts were combined, washed with brine and dried with sodium sulfate. The crude product obtained after removal of the solvent was redissolved in hexane/ethyl acetate (3:1) and filtered through silica gel. After removal of the solvent under reduced pressure, 2-hydroxy-5,5-dimethyl-4-oxo-hex-2-enoic acid ethyl ester (50) was obtained as a colorless oil (22 g, 60%). 50: ¹H NMR (500 MHz, CDCl₃): δ 6.54 (s, 1H), 4.35 (q, 2H), 1.38 (t, 3H), 1.22 (s, 9H).

### Synthesis of 5-cyclohexyl-isoxazole-3-carboxylic acid ethyl ester (51)

A solution of the above depicted enone (47) (5.10 g, 22.4 mmol) in anhydrous ethanol/THF (1:1) (60 mL) was stirred at room temperature. To this solution was added hydroxylamine hydrochloride (1.72 g, 24.7 mmol) and the resulting mixture was stirred 12 h under nitrogen. The mixture was then heated to reflux with a soxhlet filled with molecular sieves for 2 h. After cooling the reaction mixture, solvent was removed under reduced pressure. Water (100 mL) was added and the mixture was extracted with dichloromethane (2×100 mL). The organic extracts were collected and dried with sodium sulfate. After removal of the solvent, the crude product was purified by silica gel chromatography to affored 5-cyclohexyl-isoxazole-3-carboxylic acid ethyl ester (51) as a colorless oil (2.8 g, 56%). 51: ¹H NMR (500 MHz, CDCl₃): δ 6.37 (s, 1H), 4.42 (q, 2H), 2.83 (m, 1H), 2.06 (m, 2H), 1.81 (m, 2H), 1.75 (m, 1H), 1.48-1.26 (m, 8H).

### Synthesis of 5-cyclopentyl-isoxazole-3-carboxylic acid ethyl ester (52)

A solution of the cyclopentyl-enone (48) (3.70 g, 17.4 mmol) in anhydrous ethanol/THF (1:1) (50 mL) was stirred at room temperature. To this solution was added hydroxylamine hydrochloride (1.33 g, 19.1 mmol) and the resulting mixture was stirred 12 h under nitrogen. The mixture was then heated to reflux with a soxlet filled with molecular sieves during 2 h. After cooling the reaction mixture, solvent was evaporated under reduced pressure. Water (50 mL) was added and the mixture was extracted with dichloromethane (2×50 mL). The organic extracts were combined, dried with sodium sulfate, and concentrated. The crude product was purified by silica gel chromatography to give 5-cyclopentyl-isoxazole-3-carboxylic acid ethyl ester (52) as a colorless oil (2 g, 55%). 52: ¹H NMR (500 MHz, CDCl₃): δ 6.38 (s, 1H), 4.42 (q, 2H), 3.25 (m, 1H), 2.11 (m, 2H), 1.80-1.69 (m, 6H), 1.41 (t, 3H).

### Synthesis of 5-phenyl-isoxazole-3-carboxylic acid ethyl ester (53)

A solution of the phenyl-enone (49) (5.00 g, 22.7 mmol) in anhydrous ethanol/THF (1:1) (60 mL) was stirred at room temperature. To this solution was added hydroxylamine hydrochloride (1.73 g, 25.0 mmol) and the resulting mixture was stirred 12 h under nitrogen. The mixture was then heated to reflux with a soxlet filled with molecular sieves during 2 h. The mixture was allowed to cool down and the solvent was evaporated. Water (100 mL) was added and the mixture was extracted with dichloromethane (2×100 mL). The organic extracts were combined, dried with sodium sulfate, and concentrated. The crude product was purified by silica gel chromatography to give 5-phenyl-isoxazole-3-carboxylic acid ethyl ester (53) as a colorless oil (3.89 g, 79%). 53: ¹H NMR (500 MHz, CDCl₃): δ 7.80 (d, 2H), 7.50 (m, 3H), 6.93 (s,1H), 4.47 (q, 2H), 1.44 (t, 3H).

### Synthesis of 5-tert-butyl-isoxazole-3-carboxylic acid ethyl ester (54)

A solution of tert-butyl-enone (50) (6.00 g, 30.0 mmol) in anhydrous ethanol/THF (1:1) (70 mL) was stirred at room temperature. To this solution was added hydroxylamine hydrochloride (2.29 g, 33.0 mmol) and the resulting mixture was stirred 12 h under nitrogen. The mixture was then heated to reflux with a soxlet filled with molecular sieves during 2 h. The mixture was allowed to cool down and the solvent was evaporated. Water (100 mL) was added and the mixture was extracted with dichloromethane (2×100 mL). The organic extracts were combined, dried with sodium sulfate, and concentrated. The crude product was purified by silica gel chromatography to give 5-tert-butyl-isoxazole-3-carboxylic acid ethyl ester (54) as a colorless oil (3 g, 51%). 54: ¹H NMR (500 MHz, CDCl₃): δ 6.37 (s, 1H), 4.43 (q, 2H), 1.41 (t, 3H), 1.37 (s, 9H).

### Synthesis of 5-cyclohexyl-isoxazole-3-carboxylic acid (55)

A solution of cyclohexyl isoxazole ethyl ester (51) (2.80 g, 12.5 mmol) in ethanol (30 mL) was stirred at room temperature. To this solution was added a 2M NaOH solution (9.4 mL, 18.8 mmol). Within a few minutes, precipitates were formed and reaction mixture became a thick paste. TLC showed that the reaction was complete. To the reaction mixture was added 0.5M HCl to adjust pH to 3-4, and then the mixture was extracted with ethyl acetate (2×100 mL). The organic extracts were combined, washed with brine, dried over sodium sulfate, and concentrated to afford 5-cyclohexyl-isoxazole-3-carboxylic acid (55) as white crystals (2.2 g. 90%). 55: ¹H NMR (500 MHz, CDCl₃): δ 9.60 (broad, 1H), 6.44 (s, 1H), 2.86 (m,1H), 2.08 (m, 2H), 1.83 (m, 2H), 1.74 (m, 1H), 1.50-1.28 (m, 5H).

### Synthesis of 5-cyclopentyl-isoxazole-3-carboxylic acid (56)

A solution of cyclopentyl isoxazole ethyl ester (52) (2.00 g, 9.56 mmol) in ethanol (30 mL) was stirred at room temperature. To this solution was added a 2M NaOH solution (7.2 mL 14.4 mmol). After 5 min., TLC showed that the reaction was complete. To the reaction mixture was added 0.5M HCl to adjust the pH to 3-4, followed by extraction with ethyl acetate (2×75 mL). The organic extracts were combined, washed with brine, dried over sodium sulfate, and concentrated to afford 5-cyclopentyl-isoxazole-3-carboxylic acid (56) as white crystals (1.6 g, 92%). 56: ¹H NMR (500 MHz, CDCl₃): δ 9.75 (broad, 1H), 6.45 (s, 1H) 3.26 (m, 1H), 2.13 (m, 2H), 1.80-1.70 (m, 6H).

### Synthesis of 5-phenyl-isoxazole-3-carboxylic acid (57)

A solution of phenyl-substituted isoxazole ethyl ester (53) (1.89 g, 8.70 mmol) in ethanol (30 mL) was stirred at room temperature. To this solution was added a 2M NaOH solution (6.5 mL, 13.1 mmol). After 5 min. TLC showed that the reaction was complete. To the reaction mixture was added 0.5M HCl to adjust the pH to 3-4, before extracting with ethyl acetate (2×75 mL). The organic extracts were combined, washed with brine, dried over sodium sulfate, and concentrated to afford 5-phenyl-isoxazole-3-carboxylic acid (57) was obtained as a white solid (1.54 g, 94%). 57: ¹H NMR (500 MHz, CDCl₃): δ 9.4 (broad, 1H), 7.83 (d, 2H), 7.51 (m, 3H), 6.99 (s, 1H)

### Synthesis of 5-tert-butyl-isoxazole-3-carboxylic acid (58)

A solution of tert-butyl-substituted isoxazole ethyl ester (54) (2.97 g, 15.1 mmol) in ethanol (30 mL) was stirred at room temperature. To this solution was added a 2M NaOH solution (11.3 mL, 22.6 mmol). After 5 min., TLC showed a complete reaction. To the reaction mixture was added 0.5M HCl to adjust the pH to 3-4 before extracting with ethyl acetate (2×75 mL). The organic extracts were combined, washed with brine, dried over sodium sulfate, and concentrated to afford 5-tert-butyl-isoxazole-3-carboxylic acid (58) as a colorless oil (1.54 g; 94%). 58: ¹H NMR (500 MHz, CDCl₃): δ 6.44 (s, 1H), 1.39 (s, 9H).

### Synthesis of 2-amino-4-cyclohexyl-4-hydroxy-butyric acid (59)

A solution of the above depicted cyclohexyl-substituted isoxazole carboxylic acid (55) (2.20 g, 11.3 mmol) in ethanol/water (1:1) (80 mL) was stirred in a Parr reactor at room temperature. To this solution was added a suspension of Raney-Ni (2 g) (pre-washed 5 times with ethanol/water (1:1)) in ethanol/water. The reactor was sealed and hydrogen was added (120 psi). The mixture was stirred at room temperature for 3 h. LC-MS analysis revealed that reaction was not complete. The mixture was stirred for another 12 h, at this stage, LC-MS revealed that the starting material was entirely consumed, yet the major compound was a species with one non hydrogenated double bond. The mixture was filtered and the catalyst was rinsed with ethanol and water. To the filtrate was added 10% palladium on carbon (0.6 g) and acetic acid (10 mL). The reactor was sealed and hydrogen was added (120 psi). The mixture was stirred for 12 h at room temperature. This was followed by heating of the mixture at 50° C. for 4 days with 180 psi pressure of hydrogen. The mixture was filtered and filtrate was concentrated under reduced pressure, and water was removed by lyophilization. So obtained greenish solid of 2-Amino-4-cyclohexyl-4-hydroxy-butyric acid (59) was further purified by reverse-phase chromatography (100% water). The pure fractions were identified by LCMS, collected and lyophilized. 59: MS: M+H⁺=202.

### Synthesis of 2-amino-4-cyclopentyl-4-hydroxy-butyric acid (60)

The procedure described above for compound 59 was followed to synthesize 60 using cyclopentyl-substituted isoxazole carboxylic acid (56) (1.48 g, 8.17 mmol) in ethanol/water (1:1) (60 mL), Raney-Ni (1.5 g), 10% palladium on carbon (0.6 g), acetic acid (10 mL) and heating at 50° C. for 4 days with 180 psi of hydrogen. The purification was carried out using reverse-phase-chromatography. The pure fractions were identified by LCMS, collected and lyophilized. 60: MS: M+H⁺=187.

### Synthesis of 2-amino-4-hydroxy-4-phenyl-butyric acid (61)

The procedure described above for compounds 59 & 60 was followed to synthesize 61 using: phenyl-substituted isoxazole carboxylic acid (57) (0.800 g, 4.23 mmol) in ethanol/water (1:1) (40 mL), Raney-Ni (1 g),10% palladium on carbon (0.6 g), acetic acid (10 mL), and heating at 50° C. for 4 days with 180 psi of hydrogen. The purification was carried out using reverse-phase -chromatography. The pure fractions were identified by LCMS, collected and lyophilized.

### Synthesis of 2-amino-4-hydroxy-5,5-dimethyl-hexanoic acid (62)

The procedure described above for compounds 59, 60 & 61 was followed to synthesize 2-Amino-4-hydroxy-5,5-dimethyl-hexanoic acid (62) using: tert-butyl-substituted isoxazole (58) (2.0 g, 11.8 mmol) in ethanol/water (1:1) (40 mL), Raney-Ni (2 g), 10% palladium on carbon (0.6 g), acetic acid (10 mL), and heating at 50° C. for 4 days with 180 psi of hydrogen. The purification was carried out using reverse-phase -chromatography. The pure fractions were identified by LCMS, collected and lyophilized. 62: MS: M+H⁺=17.

### Synthesis of 1-[(1-phenylethyl]-6-ethoxycarbonyl-4-methyl-3,4-didehydropiperidine (63)

α-Methylbenzylamine (20 g) was dissolved in toluene (60 mL) and 50% ethylglyoxalate in toluene (20 mL). The flask was equipped with magenetic stir bar and Dean-Stark trap. The solution was refluxed (oil bath at 110° C.) for 90 minutes and cooled to room temperature. The crude reaction mixture was evaporated at 35° C. to yield a dark red oil, to this was added methylene chloride (150 mL), followed by addition of isoprene (22.5 g). The mixture was cooled to −65° C. using a cryocool, and to this was added, dropwise, a mixture of trifluoroacetic acid (19 g) and BF₃.Et₂0 (23.5 g). The temperature of the reaction solution was kept in the range of −65° C. to −55° C., and the reaction was stirred at −65° C. for 90 minutes, and was then allowed to warm up to −15° C., followed by the addition of water and sodium bicarbonate to adjust pH of the mixture to 8. The organic layer was separated from the aqueous layer, and subsequently dried over MgSO₄. After evaporation, a red oil was obtained. The oil was filtered over silicagel using 95% hexanes/ethylacetate. After evaporation, a yellow oil was obtained which was crystallized from hexane at −75° C. The solids were filtered, and subsequently recrystallized again from cold hexane to afford 1-[(1-phenylethyl]-6-ethoxycarbonyl-4-methyl-3,4-didehydropiperidine (63) as an off-white crystalline solid (8.3 g). 63: MS: M+H⁺: 274.

### Synthesis of 1-[(1-phenylethyl]-6-ethoxycarbonyl-4-methyl 1-3,4-didehydropiperidine (64)

Ethyl 4,5-dehydro-4-methylpipecolate (63) (2 g, 7.3 mmol) were dissolved in THF (40 ml). The reaction mixture was cooled to −78° C., followed by dropwise addition of 1 M solution of BH₃. THF (21.9 mL, 21.9 mmol). The mixture was allowed to reach 0° C., and was stirred for 1 h at 0° C. 3N aqueous solution of NaOH (7.3 mL, 21.9 mmol) was added dropwise, followed by addition of 30% H₂O₂ (˜2.5 mL, 21.9 mmol). The mixture was stirred at room temperature for 2 h. Water (20 mL) was added, and THF was evaporated under reduced pressure, and the final product was extracted using ethyl acetate. A clear oil was obtained which was purified by flash-chromatography, and the fractions containing the desired final product were identified using LCMS. 64: MS: M+H⁺: 292. ¹H NMR (500 MHz, CDCl₃): δ 7.4-7.2 (m, 5Hₐ), 4.2(t, 3H), 3.96 (m, 1H), 3.4(m, 1H), 3.18(m, 1H), 2.69(m, 1H), 2.0-1.3 (m, 4H), 1.3 (m, 3H), 1.0 (d, 3H).

### Synthesis of 5-hydroxy-4-methyl-2-piperidine carboxylic acid (65)

The compound 64 was subjected to base hydrolysis in ethanol using 2 equivalents of 2N NaOH for overnight. The intermediate obtained from this reaction, N-phenylethyl-protected hydroxy-piperidine carboxylic acid, was hydrogenated (H₂, Pd/C 10%) overnight in ethanol/water. After filtration, the final product was lyophilized, purified by reverse phase chromatography (100% water), and lyophilized to obtain pure 5-hydroxy-4-methyl-2-piperidine carboxylic acid (65). 65: MS: M^{+H}+=160.

### Synthesis of N-(2-hydroxypropyl)-L-valine ethyl ester (67)

To a suspension of L-valine (2 g) in ethanol (50 mL) cooled to −10° C., was slowly added thionyl chloride (2 equivalents). The reaction mixture was then refluxed for 4 hours, and then left to stir overnight. After removal of solvents under reduced pressure, ethanol was added and the resultant suspension was concentrated again. The desired final product (66) (quantitative yield) was further dried in a dessicator over NaOH. 66: MS: M+H⁺=146. Above ethyl ester (2 g) was then dissolved in water (10 mL) in a sealed Pyrex tube, and to this was added propylene oxide (2 g). The reaction mixture was stirred at 50° C. for 4 h, then cooled, concentrated under reduced pressure and lyophilized. The crude product was purified by reverse-phase column chromatography to afford N-(2-hydroxypropyl)-L-valine ethyl ester (67) (1.5 g). 67: MS: M+H⁺=204. The Ddisubstituted compound (68) was also isolated from the reaction mixture.

### Synthesis of N-(2-hydroxypropyl)-L-valine (69)

The hydrolysis of N-(2-hydroxypropyl)-L-valine ethyl ester (67) was carried out in ethanol using 2N aqueous KOH (4 equivalents). The resulting mixture was then heated at 50° C. for 4 days. The mixture was evaporated, and water was added. The reaction product was neutralized to pH 7 using HCl (0.5N). The mixture was lyophilized, and subsequently purified by reverse-phase column chromatography to give N-(2-hydroxypropyl)-L-valine (69) (1.02 g, 34%). 69: MS: M+H⁺=176.

### Synthesis of N-Boc trans-4-hydroxyproline (71)

trans-4-hydroxyproline (70) (5 g, 38 mmol) was dissolved in dioxane/water (1:1) (50 mL), and to the solution was added NaHCO₃ (80 mmol) and Boc anhydride (30 mmol, 6.5 gram). The reaction was stirred for 4 hours. NaHCO₃ was added to keep the pH above 7. The crude reaction mixture was acidified using 0.5 N HCl. Dioxane was evaporated. Boc-trans-4-hydroxyproline was recovered by extraction using EtOAc/water. The organic phase was dried using MgSO₄ and subsequently evaporated to yield N-Boc-4-hydroxyproline (71) as a clear oil (5.6 g, 82%).

### Synthesis of Compound 72

A solution containing N-Boc-trans-4-hydroxyproline (71) (5 g, 21.6 mmol) and triphenylphosphine (11.8 g, 45 mmol) in anhydrous THF (150 mL) was cooled to 4° C. in an ice bath. To this solution was added DEAD (6.5 mL, 45 mmol). The reaction was allowed to stir at room temperature for 24 hours. The reaction mixture was evaporated to give a yellow oil. The crude product was purified by silica gel column chromatography to give of the desired cyclic lactone (72) (2.1g, 45%).

### Synthesis of Compound 73

The cyclic lactone (72) (2.1 g, 9.8 mmol) was dissolved in dry methanol (100 mL). To the solution was added sodium azide (2.34 g, 36 mmol). The reaction mixture was heated overnight at 45° C. After evaporation of the crude reaction mixture, the obtained oil was purified by silica gel column chromatography to give N-Boc-cis-4-hydroxyproline methyl ester (73) (1.3 g, 54%).

### Synthesis of Compound 74

N-Boc-cis-4-hydroxyproline methyl ester (73) (1.3 g, 5.3 mmol) was dissolved in ethanol (20 mL). To the solution was added 2N NaOH aqueous solution (5.3 mL, 10.6 mmol). The reaction was completed after 4 h, and was acidified with 10% citric acid. Ethanol was evaporated, and the final product recovered by extraction with ethylacetate/water. The organic layer was dried over sodium sulfate, filtered and concentrated to yield N-Boc-cis-4-hydroxyproline (74) (960 mg, 78%)

### Synthesis of Compound 75

N-Boc-cis-4-hydroxyproline (74) (500 mg) was dissolved in 30% TFA/methylene chloride (10 mL). The reaction was stirred for 1h and then concentrated under reduced pressure. Water (50 mL) was added, and cis-4-OHproline TFA salt was recovered by lyophilization to yield a yellowish solid. The yellow solid was treated with ether and acetone. The solid was redissolved three times in 50 mL water and lyophilized to obtain cis-4-hydroxyproline (75) (260 mg) as an off-white solid. 75: MS: M+H⁺=132. ¹H NMR (500 MHz, D₂O): δ 4.6 (m, 1H), 4.23 (m, 1H), 3.5 (m, 1H), 3.39 (m, 1H), 2.53 (m, 1H). The ent-75 (compound 201) can be synthesized following the synthetic route (70→75) using D-N-Boc-cis-4-hydroxyproline.

### Synthesis of cis-4-hydroxyproline methyl ester HCl salt (76)

Boc-cis-4-hydroxyproline (74) (450 mg, 1.95 mmol) was dissolved in methanol (10 mL) and cooled to 0° C. To the above solution, 1.8 equivalents of thionyl chloride was added. The solution was heated to 45° C. for 4 hours, and was then stirred overnight at room temperature. The reaction mixture was then concentrated under reduced pressure. cis-4-hydroxyproline methyl ester HCl salt started to crystallize out during the evaporation. The crystals were filtered off and washed several times with ether. The crystals were finally dried in a vacuum oven for 24 hours (40° C.) to yield 76 (354 mg, ˜100%). 76: MS: M+H⁺=146. ¹H NMR (500 MHz, D₂O): δ 4.47 (m, 2H), 3.91 (s, 3H, OMe), 3.52 (m, 2H), 2.57-2.47 (m, 2H). The ent-76 (compound 202) can be synthesized following the synthetic route (70→74, 74→76) using D-N-Boc-cis-4-hydroxyproline.

### Synthesis of N-(-hydroxypropyl)-L-phenylalanine (77)

To a suspension of L-phenylalanine (1 g, 6 mmol) in water (20 mL) in a capped pyrex tube, was added propylene oxide (10 mL), followed by addition of 48% HBr (1 mL). The suspension was heated at 80° C. for 15 min, and then at ambient temperature for 18 h. The reaction mixture was filtered, and the crude product was purified by reverse-phase chromatography to yield the desired N-(2-hydroxypropyl)-L-phenylalanine (77). 77: MS: M+H⁺=224. The disubstituted compound (78) was also isolated from the reaction mixture.

### Synthesis of Compounds 79 and 80

A suspension of (2S,3R,4S)-4-hydroxyisoleucine (496.2 mg, 3.4 mmol) and Cs₂CO₃ (1.1 g, 3.4 mmol) in DMF:H₂0 (10:1) was stirred at room temperature for 15 min before heating to 40-45° C., followed by portion-wise addition of benzyl bromide (1.2 mL, 10.2 mmol). The reaction mixture was stirred at 40-45° C. for 48-110 h, and then cooled to room temperature. After the addition of water (20 mL), the product was extracted with ethyl acetate (5×10 mL) and concentrated under vacuum to obtain crude product. The crude was purified by silica gel column chromatography (ethyl acetate: hexanes, 20:80) to obtain compound 79 (436 mg, 31% yield) as a clear liquid and compound 80 (425 mg, 30% yield) as a clear liquid. 79: ¹H NMR (500 MHz, D₂O): δ 0.66 (d, J=6.40 Hz, 3H), 1.06 (d, J=6.18 Hz, 3H), 2.14 (m, 1H), 3.19 (d, J=13.32 Hz, 2H), 3.37 (m, 2H), 4.10 (d, J=13.16 Hz, 2H), 5.21 (d, J=11.75 Hz,1H), 5.34 (d, J=12.33 Hz, 1H), 7.23-7.32 (m, 10H), 7.34-7.44 (m, 3), 7.47 (d, J=7.65 Hz, 2H). Compound 80: ¹H NMR (500 MHz, CDCl₃): δ 1.23 (d, J=7.30 Hz, 3H), 1.34 (d, J=5.90 Hz, 3H), 2.10 (m, 1H), 3.58 (d, J=10.14 Hz, 1H), 3.78 (s, 4H), 4.25 (m, 1H), 7.25 (m, 2H), 7.33 (t, J=7.45 Hz, 4H), 7.44 (d, J=7.51 Hz, 4H).

### Synthesis of Compound 81

Compound 79 (218 mg, 0.5mmol), N-methyl morpholine N-oxide (91.5 mg 0.7 mmol) and powdered 4 Å molecular sieves (266 mg) were placed in a flame dried flask under nitrogen atmosphere, and to this was added a 2:1 mixture of anhydrous acetonitrile and dichloromethane (3 ml). Tetrapropylammonium perruthennate (19.6 mg, 0.02 mmol) was added to the above suspension and the progress of the reaction was followed by TLC. After concentrating the reaction mixture under reduced pressure, the crude was taken up in dichloromethane and filtered through a pad of silica and the pad was washed with ethyl acetate. After removal of the solvent on rotary evaporator and drying, compound 81 (213 mg, 98% yield) was obtained as a clear oil. Compound 81: ¹H NMR (500 MHz, CDCl₃): δ 0.95 (d, J=6.59 Hz, 3H), 1.73 (s, 3H), 3.15 (m, 1H), 3.25 (d, J=13.39 Hz, 2H), 3.59 (d, J=11.40 Hz, 2H), 3.94 (d, J=13.55 Hz, 2H), 5.23 (d, J=12.19 Hz, 1H), 5.32 (d, J=12.25 Hz, 1H), 7.19-7.29 (m, 10H), 7.36-7.47 (m, 5H).

### Synthesis of Compound 82

A solution of compound 81 (44.4 mg, 0.1 mmol) in a 96:4 mixture of MeOH:HCOOH (1 mL) was added to a suspension of Pd-C (44.4 mg) again in a 96:4 mixture of MeOH:HCOOH (2.5 mL). The reaction mixture was stirred at room temperature for 30 min before adding more of HCOOH (0.5 mL), and the progress of the reaction was monitored by HPLC. The reaction mixture was filtered through filter paper, and solvent was removed on the rotary evaporator to obtain compound 82 (10 mg, 63% yield) as a white solid. Compound 82: ¹H NMR (500 MHz, D₂O): ¹H NMR (500 MHz, D₂O): δ 1.33 (d, J=7.46 Hz, 3H), 2.30 (s, 3H), 3.39 (m, 1H), 4.03 (d, J=3.94 Hz, 1H).

### Synthesis of Compound 83

To a solution of compound 81 (80 mg, 0.19 mmol) in anhydrous THF (1.6 mL) at 0° C. was added slowly a 3M solution of MeMgl in THF (0.29 mL, 0.29 mmol). The reaction mixture was stirred for 4 h and then the reaction was quenched with a saturated aqueous solution of ammonium chloride (3 mL), followed by extraction with ethyl acetate (5×3 mL). The organic phase was concentrated under vacuum to obtain the crude product, and the crude was purified by silica gel column chromatography (ethyl acetate: hexanes, 10:90) to obtain compound 83 (40 mg, 48% yield). Compound 83: ¹H NMR (500 MHz, CDCl₃): δ 1.16 (d, J=7.50 Hz, 3H), 1.23 (s, 3H), 1.32 (s, 3H), 2.32 (quint, J=7.88 Hz, 1H), 3.82 (d, J=14.26 Hz, 2H), 4.01 (d, J=8.89 Hz, 2H), 4.05 (d, J=14.12 Hz, 2H), 7.25 (dd, J=6.32 Hz, J=8.27 Hz, 2H), 7.33 (t, J=7.45 Hz, 4H), 7.44 (d, J=7.51 Hz, 4H).

### Synthesis of Compound 84

A solution of compound 83 (56 mg, 0.17 mmol) in a 96:4 mixture of MeOH:HCOOH (1 mL) was added to a suspension of Pd/C (56 mg) again in a 96:4 mixture of MeOH:HCOOH (2.5 mL). The reaction mixture was stirred at room temperature for 30 min before adding more of HCOOH (0.5 mL), and the progress of the reaction was monitored by HPLC. The reaction mixture was filtered through filter paper, and solvent was removed on the rotary evaporator to obtain compound 84 (8 mg, 73% yield) as a white solid. Compound 84: ¹H NMR (500 MHz, D₂O): δ 1.11 (d, J=7.21 Hz, 3H), 1.51 (s, 3H), 1.57 (s, 3H), 2.89 (quint, J=7.5 Hz, 1H), 4.87 (d, J=7.81 Hz, 1H).

### Synthesis of Compound 85

A solution of 84 (25 mg, 0.17 mmol) in ethanol (0.5 mL) was added to an aqueous solution of LiOH (0.5 M, 0.5 mL, 0.24 mmol) and the reaction mixture was stirred at room temperature for 30 min. pH of the reaction mixture was made ˜7 with careful addition of aqueous HCl (0.1M), and after dilution with more water, the mixture was freeze-dried to obtain compound 85 (25 mg, 90% yield) as a white solid. Compound 85: ¹H NMR (500 MHz, D₂O): δ 1.06 (d, J=7.17 Hz, 3H), 1.29 (s, 3H), 1.42 (s, 3H), 2.03 (quint, J=6.69 Hz, 1H), 3.97 (d, J=5.36 Hz, 1H).

### Synthesis of Compound 87

To a solution of imine 1 (200 mg, 0.97 mmol) in dry DMF (2 mL) under argon at 0° C. was added 1-bromo-3-methylbut-2-ene (86a) (146 μL, 1.26 mmol), followed by addition of Zn (82 mg, 1.26 mmol) and a drop of TMSCl. The reaction mixture was allowed to warm to room temperature over a period of 45 min. After cooling to 0° C., the reaction mixture was neutralized with satd. NH₄Cl, and extracted with diethyl ether (3×50 mL). The organic phase was washed with brine, dried over Na₂SO₄, filtered through a cotton swab, concentrated, and purified by silica gel column chromatography (ethyl acetate/hexanes, 10/90) to obtain compound 87 (2.89 g, 83% yield) as an orange oil. The same procedure produces compound 88 when the starting material is 1-bromo-2-methylbut-2-ene (86b) instead of 1-bromo-3-methylbut-2-ene (86a).

### Synthesis of Compound 89

To a solution of iodosobenzene diacetate (930 mg, 2.8 mmol) in dry MeOH (9.5 mL) under argon was added over a period of 30 min a solution of alkene intermediate 87 (200 mg, 0.61 mmol) in dry MeOH (1.5 mL). After stirring the reaction mixture at room temperature for 30 min, it was neutralized with 1 N HCl (25 mL). The reaction mixture was stirred for another 90 min and extracted with CH₂Cl₂ (2×40 mL), followed by washing of the organic phase with 0.1 M HCl (25 mL). CH₂Cl₂ (20 mL) was added to the combined aqueous acidic phases, and the mixture was basified to pH 8-9 with the addition of solid Na₂CO₃ followed by the addition of di-tert-butyldicarbonate (788 mg, 3.6 mmol). The reaction mixture was stirred for 90 min before decanting the aqueous phase and extracting it with CH₂Cl₂ (2×40 mL). The combined organic phases were dried over Na₂SO₄, filtered through a cotton swab, concentrated, and purified by silica gel column chromatography (ethyl acetatelhexanes, 10/90) to obtain compound 89 (106 mg, 54% yield) as a yellowish orange oil. The same procedure produces compound 90 when the starting material is compound 88 instead of compound 87.

### Synthesis of Compound 91

To a solution of compound 89 (707 mg, 2.6 mmol) in a 1:1 mixture of THF:EtOH (10 mL) was added 1 N NaOH solution (83.2 mL, 83.2 mmol) and the mixture was heated to reflux for 12 h. The reaction mixture was cooled to room temperature, concentrated, and extracted with ethyl acetate (2×50 mL). The organic phase was dried over Na₂SO₄, filtered through a cotton swab, and concentrated to obtain unreacted compound 89. The aqueous phase was acidified to pH 2 with careful addition of 1 N HCl, and extracted with ethyl acetate (3×50 mL). The combined organics were dried over Na₂SO₄, filtered through a cotton swab, and concentrated to obtain compound 91. After repeating the above process on the recovered compound 89, the total yield of compound 91, which is obtained as a white solid, was 445.5 mg (72% yield). The same procedure produces compound 92 when the starting material is compound 90 instead of compound 89.

### Synthesis of Compound 93

To a solution of compound 91 (741 mg, 3 mmol) in dimethoxyethane (30 mL) under argon at −20° C. (ice/MeOH mixture) was added N-iodosuccinimide (1.05 g, 4.6 mmol) in portions. The reaction mixture was stirred at room temperature for 12 h, neutralized with brine, and extracted with diethyl ether (3×50 mL). The combined organics were washed with a satd. aqueous solution of Na₂S₂O₅, dried over Na₂SO₄, filtered through a cotton swab, and concentrated to obtain iodolactone intermediate 93 (1.108 g, 98% yield) as a pinkish solid. The same procedure produces compound 94 when the starting material is compound 92 instead of compound 91.

### Synthesis of Compound 95

To a solution of iodolactone 93 (705 mL, 1.9 mmol) in distilled benzene (5 mL) under argon atmosphere were added tetrabutyltin hydride (824 μL, 3 mmol) and AIBN (recrystallized form MeOH, 43.4 mg, 0.19 mmol). The reaction mixture was heated to reflux for 6 h. CCl₄ (5 mL) was added to the reaction mixture and heating was continued at reflux for another 12 h. The reaction mixture was cooled, concentrated under vacuum, and the crude was purified by silica gel column chromatography (ethyl acetate/hexanes, 10/90) to obtain compound 95 (406 mg, 88% yield) as a white solid. The same procedure produces compound 96 when the starting material is compound 94 instead of compound 93.

### Synthesis of Compound 97

To a stirred solution of compound 95 (210 mg, 0.87 mmol) in dry CH₂Cl₂ at 0° C. under argon was added trifluoroacetic acid (2.34 mL, 30 mmol) and the mixture was allowed to warm to room temperature over a period of 4 h. After concentrating the reaction mixture, amino lactone intermediate 97 (205 mg, 93% yield) was obtained as a white solid. The same procedure produces compound 98 when the starting material is compound 96 instead of compound 95.

### Synthesis of a racemic mixture of (2S,4S)- and (2R,4R)-2-amino-4-hydroxy-3,3-dimethylpentanoic acid (compounds 99a and 99b)

To a solution of amino lactone 97 (144 mg, 0.56 mmol) in distilled water (1.7 mL) was added LiOH (34 mg, 1.4 mmol). The mixture was stirred at room temperature for 25 min and the pH of the reaction mixture was adjusted to 6-7 by the careful addition of acetic acid. The reaction mixture was then concentrated under vacuum. To remove residual water, the crude produce was dissolved in absolute EtOH and concentrated again under vacuum, followed by a repeat of this process for three additional times. The crude product was recrystallized from a minimum amount of EtOH at −20° C. The solid was filtered off and washed with cold EtOH to obtain a racemic mixture of (2S,4S)- and (2R,4R)-2-amino-4-hydroxy-3,3-dimethylpentanioc acid (compounds 99a and 99b) (66 mg, 73% yield) as a white solid. ¹H NMR (200 MHz, D₂O): δ 1.04 (2s, 3H), 1.05 (2s, 3H), 1.22 (d, J=6.34 Hz, 3H), 3.65 (s, 1H), 3.83 (q, J=6.10 Hz, 1H). ¹³C (75 MHz, D₂O): δ 17.30, 20.16, 21.68, 38.47, 62.05, 73.93, 173.60. IR (KBr): 3191, 2973, 2880, 1610, 1492, 1398, 1344, 1105 cm⁻¹. MS (m/z): 162 (M+1), 184 (M+Na), 323 (2M+1).

### Synthesis of racemic mixtures of (2S,3S) and (2R,3R)-2-amino-4-hydroxy-3,4-dimethylpentanoic acid (100a & 100b) and (2S,3R) and (2S,3R)-2-amino-4-hydroxy-3,4-dimethylpentanoic acid (101a & 101b)

The procedure used for the synthesis of compounds 100 (a & b) and 101 (a & b) was identical to those used for compound 99, except that amino lactone 98 was used as the starting material instead of compound 97.

The physical and NMR data of a mixture of compounds 100a & 100b is as follows:

¹H NMR (300 MHz, D₂O): δ 1.01 (d, J=7.17 Hz, 3H), 1.25 (s, 3H), 1.37(s, 3H), 1.98 (m, 1H), 3.93 (d, J=5.61 Hz, 1H). ¹³C NMR (50 MHz, D₂O): δ 11.32, 25.19, 29.16, 43.59, 57.41, 73.86, 174.57. IR (KBr): 32982, 2924, 2659, 1783, 1629, 1527, 1471, 1393, 1278, 1172, 1134, 1061, 934, 549 cm⁻¹. MS (m/z): 162 (M+1), 184 (M+Na), 323 (2M), 345 (2M+Na).

The physical and NMR data of a mixture of compounds 101a & 101b is as follows:

¹H NMR (200 MHz, D₂O): δ 1.01 (d, J=7.34 Hz, 3H), 1.33 (s, 3H), 1.41 (s, 3H), 2.19 (m, 1H), 4.16 (d, J=5.61 Hz, 1H). ¹³C NMR (50 MHz, D₂O): δ 8.17, 25.07, 28.03, 46.14, 56.52, 73.64, 174.91. IR (KBr): 3400, 3120, 3036, 2975, 1781, 1692, 1620, 1598, 1499, 1393, 1356, 1185, 1148, 1083, 942, 883, 680, 531 cm⁻¹. MS (m/z): 162 (M+1), 184 (M+Na), 323 (2M+1), 345 (2M+Na).

### Synthesis of 2-amino-3,4-dimethylpent-4-enoic acid (Compound 102a)

A solution of compound 92 (450 mg, 1.85 mmol) in a 1:3 mixture of 1 N HCl:HCOOH (2.9 mL) was stirred at 50° C. for 12 h. After cooling the reaction mixture to room temperature, toluene (1 mL) was added and the mixture was concentrated under vacuum to remove HCOOH, and this process was repeated twice more. The crude mixture was freeze-dried for 12 h, diluted with a minimum amount of ethyl acetate (250 μL), and treated with excess propylene oxide (3.5 mL). The reaction mixture was stirred for 6 h at room temperature and filtered. The precipitates were washed with hexanes, and freeze-dried for 12 h to obtain a racemic mixture of diastereoisomers of 2-amino-3,4-dimethylpent-4-enoic acid (compound 102a) (186 mg, 70% yield) as a white solid. ¹H NMR (300 MHz, D₂O): 1.06 (d, J=7.17 Hz, 3H), 1.13 (d, J=7.17 Hz, 3H), 1.71 (s, 3H), 1.81 (s, 3H), 2.64 (m, 1H), 2.83 (m, 1H), 3.55 (d, J=8.64 Hz, 2H), 3.88 (d, J=3.75 Hz, 1H), 4.92 (s, 1H), 4.94 (s, 1H), 5.01 (s, 1H), 5.06 (s, 1H). ¹³C NMR (50 MHz, D₂O): δ 12.17, 16.09, 18.79, 21.04, 40.67, 42.90, 56.52, 57.91, 113.84, 114.94, 144.81, 145.01, 174.26, 174.45. IR (KBr): 3092, 2976, 2672, 2102, 1626, 1589, 1516, 1401, 1327, 1185, 901, 716 cm⁻¹. MS (m/z): 166 (M+Na), 287 (2M). Anal. Calcd for C₇H₁₃NO₂: C, 58.72; H, 9.15; N, 9.78. Found: C, 58.53; H, 9.02; N, 9.61.

Similarly, 102b was synthesized from compound 91. Compound 102b: ¹H (300 MHz, D₂O): δ 1.06 and 1.13 (2d, J=7.17 Hz, 3H, H₆, H₆), 1.71 and 1.81 (2s, 3H, H₇ et H₇), 2.64 and 2.83 (2m, 1H, H₃ et H₃ ), 3.55 (d, J=8.64 Hz, 2H, NH₂), 3.88 (d, J=3.75 Hz, 1H, H₂), 4.92, 4.94, 5.01, 5.06 (2×2s, 1H, H₅ et H₅). ¹³C NMR (50 MHz, D₂O): δ 12.17, 16.09, 18.79 21.04, 40.67, 42. 90, 56.52, 57.91, 113.84, 114.94, 144.81, 145.01, 174.26, 174.45. IR (KBr): 3092, 2976, 2672, 2102, 1626, 1589, 1516, 1401, 1327, 1185, 901, 716 cm⁻¹. MS (m/z): 166 (M+Na), 287 (M+M).

### Synthesis of Compound 103

(2S,3R,4S)-4-hydroxyisoleucine (100 mg, 0.68 mmol) was heated to reflux in aqueous HCl (6N) or HBr for 6 h. The reaction mixture was cooled to room temperature and neutralized using aqueous NaOH to pH 7. After concentration, the crude was purified using silica gel chromatography (ethyl acetate:hexanes, 1:4) to give compound 103 (62 mg, 70% yield) as a white solid. ¹H NMR (500 MHz, CDCl₃): δ 1.24 (d, J=7.42 Hz, 3H), 1.52 (d, J=7.10 Hz, 3H), 2.85 (quint, J=7.42 Hz, 1H), 4.71 (m, 2H).

### Synthesis of Compound 104

Compound 103 (100 mg, 0.48 mmol) was dissolved in pyridine (2 mL), followed by addition of acetic anhydride (0.07 ml, 0.718 mmol), and the above mixture was stirred at room temperature for overnight. After concentrating, the residue was taken up in water and pH was adjusted to 34 with aqueous HCl (0.1M). The aqueous phase was extracted with ethyl acetate (4×5 ml) and concentrated. Recrystallization from hexanes/ethyl acetate gave compound 104 (18 mg, 22% yield) as a white solid. Compound 104: ¹H NMR (500 MHz, CDCl₃): δ 4.74 (1H, dd, J=5.57 Hz, J=7.65 Hz), 4.41 (1H, quad, J=6.64 Hz), 2.68 (1H, quint, J=7.42 Hz), 2.08 (3H, s), 1.45 (3H, s), 0.95 (3H, d, J=7.30 Hz).

### Synthesis of Compound 105

Pyridine (0.12 mL, 1.44 mmol) was added to a solution of compound 103 (100 mg, 0.48 mmol) in anhydrous CH₂Cl₂ (2 ml), and the mixture was cooled to 0° C. followed by the addition of benzoyl chloride (0.06 ml, 0.53 mmol). The reaction mixture was stirred at 0° C. for 1 h, overnight at room temperature, and then under refluxed for 5.5 h. More pyridine (0.48 mmol) and benzoyl chloride (0.48 mmol) were added to the cooled mixture, which was left stirring overnight. The reaction mixture was diluted with ethyl acetate (5 mL), washed with 1 N HCl (4×8 mL) until the pH was 3-4. The organic phase was washed with saturated NaHCO₃ (5 mL) to pH 8, followed by water (5 mL). The organic layer was concentrated and the crude was recrystallized from hexanes/ethyl acetate to give compound 105 (40 mg, 36% yield) as a white solid. Compound 105: ¹H NMR (500 MHz, CDCl₃): δ 7.82 (2H, d, J=8.0 Hz), 7.55 (1H, t, J=7.41 Hz), 7.47 (2H, t, J=7.62 Hz), 4.92 (1H, dd, J=5.29 Hz, J=8.02 Hz), 4.47 (1H, quad, J=6.6 Hz), 2.84 (1H, quint, J=7.34 Hz), 1.51 (3H, d, J=7.05 Hz), 1.02 (3H, d, J=7.36 Hz).

### Synthesis of Compound 106

To a solution of compound 103 (100 mg, 0.48 mmol) and triethylamine (0.067 mL, 0.48 mmole) in anhydrous THF (1.8 mL) at 0° C. was added benzaldehyde (0.07 mL, 0.71 mmol) and sodium triacetoxyborohydride (149 mg, 0.67 mmol) in succession. The reaction mixture was stirred at 0° C. for 3 h and extracted with ethyl acetate (4×5 ml) after the addition of water (10ml). The organic phases were combined and concentrated under vacuum to obtain crude product. The crude product was purified by silica gel column chromatography (ethyl acetate: hexanes, 1:4) to obtain compound 106 (45 mg, 43% yield) as a white solid. Compound 106: ¹H NMR (500 MHz, CDCl₃): δ 7.3-7.2 (5H, m), 4.0 (3H, m), 3.2 (1H, d, J=Hz), 2.0 (1H, m), 1.4 (3H, d, J=Hz), 1.1 (3H, d, J=Hz).

### Synthesis of Compounds 107a,b and 108a,b

To a solution of compound 103 (1 g, 4.76 mmol) in dichloromethane (15 mL) at 0° C. was added triethylamine (2 mL, 14.3 mmol) and after 15 min, p-toluenesulfonyl chloride (1.36 g, 7.14 mmol). The resulting mixture was slowly warmed to room temperature and then stirred overnight. The reaction mixture was extracted with dichloromethane (5×10 mL) and ethyl acetate (2×10 mL) after addition of water (30 mL). The organic phase was combined, washed with saturated aqueous NaHCO₃ and brine, and concentrated under vacuum to obtain crude product as an orange residue. The crude was purified by silica gel column chromatography (ethyl acetate: hexanes, range varying from 5:95 to 25:75) to obtain 107a (982 mg, 73% yield) as a white solid and 108a (31 mg, 15% yield) as a white solid. 107a: ¹H NMR (500 MHz, CDCl₃): δ 7.79 (2H, d, J=8.17 Hz), 7.34 (2H, d, J=8.20 Hz), 4.83 (1H, d, J=3.59 Hz), 4.37 (1H, q, J=6.72 Hz), 4.10 (1H, dd, J=3.95 Hz, J=7.53 Hz), 2.54 (1H, quint, J=7.27 Hz), 2.44 (3H, s), 1.37 (3H, d, J=6.95 Hz), 1.08 (3H, d, J=8.08 Hz). 108a: ¹HNMR (500 MHz, CDCl₃): δ 7.98 (2H, d, J=8.14 Hz), 7.32 (4H, dd, J=8.08 Hz), 7.16 (2H, d, J=7.95 Hz), 4.78 (1H, d, J=11.29 Hz), 4.52 (1H, m), 2.47 (3H, s), 2.40 (3H, s), 2.34-2.17 (1H, m), 1.41 (3H, d, J=6.26 Hz), 1,15 (3H, d, J=7.28 Hz). The synthesis of the N-Cbz derivatives 107b and 108b follows the above synthetic route using either Cbz-Cl or Cbz-anhydride as electrophile.

### Synthesis of Compound 109

To a solution of compound 103 (1 g, 4.76 mmol) in dichloromethane (15 mL) at 0° C. was added triethylamine (2 mL, 14.3 mmol) and o-nitrobenzenesulfonyl chloride (1.62 g, 7.14 mmol). The resultant mixture was allowed to warm to room temperature and stirred overnight. Water (30 mL) was added and the mixture was stirred for 1 h. The crude was extracted with dichloromethane (5×15 mL) and ethyl acetate (15 mL). The organic phase was combined, washed with saturated aqueous NaHCO₃ (30 mL) and brine, (70 mL) and concentrated. The crude was purified by silica gel column chromatography to obtain compound 109 (0.77 g, 65% yield) as a white solid. Compound 109: ¹H NMR (500 MHz, CDCl₃): δ 1.17 (d, J=7.43 Hz, 3H), 1.42 (d, J=6.39 Hz, 3H), 2.57 (quint, J=7.44 Hz, 1H), 4.40 (m, 2H), 5.94 (d, NH, 1H), 7.77 (dd, J=3.36 Hz, J=5.54 Hz, 2H), 7.97 (t, J=4.51 Hz, 1H), 8.15 (dd, J=3.57 Hz, J=5.31 Hz, 1H).

### Synthesis of Compound 110

To a solution of compound 109 (476 mg, 1.51 mmol) in anhydrous dichloromethane (8 mL) at 0° C. was dropwise added pyrrolidine (0.38 mL, 4.54 mmol). The mixture was stirred overnight at 5° C., and then for 2 h at room temperature. To the mixture were added dichloromethane (5 mL) and water (4 mL), and the pH was adjusted to 6-7 by careful addition of HCl (1 N), followed by extraction with CH₂Cl₂ (4×5 mL) and ethyl acetate (5 mL). The organic phases were combined, dried over Na₂SO₄ and concentrated to give compound 110 (290 mg, 60% yield) as a white solid. Compound 110: ¹H NMR (500 MHz, CDCl3): δ 0.97 (d, =6.83 Hz, 3H), 1.18 (d, =5.95 Hz, 3H), 1.69 (bs, 1H), 1.77-1.94 (m, 4H), 2.92 (m, 1H), 3.21 (m, 1H), 3.49 (m, 1H), 3.84 (m, 1H), 4.29 (d,=4.58 Hz, 1H), 7.68 (m, 2H), 7.91 (m, 1H), 8.00 (m, 1H).

### Synthesis of Compound 111a,b

To a solution of compound 107a (200 mg, 0.71 mmol) in ethanol (2.6 mL) and THF (0.7 mL) was added dropwise to an aqueous solution of LiOH (33 mg, 0.78 mmol). The reaction mixture was left stirring at room temperatuer for overnight. The pH was adjusted to ˜6 with careful addition of aqueous HCl (1 N) before removal of the solvents. The product was dried under reduced pressure to give compound 111a (207 mg, 98% yield) as a white solid. Compound 111a: ¹H NMR (500 MHz, CDCl₃): δ 7.77 (2H, d, J=7.88 Hz), 7.47 (2H, d, J=7.79 Hz), 3.96 (1H, quint, J=5.75 Hz), 3.49 (1H, d, J=7.77 Hz), 2.46 (3H, s), 1.87 (1H, m), 1.03 (3H, d, J=6.21 Hz), 0.84 (3H, d, J=6.77 Hz). The synthesis of N-CBz derivative (111b) follows the above synthetic route.

### Synthesis of Compound 112a,b

Pyrrolidine (0.18 mL. 2.12 mmol) was dropwise added to a 0° C. cooled solution of compound 107a (200 mg, 0.71 mmol) in anhydrous CH₂Cl₂, and the mixture was stirred for 48 h at 5° C. To the mixture were added CH₂Cl₂ (5 mL) and water (3 mL) and pH was adjusted to ˜6 with careful addition of aqueous HCl (1 N). The crude product was extracted with CH₂Cl₂ (5 mL) and EtOAc (3×5 mL), the organic phases were combined, dried over Na₂SO₄, and concentrated. The crude was purified by silica gel column chromatography to obtain compound 112a (154 mg, 62% yield) as a white solid. Compound 112a: ¹H NMR (500 MHz, CDCl₃): 0.93 (d, J=6.64 Hz, 3H), 1.17 (d, J=5.94 Hz, 3H), 1.58 (m, 1H), 1.70-1.76 (m, 2H), 1.88 (m, 2H), 2.42 (s, 3H), 2.97 (m, 1H), 3.05 (m, 1H), 3.11 (m, 1H), 3.21 (m, 1H), 3.34 (m, 1H), 3.89 (m, 2H), 6.07 (d, J=9.12 Hz, 1H), 7.29 (d, J=7.31 Hz, 2H), 7.73 (d, J=7.59 Hz, 2H). ¹³C-NMR (500 MHz, CDCl₃): δ 14.3, 21.0, 22.4, 24.7, 26.7, 44.5, 46.8, 47.3, 58.2, 68.8, 128.3, 130.3, 137.8, 144.4, 170.9. The synthesis of N-CBz derivative (112b) follows the above synthetic route.

### Synthesis of Compound 113a,b

To a solution of compound 112a (100 mg, 0.28 mmol) in anhydrous CH₂Cl₂ (15 mL) was added PCC (225 mg, 1.17 mmol), and the resultant mixture was stirred overnight at room temperature. The reaction mixture was filtered through a pad of celite, and concentrated. The crude was purified by silica gel column chromatography to obtain compound 113a (86 mg, 82% yield) as an oil. Compound 113a: ¹H NMR (500 MHz, CDCl₃): δ 1.02 (d, J=6.6 Hz, 3H), 1.6 (m, 1H), 1.73 (m, 1H), 1.83 (m, 1H), 2.19 (s, 3H), 2.41 (s, 3H), 2.86 (m, 1H), 3.02 (m, 1H), 3.21 (m, 1H), 3.32 (m, 1H), 4.16 (t, J=8.79 Hz, 1H), 5.62 (bs, 1H), 7.27 (d, J=11.45 Hz, 2H), 7.69 (d, J=8.07 Hz, 2H). The synthesis of N-CBz derivative (113b) follows the above synthetic route.

### Synthesis of Compound 114

To a mixture of (2S,3R,4S)-4-hydroxyisoleucine (442.7 mg, 3.0 mmol) and NaOH (132 mg, 3.3 mmol) in water (11 mL) and t-butanol (6 mL), CbzCl (561 mg, 3.3 mmol) was added dropwise. The resulting reaction mixture was stirred overnight at room temperature. The reaction mixture was acidified to pH 2 by using 1 M HCl. The mixture was extracted with DCM (2×100 mL). The organic phase was dried over Na₂SO₄ and evaporated to provide 114 (790 mg, 99%) as a white solid. 114: ¹H NMR (500 MHz, CDCl₃): δ 1.00 (d, J=7.07 Hz, 3H), 1.44 (d, J=6.31 Hz, 3H), 2.59 (m, 1H), 4.39 (m, 1H), 4.66 (m, 1H), 5.14 (s, 2H), 5.52 (br, 1H), 7.37 (m, 5H).

### Synthesis of Compound 115

Pyrrolidine (0.94 mL, 11.4 mmol) was dropwise added to a solution of compound 114 (1 g, 3.8 mmol) in anhydrous CH₂Cl₂ (10 mL) and the mixture was stirred for 6 h at room temperature. Water (3 mL) was added to the reaction mixture and it was extracted with dichloromethane (4×10 mL) and EtOAc (10 mL). The combined organic phases were washed with aqueous HCl (1 N, 6 mL), dried over sodium sulfate, filtered and concentrated. The crude was purified by silica gel column chromatography (ethyl acetate: hexanes:methanol, 1:1:1/8) to obtain compound 115 (694 mg, 55% yield) as a clear liquid. Compound 115: ¹H NMR (500 MHz, CDCl₃): δ 0.97 (d, J=7.0 Hz, 3H), 1.19 (d, J=6.14 Hz, 3H), 1.81-1.91 (m, 2H), 1.92-2.00 (m, 3H), 3.40-3.58 (m, 4H), 3.60-3.73 (m, 2H), 4.51 (dd, 1H) 5.10 (s, 2H), 5.82 (d, 1H), 7.27-7.32 (m, 5H).

### Synthesis of Compound 116

Pyrrolidine (2.36 mL, 26.8 mmol) was dropwise added over a period of 5 min to a solution of compound 103 (1 g, 4.76 mmol) in anhydrous CH₂Cl₂ (10 mL) and the resultant yellowish mixture was stirred for overnight at room temperature. Water (10 mL) was added to the reaction mixture and pH was adjusted to ˜5 with aquoeous HCl (1 N, 16 mL). The aqueous phase was extracted with dichloromethane (5×10 ml) and EtOAc (10 mL). The combined organic phases were dried over sodium sulfate, filtered and concentrated. The crude was purified by silica gel column chromatography (ethyl acetate: hexanes:methanol, 1:1:1/8) to obtain compound 116 (323 mg, 34% yield) as a white solid. Compound 116: ¹H NMR (500 MHz, CDCl₃): δ 4.60 (1H, d, J=10.43 Hz), 4.28 (1H, d, J=10.31 Hz), 3.69 (1H, m), 3.49 (3H, m), 3.34 (2H, m), 2.26 (1H, bs), 2.00-1.83 (4H, m), 1.74 (1H, m), 1.25 (3H, d, J=7.28 Hz), 0.78 (3H, d, J=6.64 Hz).

### Synthesis of Compound 117

To a solution of compound 116 (100 mg, 0.5 mmol) in anhydrous CH₂Cl₂ (3 mL) at 0° C. was added triethylamine (0.21 mL, 1.5 mmol) and the mixture was stirred for 15 min. p-Toluenesulfonyl chloride (105 mg, 0.55 mmol) was added and the reaction mixture was allowed to warm to room temperature and stirred overnight. Water (6 mL) was added and the mixture was stirred for another 30 min. The aqueous phase was extracted with dichloromethane (3×15 ml) and EtOAc (2×5 mL). The combined organic phases were washed with saturated NaHCO₃ (15 mL) and brine (30 mL), dried over sodium sulfate, filterted and concentrated. The crude was purified by silica gel column chromatography to obtain compound 117 (129 mg, 71% yield) as a white solid. Compound 117: ¹H NMR (500 MHz, CDCl₃): δ 0.75 (d, J=6.62 Hz, 3H), 1.35 (d, J=6.07 Hz, 3H), 1.80-2.07 (m, 4H), 2.42 (s, 3H), 3.09-3.15 (m, 1H), 3.45-3.55 (m, 3H), 3.75 (m, 1H), 3.84 (m, 1H), 4.70 (d, J=10.86 Hz, 1H), 5.44 (d, J=10.62 Hz, 1H), 7.29 (d, J=7.89 Hz, 2H), 7.84 (d, J=7.84 Hz, 2H).

### Synthesis of Compound 118

To a solution of compound 116 (200 mg, 0.94 mmol) in anhydrous THF (4 mL) was added NaH (47 mg, 1.18 mmol), and the mixture was stirred at room temperature for 30 min. Benzyl bromide (177 mg, 1.04 mmol) was added and the reaction mixture was stirred for 15 h. Water (4 mL) was added and the mixture was stirred for another 30 min. The aqueous phase was extracted with dichloromethane (4×4 ml) and EtOAc (4 mL). The combined organic phases were dried over sodium sulfate, filterted and concentrated. The crude was purified by silica gel column chromatography to obtain compound 118 (185 mg) as a white solid. Compound 118: ¹H NMR (500 MHz, CDCl₃): δ 0.81 (d, J=6.31 Hz, 3H), 1.30 (d, J=5.98 Hz, 3H), 1.70-1.82 (m, 1H), 1.86-1.94 (m, 1H), 2.14-2.22 (m, 1H), 3.16-3.21 (m, 1H), 3.26-3.32 (m, 1H), 3.36 (d, J=10.63 Hz, 1H), 3.41-3.46 (m, 2H), 3.73 (d, J=14.24 Hz, 1H), 3.96-3.99 (m, 2H), 4.24 (d, J=10.29 Hz, 1H), 4.44 (d, J=10.24 Hz, 1H), 7.18-7.28 (m, 5H).

### Synthesis of Compound 119

To a solution of compound 103 (1.05 g, 5 mmol) in methanol (20 ml) under nitrogen atmoshphere was added pyrrolidine (2.2 mL, 25 mmol), and the reaction mixture was stirred overnight at room temperature. After removal of the solvent, the crude was purified by silica gel column chromatography (dichloromethane:methanol, 90:10) to provide compound 119 (618 mg, 61% yield) as a white solid. Compound 119: ¹H NMR (500 MHz, CDCl₃): δ 0.90(d, J=6.98 Hz, 3H), 1.87 (d, J=6.11 Hz, 3H), 1.92 (m, 1H), 1.97 (m, 2H), 2.05 (m, 2H), 3.46 (m, 2H), 3.57 (m, 1H), 3.94 (m, 2H), 4.29 (m, 1H). ¹³C NMR(500 MHz, CDCl₃): δ 14.4, 23.3, 25.0, 26.8, 42.7, 47.4, 48.6, 57.9, 73.2, 169.1.

To a solution of compound 119 (50 mg, 0.25 mmol) and triethylamine (0.1 mL, 0.8 mmol) in dichloromethane (3 ml) under nitrogen atmosphere was added a solution of p-toluenesulfonyl chloride (53 mg, 0.28 mmol) in dichloromethane (0.5 mL), and the resultant reaction mixture was stirred overnight at room temperature. After removal of the solvent the crude was purified by silica gel chromatography (dichloromethane:methanol, 80:20) to obtain compound 112 (49 mg, 55% yield) as a pale yellow solid.

### Synthesis of Compound 120

To a solution of compound 119 (50 mg, 0.25 mmol) in dichloromethane (1 mL) at 0° C. under nitrogen atmosphere was added IM solution of LiHMDS in hexanes (0.55 mL, 0.55 mmol). After 15 min at 0° C. the reaction mixture was cooled down to −78° C. and benzyl bromide (213 mg, 1.25 mmol) was added. The reaction mixture was allowed to warm to room temperature and stirred overnight. After completion, the reaction was quenched with methanol, concentrated and the crude was purified by silica gel chromatography to give compound 120 (40 mg, 55% yield) as a colourless liquid. Compound 120: ¹H NMR(500 MHz, CDCl₃): δ 0.77 (d, J=6.98 Hz, 3H), 1.19 (d, J=5.86 Hz, 3H), 1.67 (m, 1H), 1.92 (m, 4H), 3.27-3.37 (m, 3H), 3.51-3.61 (m, 3H), 3.70 (m, 1H), 3.80 (d, J=13.01 Hz, 1H), 7.32 (m, 5H).

### Synthesis of Compounds 121a and 121b

In a round bottom flask, (2S,3R,4S)-4-hydroxyisoleucine (295 mg, 2.0 mmol), Cs₂CO₃ (1.3 g, 4 mmol), BnEt₃NBr (227 mg, 1.0 mmol) and BrCH₂COOEt (0.24 mL, 2.2 mmol) were added in sequence into tBuOMe/H₂O (1:1, 20 mL). The resulting mixture was stirred at 40° C. for 48 h. Then, the pH of the mixture was adjusted to 4. The solvent was removed under reduced pressure, and the crude product eas purified by HPLC to provide compound 121a (360 mg) as a white solid and 121b (20 mg) in overall 92% after freeze-drying. 121s: ¹H NMR (500 MHz, D₂O): δ 3.88 (m, 1H), 3.81 (d, J=5.77 Hz, 1H), 3.53-3.70 (dd, 2H), 1.96 (m, 1H), 1.29 (d, J=6.32 Hz, 3H), 0.98 (d, J=7.22 Hz, 3H). 121b: ¹H NMR (500 MHz, D₂O): δ 3.76-4.08 (m, 6H), 2.10 (m, 1H), 1.37 (d, J=6.50 Hz, 3H), 1.08 (d, J=7.45 Hz, 3H).

### Synthesis of Compound 123

A solution of dibenzyl lactone (122) (154 mg, 0.5 mmol), obtained from (2S,3R,4S)-4-hydroxyisoleucine, in EtOH (3 mL) was added dropwise into LiOH (0.6 mmol, 0.2 M) solution. The resulting mixture was stirred at room temperature overnight and monitored by TLC. After adjustment of the pH to 6, the solvent was removed under reduced pressure, and the crude product was purified by HPLC to provide pure hydrophobic compound 123 (24.5 mg, 15%). A diastereomeric product accounting for 70% of the product was also recovered during purification. 123: ¹H NMR (500 MHz, CD₃OD): δ 7.23-7.40 (m, 10H), 3.82-3.96 (m, 5H), 3.37 (d, J=11.77 Hz, 1H), 2.10 (m, 1H), 1.33 (d, J=6.26 Hz, 3H), 1.00 (d, J=6.26 Hz, 3H), 1.00 (d, J=6.73 Hz, 3H).

### Synthesis of Compound 125

To commercially available (S)-lactate methyl ester (124) (590 mg, 5.0 mmol) and p-toluenesulfonic acid (a few crystals) in THF (5 mL) under nitrogen was added DHP (0.42 mL, 5.5 mmol) dropwise at 0° C. The resulting mixture was stirred at room temperature for 3 h. After evaporation of the solvent, the crude product was purified by silica gel column chromatography to afford 125 (0.86 g, 92% yield) as a clear oil.

### Synthesis of Compound 126

To a solution of compound 125 (752.4 mg, 4.0 mmol) in toluene (25 mL) under nitrogen at −78° C., DIBAL (10 mL, 10.0 mmol, 1.0 M in toluene) was added dropwise. The resulting mixture was stirred at −78° C. for 2.5 h, followed by quenching with the addition of CH₃OH (3 mL). After 5 min, concentrated potassium sodium tartrate solution (25 mL) was added and the resulting mixture was warmed up to room temperature for 15 min. The mixture was extracted with ethyl acetate (300 mL). After removal of solvent under reduced pressure, 126 (620 mg, 98% yield) as a pleasant smelling oil was obtained.

### Synthesis of Compound 127

Above obtained oil (126) was dissolved in methanol (25 mL) at 0° C. with (iPr)₂NEt (0.70 mL, 4.0 mmol) and valine methyl ester hydrochloride (670 mg, 4.0 mmol) and sodium cyanoborohydride (4.0 mL, 4.0 mmol, 1.0 M in THF). The reaction mixture was stirred at room temperature overnight. After evaporation, the crude product was purified by silica gel column chromatography to afford 127 as a clear oil (920 mg, 66%). Other diastereoisomer was also present in the reaction mixture, but was removed by chromatography. 127: ¹H NMR (500 MHz, CDCl₃): δ 0.89 (d, J=6.71 Hz, 3H), 0.91 (d, J=6.80 Hz, 3H), 1.14 (d, J=6.33 Hz, 3H), 1.83-1.89 (m, 5H), 2.33 (m, 1H), 2.58 (m, 1H), 2.94 (m, J=6.35 Hz, 1H), 3.68 (s, 3H), 3.74 (m, 1H), 3.82 (m, 1H), 3.88 (m, 1H), 5.24 (s, 1H).

### Synthesis of Compound 128

To the solution of compound 127 (546.2 mg, 2.0 mmol) in ethanol (2 mL), NaOH (2.5 mL, 2.5 mmol, 1.0 M in H₂O) was added. The resulting mixture was stirred at room temperature overnight. Then, HCl (4 mL, 1.0 M) was added. The resulting mixture was stirred at room temperature for another 4 h. The mixture was evaporated under vacuum. The crude product was recrystallized from 2% methanol in dichloromethane to provide 128 (285 mg, 95% yield) as a white solid. This gave 58% of overall yield for above synthesis. 128: ¹H NMR (500 MHz, CDCl₃): δ 1.06 (d, J=6.92 Hz, 3H), 1.12 (d, J=6.90 Hz, 3h), 1.26 (d, J=6.12 Hz, 3H), 2.37 (m, 1H), 3.02 (m, 1H), 3.24 (d, J=12.92 Hz, 1H), 3.85 (d, 1H), 4.15 (m, 1H).

### Synthesis of Compound 133

The compound 133 (SR) isomer was synthesized following the above mentioned route for SS-isomer starting from (R)-lactate methyl ester (129) in an over all yield of 60%. 133: ¹H NMR (500 MHz, CDCl₃): δ 1.06 (d, J=6.86 Hz, 6H), 1.12 (d, J=7.08 Hz, 3H), 2.33 (m, 1H), 3.03 (m, 1H), 3.21 (d, J=12.96 Hz, 1H), 3.68 (d, J=3.77 Hz, 4.19 (m, 1H).

### Synthesis of Compound 134

Imine 1 (1 eq.) was added dropwise to a mixture of 2-pentanone (22 eq) and L-proline (0.35 eq) in dry DMSO (40 mL) at room temperature under nitrogen, and the mixture was stirred at room temperature for 2 h. The reaction mixture was diluted with phosphate buffer (pH 7.4, 150 mL), followed by extraction with ethyl acetate (3×200 mL). The organic phase was dried over MgSO₄ and concentrated under vacuum. Purification by silica gel column chromatography yielded compound 134 in 72% isolated yield.

### Synthesis of Compound 135

To a solution of compound 134 (10 mmol) in CH₃CN (6 mL) at 0° C., was added a solution of ceric ammonium nitrate (CAN, 3 eq) in water (60 mL) with stirring. The reaction mixture was stirred for 30 min at 0° C. CH₂Cl₂ (60 mL) was added to the reaction mixture and the aqueous phase was separated, and extracted twice with CH₂Cl₂ ; once after making acidis with 0.1 N HCl and once when made neutal (pH 7) with Na₂CO₃ (2N). The combined organic phases were dried over MgSO₄ and concentrated under vacuum to obtain deprotected amine 135 in an isolated yield of 84%.

### Synthesis of Compound 136

To a solution of compound 135 (10 mmol) in MeOH at 0C was added NaBH₄ (12 mmol) and the mixture was stirred for 90 min at 0° C. After the addition of water (40 mL), the reaction mixture was extracted with CH₂Cl₂ (3×90 mL). The combined organics were dried over MgSO₄, filtered, and concentrated under vacuum to yield intermediate 136 in an isolated yield of 89%.

### Synthesis of (2S, 3S, 4S)-2-Amino-4-hydroxy-3-methyl-hexanoic acid (compound 12b)

To a solution of compound 136 (10 mmol) in MeOH/H₂O ( 1/10, 30 mL) was added LiOH (12 mmol). The mixture was stirred at room temperature over night. Acetic acid (12 mmol) was added and the reaction mixture was concentrated. Water was removed from the crude product by repeated addition and evaporation of absolute EtOH. The recrystallization of the crude product from EtOH gave (2S, 3S, 4S)-2-Amino-4-hydroxy-3-methyl-hexanoic acid (compound 12b) in an isolated yield of 50%. ¹H NMR (300 MHz, D₂O): δ 0.97 (m, 6H), 1.55 (m, 1H), 2.23 (m, 2H), 3.56 (m, 1H), 3.99 (d, J=2.8 Hz, 1H). ¹³C NMR (75 MHz, D₂O): δ 9.52, 11.78, 27.48, 38.02, 56.11, 75.38, 174.77. MS (IC) m/z: 162 [M+H]⁺. The compound 13b was also isolated from silica gel column chromatography purification of the filterate and ¹H NMR was in accord with the structure.

C) Additional Analogs of 4-hydroxyisoleucine

Analogs of 4-hydroxyisoleucine in which the 3- and 4-positions are substituted with groups other than methyl can also be prepared using standard chemistry known in the art for synthesizing α-amino acids using commercially available or known precursors. Examples of the synthetic methods that would be employed in such preparations can be found in Rolland-Fulcrand et al., Eur. J. Org. Chem., 873-773, 2004; Kassem et al., Tetrahedron: Assymetry 12:2657-61, 2001; Wang et al., Eur. J. Org. Chem., 834-39, 2002; Tamura et al., J. Org. Chem. 69:1475-80, 2004; Jamieson et al., Org. Biomol. Chem. 2:808-9, 2004; Gull and Scholikopf, Synthesis 1985:1052, 1985; Inghardt et al., Tetrahedron 32:6469-82, 1991; and Dong et al., J. Org. Chem. 64:2657-66, 1999.

### Example 2:

### Stimulation of Glucose Uptake by Differentiated 3T3-L1 Adipocyte cells by Analogs of 4-hydroxyisoleucine

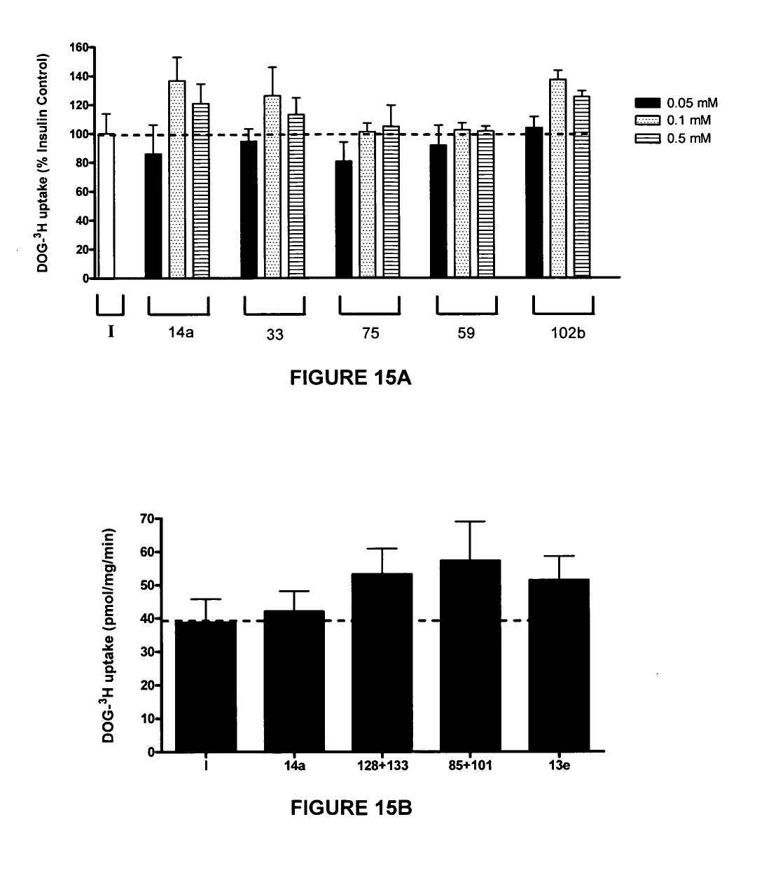

Selected analogs according to the invention were tested for their effect on the uptake of ³H-deoxy-glucose by differentiated 3T3-L1 adipocyte cells. Briefly, 3T3-L1 adipocyte cells (ATCC; Cl-173) were cultured in 12 well tissue culture plates for 3 days in order to reach confluence (Lakshmanan et al., Diabetes Mellitus: Methods and Protocols, Saire Ozcna, Ed., Humana Press Inc., Tonowa, N.J. 97-103, 2003). The culture medium was removed and replaced with differentiation medium (Green and Meuth, Cell 3:127-133, 1974; Madsen et al., Biochem. J. 375:539-549, 2003) and then the cells were incubated for an additional 9 days. The state of differentiation was confirmed by visual examination. Cell starvation was conducted for 5 hours by replacing the differentiation medium with one lacking fetal calf serum. During the last 30 minutes of the starvation period, the cells were exposed to compounds to be assayed at a range of concentrations. As a positive control, cells were exposed to insulin (0.0167 U/mL; Sigma; Cat. No. 15534) for the last 30 minutes of the starvation period Cells were exposed to 0.5 mM isoleucine are used as a control for background uptake. All treatments were performed in triplicate. Cells were washed, then fresh medium containing 16 μM ³H-Deoxy-D-glucose (0.5 μCi/mL) and 10 μM 2-Deoxy-D-glucose was added and the cells were incubated for 10 min. Glucose uptake was stopped by washing the cells with ice cold PBS. The cells were lysed and specific activity in the lysate was determined relative to background uptake of ³H-deoxy-glucose. Results were standardized on the basis of protein content per well. As shown in FIG. 15A, insulin (I) at 10⁻⁷ M strongly promoted glucose uptake as expected. Compound #14a (4-hydroxyisoleucine) stimulated glucose uptake at all three concentrations tested. All the analogs tested stimulated at least minimally the uptake of glucose, Compounds #33 and #102b being the most effective by demonstrating at least equivalent activity to the parent compound.

FIG. 1 5B is another figure showing insulin stimulation of glucose uptake by insulin at 10⁻⁷M, and by the analogs of the invention. At 0.5 mM the parent compound #14a (4-hydroxyisoleucine) caused a limited stimulation of glucose uptake beyond that caused by insulin alone. However, at the same concentration the stimulation caused by analogs tested, i.e. mixture of compounds #128+#133, mixture of compounds #85(101a)+#101b and compound #13e was greater than the stimulation caused by the parent compound.

In summary, analogs 4-hydroxyisoleucine are capable of improved stimulation of glucose uptake in adipocytes relative to the parent compound 4-hydroxyisoleucine. This study thereby confirms the efficacy of the compound of the invention and provides hindsights for a structural design strategy of additional and/or more effective compounds.

### Example 3

### Glucose-Dependent Stimulation of Insulin Secretion in INS-1 Cells by Analogs of 4-hydroxyisoleucine

Selected analogs according to the invention were tested in a blinded fashion for insulinotropic effect on INS-1 cells. Briefly, the cells were plated at a density of 2×10⁵ in 12 well plates and incubated for 2 days in RPMI with 10% fetal calf serum and 11 mM glucose. The medium was removed on the third day post-plating and replaced with RPMI containing 3 mM glucose with 10% fetal calf serum. The cells were incubated for an additional 24 hours. On the fourth day post-plating, the medium was removed and replaced with Krebs-Ringer bicarbonate buffer containing 2 mM glucose. The cells were incubated for 30 min and the buffer was removed and replaced with Krebs-Ringer bicarbonate buffer with 4.5 mM glucose containing the compounds to be tested at a concentration of 0.5 mM. The cells were incubated for 1 hour. Basal insulin secretion was determined by incubating the cells in the presence of buffer with 2 mM glucose. The presence of glucose at 4.5 and 10 mM stimulated insulin secretion served as the reference control and positive control, respectively.

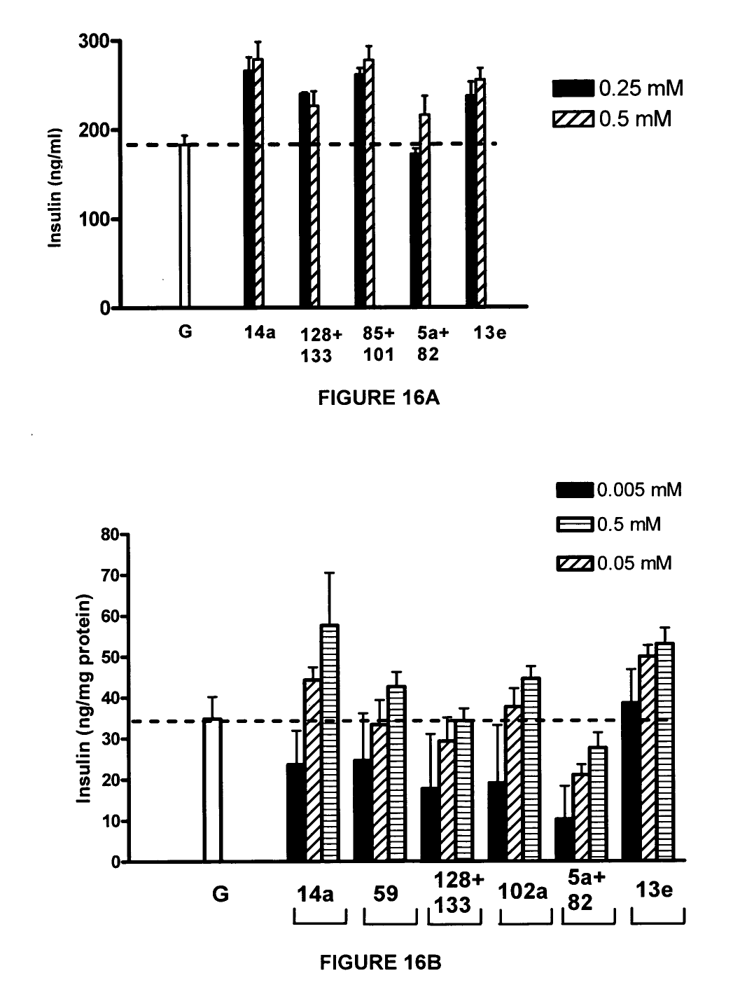

FIG. 16A shows the insulin stimulating activity in presence of with 4.5 mM glucose (G). As expected, parent compound #14a showed a significant insulin stimulating activity. All the analogs tested showed a stimulatory effect with a mixture of compounds #85(101a)+#101b, and compound #13e being the most effective.

FIG. 16B is another figure showing insulin stimulating activity of selected analogs in presence of 4.5 mM glucose (G). Most of the analogs tested showed a stimulatory effect, compound #13e being the most effective.

In summary, analogs 4-hydroxyisoleucine can stimulate insulin secretion, some at levels at least equivalent to the parent compound #14a (4-hydroxyisoleucine). This study thereby confirms the efficacy of the compound of the invention and provides hindsights for a structural design strategy of additional and/or more effective compounds.

### Example 4

### Glucose-Dependent Stimulation of Insulin Secretion in INS-1 Cells by Additional Analogs of 4-hydroxyisoleucine

Selected analogs according to the invention were screened for insulinotropic effect on INS-1 pancreatic beta cells according to the method described in Example 3.

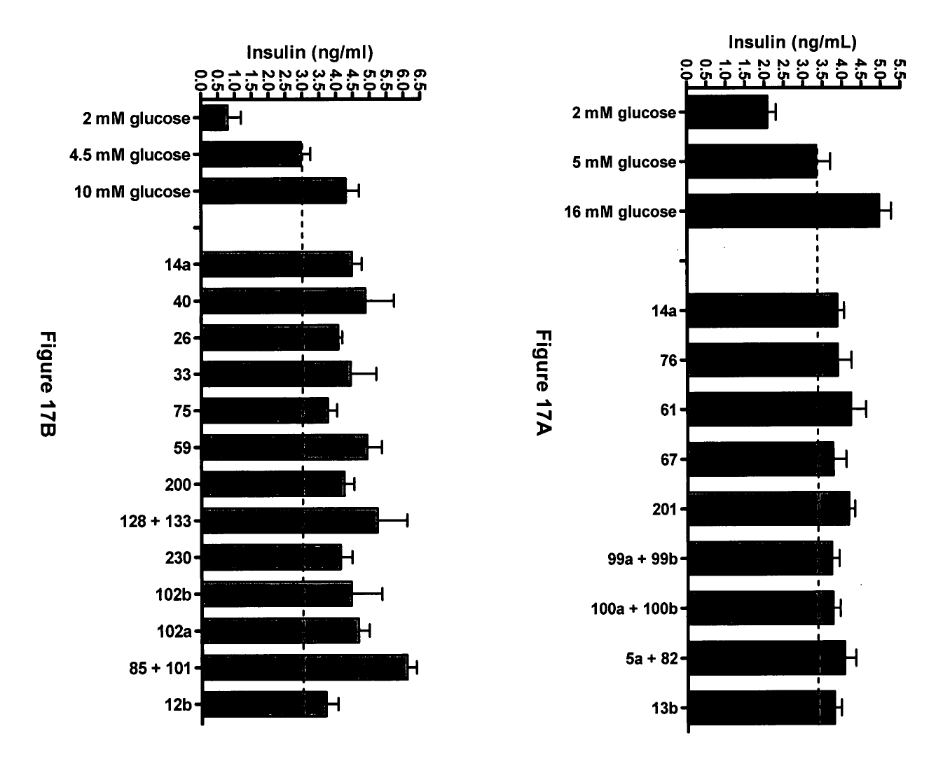

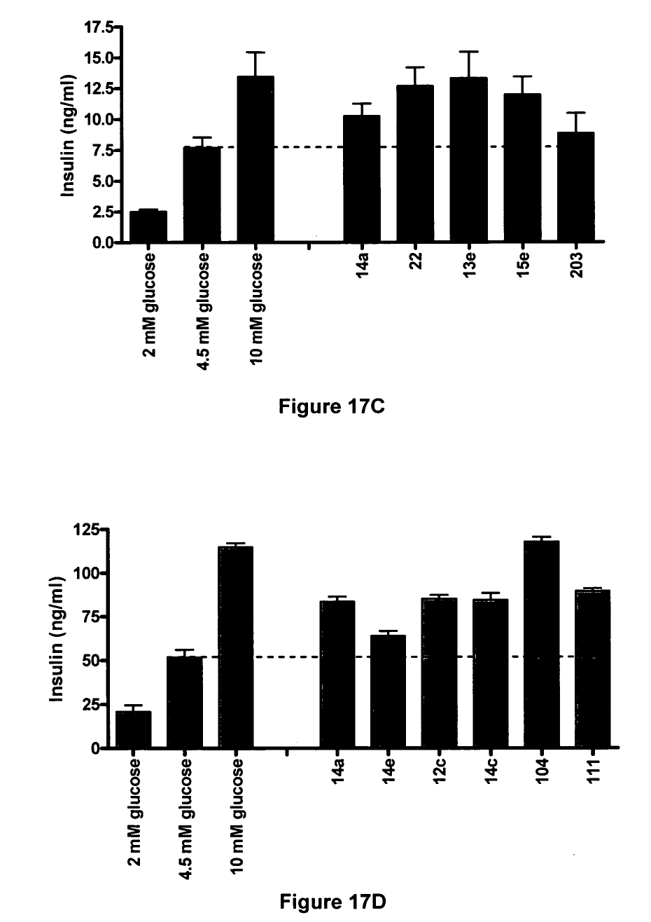

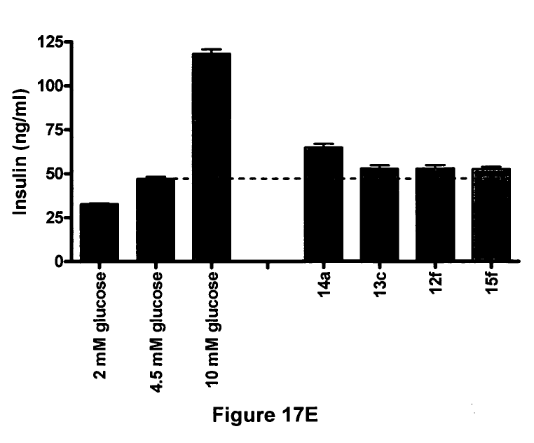

FIGS. 17A, 17B, 17C, 17D, and 17E show the stimulation of insulin secretion induced by the selected analogs (at a single concentration of 0.5 mM) in presence of 4.5 or 5 mM glucose. As expected, parent compound #14a (4-hydroxyisoleucine) showed a significant insulin stimulating activity in all the experiments. All the analogs presented in these graphs showed a stimulatory effect compared to control. Some compounds, and isomers (e.g. Compound #61, Compound #201, mixture #5a +82, #59, mixture #128+#133, mixture #85(101a)+#101b, Compound #22, Compound #13e, Compound #15e, Compound #104, and Compound #111) were all more effective to stimulate insulin secretion than parent compound #14a (4-hydroxyisoleucine).

Taking together these results demonstrate that several analogs of 4-hydroxyisoleucine can stimulate insulin secretion, some of them being even more effective than 4-hydroxyisoleucine (#14a).

The findings of Examples 2, 3 and 4 confirm the efficacy of the compounds of the invention and provide hindsight for a structural design strategy of additional and/or more effective compounds. These experiments further confirm that, similar 4-Hydroxyisoleucine, the analogs of the invention, and more particularly Compounds #13e and the mixture of isomers #85(101a)+#101b, have the potential to be used as therapeutic agents for preventing and treating disorders of carbohydrate or lipid metabolism, including diabetes mellitus (type 1 and type 2 diabetes), pre-diabetes and Metabolic Syndrome.

### Example 5

### Effect of synthetic analogs of (2S,3R,4S) 4-Hydroxyisoleucine on the Glycemic Response of Diet Induced Obesity (DIO)-C57BL/6 Mice Following a Single Oral Administration

Studies were conducted to evaluate the effect of acute oral administration of selected analogs according to the invention on the glycemic response of Diet-Induced Obesity (DIO)-C57BI/6 mice following an Oral Glucose Tolerance Test (OGTT).

In the first study, a total of 40 mice were used. The animals were randomized according to their basal glycemia values following a 5±0.5 hours fasting period and then distributed into 5 groups (1 control and 4 treated groups). Each group was composed of 8 animals. On the day of treatment, test articles were dissolved in reverse osmosis water and kept on ice. Control group (Group 1) received sterile water and Group 2 to 5 received 100 mg/kg of Compound #14a, Compound #128, Compound #133 and Compound #13e, respectively.

On the day of experimentation, animals were fasted ˜5 hours prior to the OGTT and then OGTT was performed at 10 minutes post-test article administration, by oral gavage administration of 40% glucose solution. Whole blood glucose levels were monitored using a hand-held glucometer prior to OGTT and for up to 2 hours post-lucose challenge.

In a separate experiment, the effect of acute oral administration of another analog, Compound #85(101a), on the glycemic response of DIO-mice was evaluated using the same experimental design.

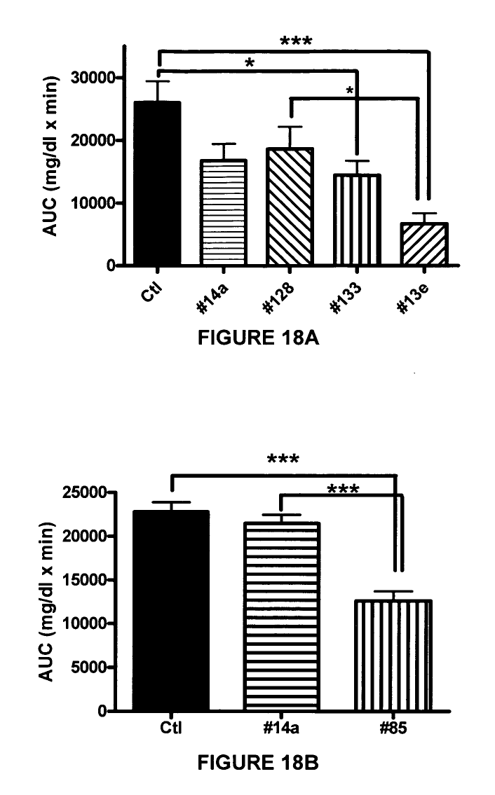

FIGS. 18A and 18B show the glycemic response of mice following an OGTT performed after a single oral administration of Compounds #14a, #128, #133, #13e, and #85(101a). For both figures, delta glycemia values were calculated by substraction of pre-OGTT glycemia value. AUC values were obtained from the delta glycemia curves. Values represent the mean±SEM. N=8 animals/group. Ctl=Control DIO.*p<0.05; . . . p<0.001.

No major clinical sign or mortality related to test articles was observed following the administration of the compounds. Following administration of glucose, all test agents lowered glycemia compared to control group (FIGS. 18A and 18B). Compounds #14a, #133, #85(101a) and #13e showed a significant effect on the glycemic control of DIO-mice. Compound #85(101a) and Compound #13e were the most efficacious compounds among those tested.

### Example 6

### Effect of Synthetic Analogues of (2S,3R.4S) 4-Hydroxyisoleucine on the Glycemic Response of Diet Induced Obesity (DIO)-C57BL/6 Mice following a Chronic Oral Administration

Studies were conducted to evaluate the effect of a chronic oral administration of selected analogs according to the invention on the glycemic response of Diet-induced Obesity (DIO)-C57BI/6 mice following an Oral Glucose Tolerance Test (OGTT) performed weekly.

In a first study, a total of 56 animals were used. The animals were distributed into 7 groups (6 treated and 1 high fat diet control groups). Each group was composed of 8 animals. The mice were randomized according to basal glycemia values following a 5±0.5 hours fasting period. Compound #14a and #133 were dissolved in reverse osmosis water while Compound #13e was dissolved in 200 mM Bicarbonate/0.1% Tween-20 buffer, pH=9.0. Compounds #14a and #133 were kept at 4° C (administration to groups 2 & 3, and 4 & 5, respectively) while Compound #13e was freshly prepared daily. Control animals received sterile saline, twice daily (Groups 1 and 2). Mice from groups 2 and 3 were treated twice daily with Compound #14a at 50 and 100 mg/kg, respectively. Animals from groups 4 and 5 received twice daily 50 and 100 mg/kg of Compound #133, respectively. Mice from groups 6 and 7 received 25 and 50 mg/kg of Compound #13e, twice daily, respectively.

On day 0, 7, 14, and 21, animals fasted for about 5 hours were challenged with an Oral Glucose Tolerance Test (OGTT) at 5 hours post-AM test article administration. Whole blood glucose levels were monitored using a hand-held glucometer prior to OGTT and for up to 2 hours post-glucose challenge. In a separate experiment, the same experimental design was used to evaluate the effect of chronic administration of Compound #85(101a) on the glycemic response of DIO-C57BI/6 mice following an Oral Glucose Tolerance Test (OGTT) performed after 7 days of treatment.

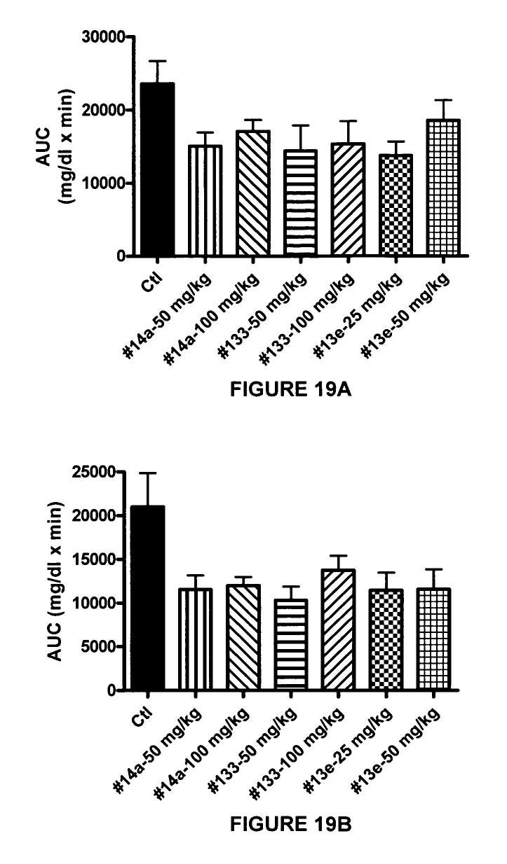

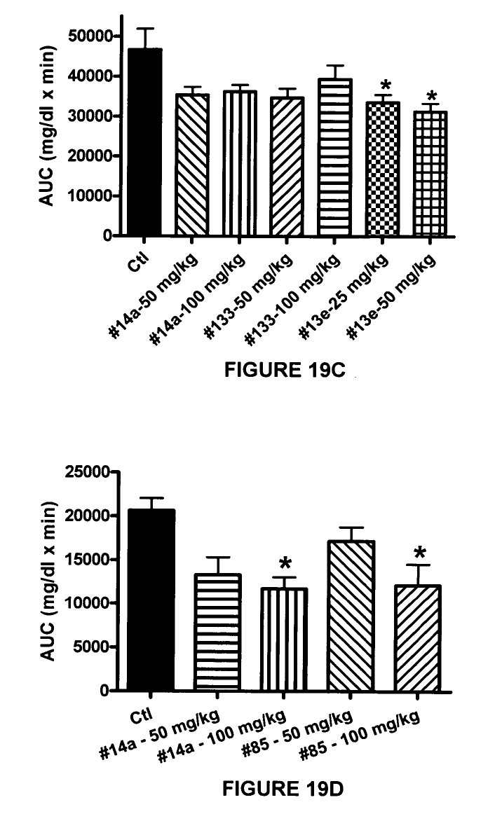

FIGS. 19A, 19B, 19C, and 19D are bar graphs showing glycemic response of mice following an OGTT performed after 7 days (FIGS. 19A and 19D), 14 days (FIG. 19B) or 21 days (FIG. 19C) of treatment after chronic oral administration of selected analogs according to the invention. For each figure, delta glycemia values were calculated by substraction of pre-OGTT glycemia value. AUC values were obtained from the delta glycemia curves. Values represent the mean±SEM. N=8 animals/group. Ctl=Control DIO.*p<0.05.

All compounds showed a beneficial effect on the control of glycemia, the maximal effect being observed at 14 days post-initiation of treatment for Compounds #14a, #133, and #13e. Compound #13e, given at half the dose (25 and 50 mg/kg) compared to the other treated groups (50 and 100 mg/kg), significantly reduced the glycemia increase in DIO-mice, suggesting that this compound could be more potent than other compounds. Moreover, Compound #13e at 25 and 50 mg/kg reduced significantly the hyperglycaemic response of animals at Day 21. These results suggest that Compound #13e could possess superior therapeutic activities since equal or superior efficacy was obtained with lower doses of the compound. Administration of Compound #85(101a) for 7 consecutive days also improved the glycemic control of DIO-mice and this effect was statistically significant when compared to the control group (FIG. 19D).

It is understood that the examples and embodiments described herein are for illustrative purposes only and that various modifications or changes in light thereof will be suggested to persons skilled in the art and are to be included within the spirit and purview of this application and scope of the appended claims.

What is claimed is:

1. A compound having the formula: 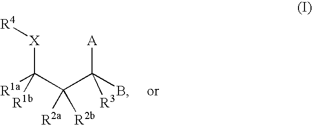
a pharmaceutically acceptable lactone, salt or prodrug thereof, wherein
A is CO₂R^{A1}, C(O)SR^{A1}, C(S)SR^{A1}, C(O)NR^{A2}R^{A3}, C(S)NR^{A2}R^{A3}, C(O)R^{A4}, SO₃H, S(O)₂NR^{A2}R^{A3}, C(O)R^{A5}, C(OR^{A1})R^{A9}R^{A10}, C(SR^{A1})R^{A9}R^{A10}, C(═NR^{A1})R^{A5}, 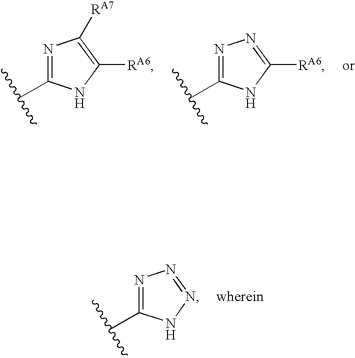
R^{A1} is hydrogen, substituted or unsubstituted C₁₋₆ alkyl, substituted or unsubstituted C₃₋₈ cycloalkyl, substituted or unsubstituted alkcycloalkyl, where the cycloalkyl group is of three to eight carbon atoms and the alkylene group is of one to four carbon atoms, substituted or unsubstituted C₂₋₆ alkenyl, substituted or unsubstituted C₂₋₆ alkynyl, substituted or unsubstituted C₆ or C₁₀ aryl, substituted or unsubstituted C₇₋₁₆ alkaryl, where the alkylene group is of one to four carbon atoms, substituted or unsubstituted C₁₋₉ heterocyclyl, or substituted or unsubstituted C₂₋₁₅ alkheterocyclyl, where the alkylene group is of one to four carbon atoms,
each of R^{A2} and R^{A3} is, independently, selected from the group consisting of (a) hydrogen, (b) substituted or unsubstituted C₁₋₆ alkyl, (c) substituted or unsubstituted C₃₋₈ cycloalkyl, (d) substituted or unsubstituted alkcycloalkyl, where the cycloalkyl group is of three to eight carbon atoms and the alkylene group is of one to four carbon atoms, (e) substituted or unsubstituted C₆ or C₁₀ aryl, and (f) substituted or unsubstituted C₇₋₁₆ alkaryl, where the alkylene group is of one to six carbon atoms, or R^{A2} taken together with R^{A3} and N forms a substituted or unsubsituted 5- or 6-membered ring, optionally containing O or NR^{A8}, wherein
R^{A8} is hydrogen or C₁₋₆ alkyl,
R^{A4} is substituted or unsubstituted C₁₋₆ alkyl, substituted or unsubstituted C₃₋₈ cycloalkyl, substituted or unsubstituted alkcycloalkyl, where the cycloalkyl group is of three to eight carbon atoms and the alkylene group is of one to four carbon atoms, substituted or unsubstituted C₆ or C₁₀ aryl, substituted or unsubstituted C₇₋₁₆ alkaryl, where the alkylene group is of one to four carbon atoms, substituted or unsubstituted C₁₋₉ heterocyclyl, or substituted or unsubstituted C₂₋₁₅ alkheterocyclyl, where the alkylene group is of one to four carbon atoms,
R^{A5} is a peptide chain of 1-4 natural or unnatural amino acids, where the peptide is linked via its terminal amine group to C(O),
each of R^{A1} and R^{A7} is, independently, hydrogen, substituted or unsubstituted C₁₋₆ alkyl, C₁₋₄ perfluoroalkyl, substituted or unsubstituted C₁₋₆ alkoxy, amino, C₁₋₆ alkylamino, C₂₋₁₂ dialkylamino, N-protected amino, halo, or nitro, and
each of R^{A9} and R^{A10} is, independently, selected from the group consisting of (a) hydrogen, (b) substituted or unsubstituted C₁₋₆ alkyl, (c) substituted or unsubstituted C₃₋₈ cycloalkyl, (d) substituted or unsubstituted alkcycloalkyl, where the cycloalkyl group is of three to eight carbon atoms and the alkylene group is of one to four carbon atoms, (e) substituted or unsubstituted C₆ or C₁₀ aryl, and (f) substituted or unsubstituted C₇₋₁₆ alkaryl, where the alkylene group is of one to six carbon atoms, or R^{A9} taken together with R^{A10} and their parent carbon atom forms a substituted or unsubsituted 5- or 6-membered ring, optionally containing O or NR^{A8}, wherein R^{A8} is hydrogen or C₁₋₆ alkyl;
B is NR^{B1}R^{B2}, wherein
(i) each of R^{B1} and R^{B2} is, independently selected from the group consisting of (a) hydrogen, (b) an N-protecting group, (c) substituted or unsubstituted C₁₋₆ alkyl, (d) substituted or unsubstituted C₂₋₆ alkenyl, (e) substituted or unsubstituted C₂₋₆ alkynyl, (f) substituted or unsubstituted C₃₋₈ cycloalkyl, (g) substituted or unsubstituted alkcycloalkyl, where the cycloalkyl group is of three to eight carbon atoms, and the alkylene group is of one to ten carbon atoms, (h) substituted or unsubstituted C₆ or C₁₀ aryl, (i) substituted or unsubstituted C₇₋₁₆ alkaryl, where the alkylene group is of one to six carbon atoms, (j) substituted or unsubstituted C₁₋₉ heterocyclyl, (k) substituted or unsubstituted C₂₋₁₅ alkheterocyclyl, where the alkylene group is of one to six carbon atoms, (I) C(O)R^{B3}, where R^{B3} is selected from the group consisting of substituted or unsubstituted C₁₋₆ alkyl, substituted or unsubstituted C₆ or C₁₀ aryl, substituted or unsubstituted C₇₋₁₆ alkaryl, where the alkylene group is of one to six carbon atoms, substituted or unsubstituted C₁₋₉ heterocyclyl, or substituted or unsubstituted C₂₋₁₅ alkheterocyclyl, where the alkylene group is of one to six carbon atoms, (m) CO₂R^{B4}, where R^{B4} is selected from the group consisting of substituted or unsubstituted C₁₋₆ alkyl, substituted or unsubstituted C₆ or C₁₀ aryl, substituted or unsubstituted C₇₋₁₆ alkaryl, where the alkylene group is of one to six carbon atoms, substituted or unsubstituted C₁₋₉ heterocyclyl, or substituted or unsubstituted C₂₋₁₅ alkheterocyclyl, where the alkylene group is of one to six carbon atoms, (n) C(O)NR^{B5}R^{B6}, where each of R^{B5} and R^{B6} is, independently, selected from the group consisting of hydrogen, substituted or unsubstituted C₁₋₆ alkyl, substituted or unsubstituted C₆ or C₁₀ aryl, substituted or unsubstituted C₇₋₁₆ alkaryl, where the alkylene group is of one to six carbon atoms, substituted or unsubstituted C₁₋₉ heterocyclyl, and substituted or unsubstituted C₂₋₁₅ alkheterocyclyl, where the alkylene group is of one to six carbon atoms, or R^{B5} taken together with R^{B6} and N forms a substituted or unsubsituted 5- or 6-membered ring, optionally containing a non-vicinal O, S, or NR′, where R′ is H or C₁₋₆ alkyl, (o) S(O)₂R^{B7}, where R^{B7} is selected from the group consisting of substituted or unsubstituted C₁₋₆ alkyl, substituted or unsubstituted C₆ or C₁₀ aryl, substituted or unsubstituted C₇₋₁₆ alkaryl, where the alkylene group is of one to six carbon atoms, substituted or unsubstituted C₁₋₉ heterocyclyl, or substituted or unsubstituted C₂₋₁₅ alkheterocyclyl, where the alkylene group is of one to six carbon atoms, and (p) a peptide chain of 1-4 natural or unnatural alpha-amino acid residues, where the peptide is linked via its terminal carboxy group to N, with the proviso that no two groups are bound to the nitrogen atom through a carbonyl group or a sulfonyl group, or
(ii) R^{B1} taken together with R^{B2} and N forms a substituted or unsubstituted 5- or 6-membered ring, optionally containing O or NR^{B8}, wherein R^{B8} is hydrogen or C₁₋₆ alkyl, or
(iii) a 5- to 8-membered ring is formed when R^{B1} taken together with R^{1a} is a substituted or unsubstituted C₁₋₄ alkylene, or
(iv) a [2.2.1] or [2.2.2] bicyclic ring system is formed when R^{B1} taken together with R^{1a} is a substituted or unsubstituted C₂ alkylene and R^{B1} taken together with R^{2a} is a substituted or unsubstituted C₁₋₂ alkylene, or
(v) a 4- to 8-membered ring is formed when R^{B1} taken together with R³ is a substituted or unsubstituted C₂₋₆ alkylene, or
(vi) a 6- to 8-membered ring is formed when R^{B1} taken together with R⁴ is a substituted or unsubstituted C₁₋₃ alkylene, or
(vii) R^{B1} taken together with A and the parent carbon of A and B forms the following ring: 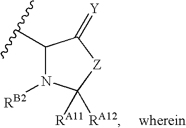
each of Y and Z is, independently, O, S, NR^{B8}, or CR^{A9}R^{A10}, wherein each of R^{A9} and R^{A10} is as previously defined and each of R^{A11} and R^{A12} is, independently, selected from the group consisting of (a) hydrogen, (b) substituted or unsubstituted C₁₋₆ alkyl, (c) substituted or unsubstituted C₃₋₈ cycloalkyl, (d) substituted or unsubstituted alkcycloalkyl, where the cycloalkyl group is of three to eight carbon atoms and the alkylene group is of one to four carbon atoms, (e) substituted or unsubstituted C₆ or C₁₀ aryl, and (f) substituted or unsubstituted C₇₋₁₆ alkaryl, where the alkylene group is of one to six carbon atoms, or R^{A9} taken together with R^{A10} and their parent carbon atom forms a substituted or unsubsituted 5-or 6-membered ring, optionally containing O or NR^{A8}, wherein R^{A8} is hydrogen or C₁₋₆ alkyl;
X is O, S, or NR^{X1}, where R^{X1} is selected from the group consisting of (a) hydrogen, (b) an N-protecting group, (c) substituted or unsubstituted C₁₋₆ alkyl, (d) substituted or unsubstituted C₂₋₆ alkenyl, (e) substituted or unsubstituted C₂₋₆ alkynyl, (f substituted or unsubstituted C₃₋₈ cycloalkyl, (g) substituted or unsubstituted alkcycloalkyl, where the cycloalkyl group is of three to eight carbon atoms, and the alkylene group is of one to ten carbon atoms, (h) substituted or unsubstituted C₆ or C₁₀ aryl, (i) substituted or unsubstituted C₇₋₁₆ alkaryl, where the alkylene group is of one to six carbon atoms, (j) substituted or unsubstituted C₁₋₉ heterocyclyl, or (k) substituted or unsubstituted C₂₋₁₅ alkheterocyclyl, where the alkylene group is of one to six carbon atoms;
each of R^{1a} and R^{1b} is, independently, substituted or unsubstituted C₁₋₆ alkyl, substituted or unsubstituted C₃₋₈ cycloalkyl, substituted or unsubstituted alkcycloalkyl, where the cycloalkyl group is of three to eight carbon atoms and the alkylene group is of one to four carbon atoms, substituted or unsubstituted C₂₋₆ alkenyl, substituted or unsubstituted C₂₋₆ alkynyl, substituted or unsubstituted C₆ or C₁₀ aryl, substituted or unsubstituted C₇₋₁₆ alkaryl, where the alkylene group is of one to four carbon atoms, substituted or unsubstituted C₁₋₉ heterocyclyl, or substituted or unsubstituted C₂₋₁₅ alkheterocyclyl, where the alkylene group is of one to four carbon atoms, or R^{1a} together with R^{2a} and their base carbon atoms form a substituted or unsubstituted C₅₋₁₀ mono or fused ring system, or a 3- to 6-membered ring is formed when R^{1a} together with R⁴ is a substituted or unsubstituted C₁₋₄ alkylene;
each of R^{2a} and R^{2b} is, independently, hydrogen, substituted or unsubstituted C₁₋₆ alkyl, substituted or unsubstituted C₃₋₈ cycloalkyl, substituted or unsubstituted alkcycloalkyl, where the cycloalkyl group is of three to eight carbon atoms and the alkylene group is of one to four carbon atoms, substituted or unsubstituted C₂₋₆ alkenyl, substituted or unsubstituted C₂₋₆ alkynyl, substituted or unsubstituted C₆ or C₁₀ aryl, substituted or unsubstituted C₇₋₁₆ alkaryl, where the alkylene group is of one to four carbon atoms, substituted or unsubstituted C₁₋₉ heterocyclyl, or substituted or unsubstituted C₂₋₁₅ alkheterocyclyl, where the alkylene group is of one to four carbon atoms, or R^{2a} and R^{2b} together are ═O, ═N(C₁₋₆ alkyl), ═CR^{2c}R^{2d}, where each of R^{2c} and R^{2d} is, independently, hydrogen or substituted or unsubstituted C₁₋₆ alkyl, or a substituted or unsubstitued C₂₋₅ alkylene moiety forming a spiro ring, or R^{2a} together with R^{1a} and their base carbon atoms form a substituted or unsubstituted C₅₋₁₀ mono or fused ring system;
R³ is hydrogen, substituted or unsubstituted C₁₋₆ alkyl, substituted or unsubstituted alkcycloalkyl, where the cycloalkyl group is of three to eight carbon atoms and the alkylene group is of one to four carbon atoms, substituted or unsubstituted C₂₋₆ alkenyl, substituted or unsubstituted C₂₋₆ alkynyl, substituted or unsubstituted C₇₋₁₆ alkaryl, where the alkylene group is of one to four carbon atoms, or substituted or unsubstituted C₂₋₁₅ alkheterocyclyl, where the alkylene group is of one to four carbon atoms; and
R⁴ is hydrogen, substituted or unsubstituted C₁₋₆ alkyl, substituted or unsubstituted C₃₋₈ cycloalkyl, substituted or unsubstituted alkcycloalkyl, where the cycloalkyl group is of three to eight carbon atoms and the alkylene group is of one to four carbon atoms, substituted or unsubstituted C₂₋₆ alkenyl, substituted or unsubstituted C₂₋₆ alkynyl, substituted or unsubstituted C₆ or C₁₀ aryl, substituted or unsubstituted C₇₋₁₆ alkaryl, where the alkylene group is of one to four carbon atoms, substituted or unsubstituted C₁₋₉ heterocyclyl, or substituted or unsubstituted C₂₋₁₅ alkheterocyclyl, where the alkylene group is of one to four carbon atoms, or a 3- to 6-membered ring is formed when R⁴ together with R^{1a} is a substituted or unsubstituted C₁₋₄ alkylene, or a 6- to 8-membered ring is formed when R⁴ taken together with R^{B1} is a substituted or unsubstituted C₁₋₃ alkylene, with the proviso that said compound of Formula (I) is not a configurational isomer of 4-hydroxyisoleucine or a configurational isomer of 4-hydroxyisoleucine γ-lactone.

2. The compound claim 1, wherein said compound is a compound of Formula (II): 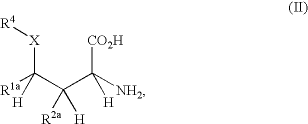
wherein each of X and R⁴ is as previously defined and each of R^{1a} and R^{2a} is, independently, substituted or unsubstituted C₁₋₆ alkyl or R^{1a} together with R^{2a} and their base carbon atoms form a substituted or unsubstituted 6 membered ring.

3. The compound claim 1, wherein said compound is a compound of Formula (III): 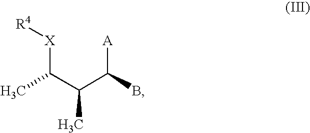
wherein A is CO₂R^{A1}, C(O)SR^{A1}, C(O)NR^{A2}R^{A3}, or C(O)R^{A5}, and wherein each of R^{A1}, R^{A2}, R^{A3}, R^{A5}, B, X, and R⁴ is as previously defined.

4. The compound of claim 1, wherein said compound is a compound of Formula (IV): 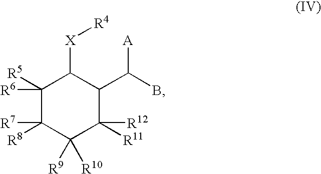
wherein A is CO₂R^{A1}, C(O)SR^{A1}, C(O)NR^{A2}R^{A3}, or C(O)R^{A5} and wherein each of B, X, and R⁴ is as previously defined, and wherein each of R⁵, R⁶, R⁷, R⁸, R⁹, R¹⁰, R¹¹, and R¹² is, independently, hydrogen, substituted or unsubstituted C₁₋₆ alkyl, substituted or unsubstituted C₃₋₈ cycloalkyl, substituted or unsubstituted alkcycloalkyl, where the cycloalkyl group is of three to eight carbon atoms and the alkylene group is of one to four carbon atoms, substituted or unsubstituted C₂₋₆ alkenyl, substituted or unsubstituted C₂₋₆ alkynyl, substituted or unsubstituted C₆ or C₁₀ aryl, substituted or unsubstituted C₇₋₁₆ alkaryl, where the alkylene group is of one to four carbon atoms, substituted or unsubstituted C₁₋₉ heterocyclyl, or substituted or unsubstituted C₂₋₁₅ alkheterocyclyl, where the alkylene group is of one to four carbon atoms.

5. The compound of claim 1, wherein said compound is: 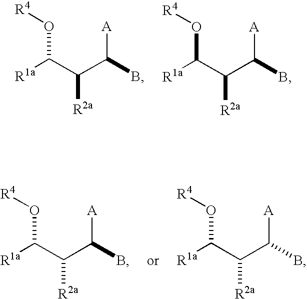
wherein each of R^{1a} and R^{2a} is, individually, substituted or unsubstituted C₁₋₆ alkyl, substituted or unsubstituted C₃₋₈ cycloalkyl, substituted or unsubstituted alkcycloalkyl, where the cycloalkyl group is of three to eight carbon atoms and the alkylene group is of one to four carbon atoms, substituted or unsubstituted C₂₋₆ alkenyl, substituted or unsubstituted C₂₋₆ alkynyl, substituted or unsubstituted C₆ or C₁₀ aryl, substituted or unsubstituted C₇₋₁₆ alkaryl, where the alkylene group is of one to four carbon atoms, substituted or unsubstituted C₁₋₉ heterocyclyl, or substituted or unsubstituted C₂₋₁₅ alkheterocyclyl, where the alkylene group is of one to four carbon atoms.

6. The compound of claim 1, wherein A is CO₂H, B is NH-p-toluenesulfonyl, R⁴ is H and each of R^{1a} and R^{2a} is CH₃.

7. The compound of claim 1, wherein A is CO₂H, B is NH₂, R⁴ is H and each of R^{1a} and R^{2a} is a substituted or unsubstituted C₁₋₆alkyl.

8. The compound of claim 1, wherein A is CO₂H, B is NH₂, X is O, and R⁴ is H.

9. The compound of claim 1, wherein said compound is 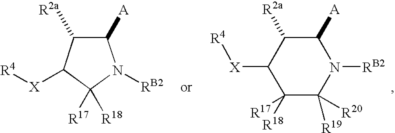
wherein each of A, X, R^{2a}, R⁴, and R^{B2} is as previously defined and wherein each of R¹⁷, R¹⁸, R¹⁹, and R²⁰ is hydrogen or substituted or unsubstituted C₁₋₆ alkyl.

10. The compound of claim 1, wherein said compound is 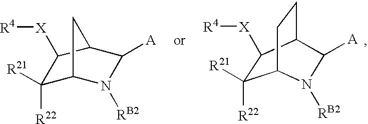
wherein each of A, X, R⁴, R²⁰, and R^{B2} is as previously defined, and wherein each of R²¹ and R²² is hydrogen or substituted or unsubstituted C₁₋₆ alkyl.

11. The compound of claim 1, wherein said compound is 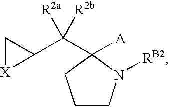
wherein each of A, X, R^{2a}, R^{2b} and R^{B2} is as previously defined.

12. The compound of claim 1, wherein said compound is 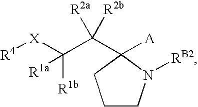
wherein each of A, X, R^{1a}, R^{1b} R^{2a}, R^{2b}, R⁴ and R^{B2} is as previously defined.

13. The compound of claim 1, wherein R^{1a} together with R^{2a} and their base carbon atoms form a substituted or unsubstituted C₅₋₁₀ mono or fused ring system, optionally containing a non-vicinal O, S, or NR′, where R′ is H or C₁₋₆ alkyl.

14. The compound of claim 1, wherein said compound of Formula (I) is selected from the group consisting of: 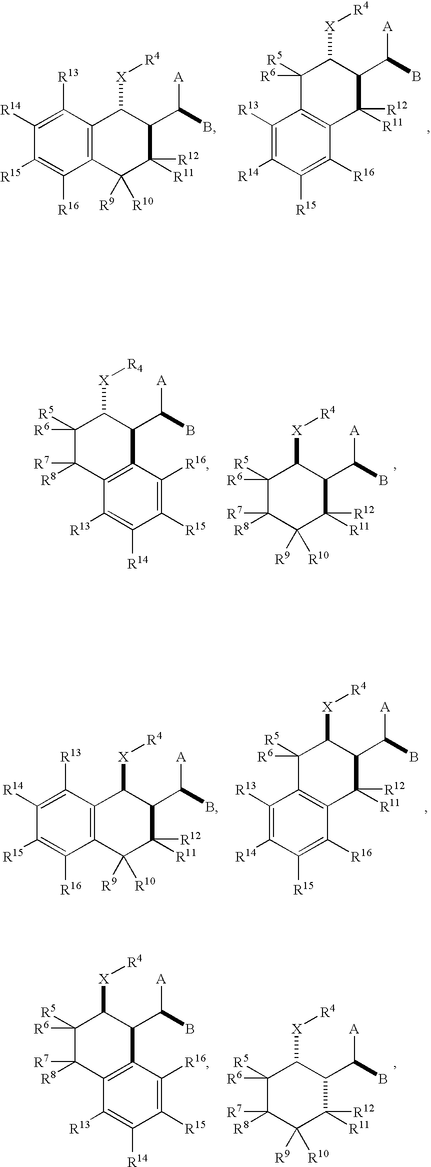 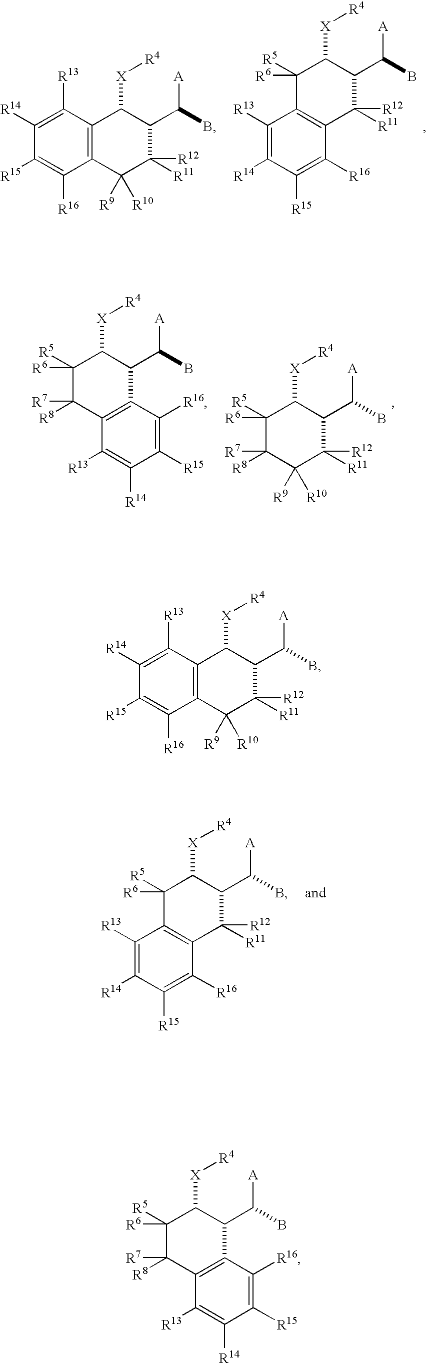
wherein each of R⁵, R⁶, R⁷, R⁸, R⁹ R¹⁰, R¹¹, and R¹² is, independently, hydrogen, substituted or unsubstituted C₁₋₆ alkyl, substituted or unsubstituted C₃₋₈ cycloalkyl, substituted or unsubstituted alkcycloalkyl, where the cycloalkyl group is of three to eight carbon atoms and the alkylene group is of one to four carbon atoms, substituted or unsubstituted C₂₋₆ alkenyl, substituted or unsubstituted C₂₋₆ alkynyl, substituted or unsubstituted C₆ or C₁₀ aryl, substituted or unsubstituted C₇₋₁₆ alkaryl, where the alkylene group is of one to four carbon atoms, substituted or unsubstituted C₁₋₉ heterocyclyl, or substituted or unsubstituted C₂₋₁₅ alkheterocyclyl, where the alkylene group is of one to four carbon atoms; and
each of R¹³, R¹⁴, R¹⁵, and R¹⁶ is, independently, hydrogen, substituted or unsubstituted C₁₋₆ alkyl, C₁₋₄ perfluoroalkyl, substituted or unsubstituted C₁₋₆ alkoxy, amino, C₁₋₆ alkylamino, C₂₋₁₂ dialkylamino, N-protected amino, halo, or nitro.

15. The compound of claim 1, wherein said compound is selected from the group consisting of:

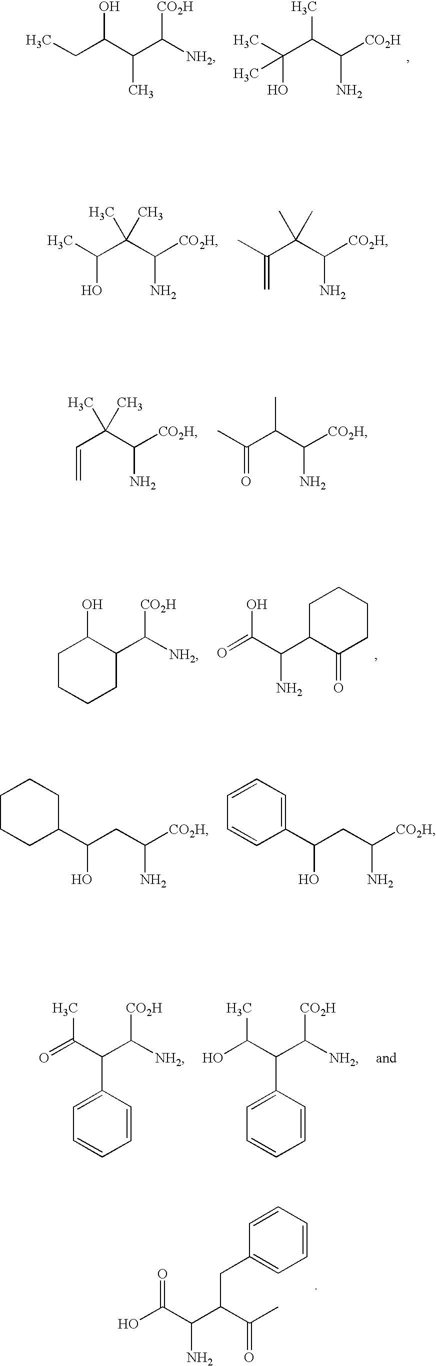

16. The compound of claim 1, wherein said compound is selected from the group consisting of:

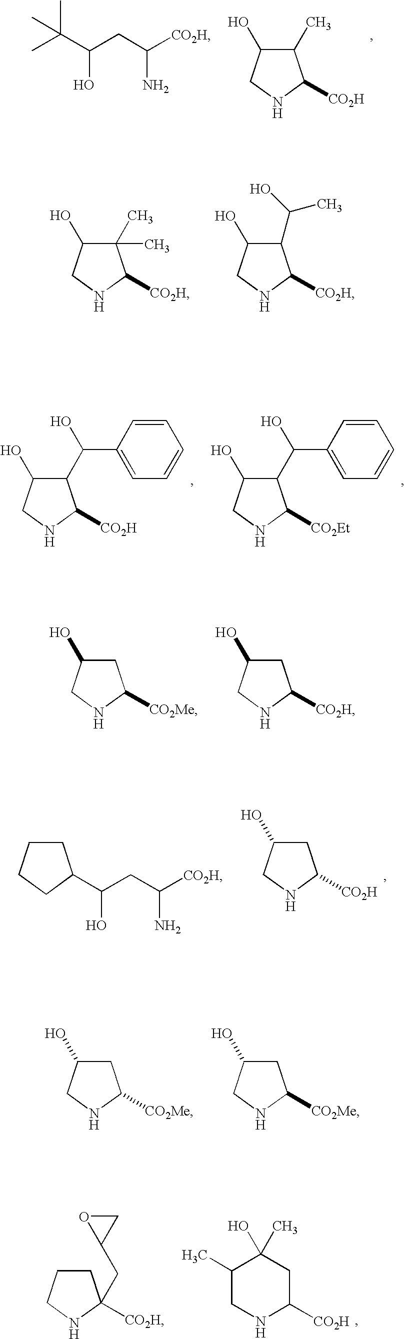

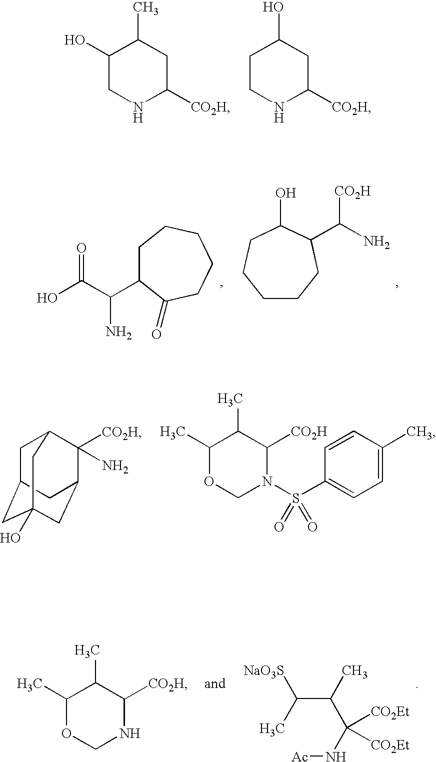

17. A compound having the formula: 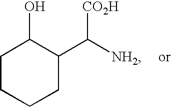
a pharmaceutically acceptable lactone, salt or prodrug thereof.

18. The compound of claim 17, or a pharmaceutically acceptable lactone, salt or prodrug thereof, wherein said compound has the following formula:

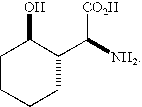

19. A pharmaceutical composition comprising a compound of claim 1, or a pharmaceutically acceptable salt, lactone or prodrug thereof, and a pharmaceutically acceptable carrier or excipient.

20. The pharmaceutical composition of claim 19, further comprising at least one antidiabetic agent selected from the list given in Table 2.

21. A pharmaceutical kit comprising a compound of claim 1, or a pharmaceutically acceptable salt, lactone or prodrug thereof, and instructions for the use of said compound for decreasing the circulating glucose level in a human patient.

22. A method for stimulating glucose uptake by muscle cells and/or adipocyte cells, comprising contacting said cells with an effective amount of a compound of claim 1.

23. A method for stimulating insulin secretion by pancreatic β-cells, comprising contacting said cells with an effective amount of a compound of claim 1.

24. A method for treating a mammal having a disorder of carbohydrate or lipid metabolism, said method comprising administering to said mammal a compound of claim 1.

25. The method of claim 24, wherein said disorder of carbohydrate metabolism is type 2 diabetes mellitus.

26. The method of claim 24, wherein said mammal is selected from the group consisting of primates, animals of agricultural and veterinary interest, rodents, and domestic pets.

27. The method of claim 24, wherein said mammal is a human.

28. A method for treating diabetes in a human, said method comprising administering to said human an antidiabetic compound in an amount sufficient to decrease the circulating glucose level in said human, wherein said antidiabetic compound is a compound of claims 1.

29. A method for the prevention or treatment of type 2 diabetes in a human, said method comprising administering to said human a compound having the formula: 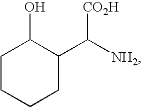
or a pharmaceutically acceptable lactone, salt or prodrug thereof.

30. The method of claim 29, wherein said compound has the following formula: [chemical structure]
